(19) United States
(12) Patent Application Publication
(10) Pub. No.: US 2013/0237486 A1
(43) Pub. Date: Sep. 12, 2013

(54) COLLAGEN

(71) Applicant: Jordi Bella, Manchester (GB)

(72) Inventor: Jordi Bella, Manchester (GB)

(73) Assignee: The University of Manchester, Manchester (GB)

(21) Appl. No.: 13/884,832

(22) PCT Filed: Nov. 14, 2011
(86) PCT No.: PCT/GB2011/052217

§ 371 Date: May 10, 2013

(30) Foreign Application Priority Data
Nov. 12, 2010 (GB) 1019143.5

(51) Int. Cl. C07K 14/78

(52) U.S. Cl. CPC C07K 14/78
USPC 514/17.2; 530/356; 536/23.4; 435/320.1; 435/419; 435/69.7; 435/348; 435/358; 435/365; 435/254.2; 435/252.3; 435/252.33; 435/252.31

(57) ABSTRACT

The present invention relates to a trimeric fusion protein comprising three polypeptide chains, wherein each polypeptide chain comprises a eukaryotic collagen or collagen-like domain and a prokaryotic or viral trimerisation domain (PVTD). Also provided is a fusion polypeptide comprising a eukaryotic collagen or collagen-like domain and a PVTD. A suitable PVTD of a fusion polypeptide or protein of the invention is preferably derived from a collagen-like protein sequence found in the genome of the E. coli strain O157:H7 and other E. coli strains, and in bacteriophages or prophages infecting these strains or embedded in their genomes. A PVTD mediates trimerisation of collagen or collagen like polypeptides.

The present invention relates to a trimeric fusion protein comprising three polypeptide chains, wherein each polypeptide chain comprises a eukaryotic collagen or collagen-like domain and a prokaryotic or viral trimerisation domain (PVTD). Also provided is a fusion polypeptide comprising a eukaryotic collagen or collagen-like domain and a PVTD. In addition, the present invention relates to a nucleic acid sequence encoding a fusion protein or polypeptide of the invention, an expression vector comprising a nucleic acid sequence of the invention, and a host cell comprising any one or more of a fusion protein, polypeptide, nucleic acid sequence or an expression vector of the invention. In addition, there are provided methods for the production of a fusion protein and/or polypeptide of the invention. Also provided is a product comprising any one or more of a fusion protein, polypeptide, nucleic acid sequence, expression vector or host cell of the invention, and uses any one or more of a fusion protein, polypeptide, nucleic acid sequence, expression vector or host cell in the manufacture of a product of the invention. Also provided are methods of treatment using any one or more of a fusion protein, polypeptide, nucleic acid sequence, expression vector, host cell or product of the invention.

### BACKGROUND

Collagens are structural proteins essential for building the macromolecular structures present in connective tissues such as bone, skin, cartilage, or blood vessel walls. Type 1 collagen, the most abundant form of collagen, is often used for treating skin injuries and is a commonly used bone restoration material. Many collagens contain cell-adhesion sites along their sequence. The interaction between these sites and cell-surface receptors has effects on cell proliferation and behaviour that can be exploited in tissue regeneration efforts. Collagen structures can also induce mineral deposition. There are mineral interaction sites on the surface of these structures, which can effectively induce and control the process of mineralization, promote bone formation, and induce bone formation in implants.

Collagens are the major structural macromolecules present in the extracellular matrix of metazoa, comprising approximately 20% of total protein mass. There are many different collagen types. In vertebrates, the count to date is fast approaching the thirties (Kadler et al., (2007) J. Cell Sci. 120:1955-1958) whereas worms can have hundreds of different collagen genes (Johnstone (2000) Trends Genet. 16: 21-27). Type I collagen, the main component of skin and bone, is the most abundant protein in humans and vertebrates comprising approximately 80-90% of an animals total collagen. Other collagen types are less abundant than type I collagen, and exhibit different distribution patterns. All collagens form trimeric associations; these trimers can form from three identical polypeptide chains coded by the same gene (homotrimers), or from different polypeptide chains coded by two or three different genes (heterotrimers). For example, type I collagen is a heterotrimeric molecule comprising two α1(I) chains and one α2(I) chain. Lack of agreed naming conventions mean that some collagen genes are labeled as belonging to different collagen types depending on the sources (for example the α5(VI) gene sequence is alternatively known as α1(XXIX), that is a different collagen type altogether). Different collagen types are expressed in different tissues.

Collagen types participate in some form of supramacromolecular assembly. The most abundant fibrillar collagens (types I, II, III) assemble into microfibrils, fibrils and fibres to provide the unique tensile properties of tendons, cartilage, skin, bone, and blood vessels. Type IV collagen forms networks that are responsible for the correct assembly of basement membranes, with important roles in molecular filtration (for example in kidney glomerulus).

Type VI collagen assembles to forms beaded-microfibrils, which provide structural links with cells in most tissues. Other less abundant collagen types can be associated to the structures built from the major types, where they act as regulatory elements, can appear as transmembrane molecules with cell-adhesive properties, can build anchoring fibrils, or can form networks in other membranous structures. A large and diverse group of “collagen-like” proteins contain collagen triple helical domains but are not universally classified as “collagens”. These include acetyl cholinesterase, macrophage scavenger receptor, surfactant pulmonary proteins, or C1q. The last three examples share a role in innate immune defence.

Collagen types I, II and III belong to a group of fibrillar collagens, characterised by the formation of 67-nm periodic fibrils that provide tensile strength to animal tissues. Type II collagen is a homotrimeric collagen comprising three identical α1(II) chains, and is the predominant collagen in cartilage and vitreous humour. Type III collagen is found in skin and vascular tissues and is also a homotrimeric collagen, comprising three identical α1(III) chains. Type IV collagen forms networks instead of fibrils and is found in basement membranes. There are several type IV collagen isoforms, the most common being a heterotrimer made of two α1(IV) chains and one α2(IV) chain. Type V collagen exists in both homotrimeric and heterotrimeric forms and is a minor fibrillar collagen found in tissues containing type I collagen. Type VI collagen has a small central triple helical region and two large non-collagenous domains. It is a heterotrimer comprising α1(VI), α2(VI), and α3(VI) chains and is found in many connective tissues forming beaded-filaments. Type VII collagen is a fibrillar collagen found in specialised epithelial tissues, and is a homotrimeric molecule of three α1(VII) chains. Type VIII collagen can be found in Descemet's membrane in the cornea and is a heterotrimer comprising two α1(VIII) chains and one α2(VIII) chain. Type IX collagen is a fibril-associated collagen found in cartilage and vitreous humor, and is a heterotrimeric molecule comprising α1(IX), α2(IX), and α3(IX) chains. Type IX collagen is the prototype of a group of collagens called FACIT (Fibril Associated Collagens with Interrupted Triple Helices), which contain several triple helical domains separated by non-triple helical domains.

Type X collagen is a homotrimeric compound of α1(X) chains and has been found in growth plates. Type XI collagen can be found in cartilaginous tissues associated with type II and type IX collagens, and in other locations in the body. Type XI collagen is a heterotrimeric molecule comprising α1(XI), α2(XI), and α3(XI) chains. Type XII collagen is a FACIT collagen found primarily in association with type I collagen. Type XII collagen is a homotrimeric molecule comprising three α1(XII) chains. Type XIII collagen is a homotrimeric non-fibrillar collagen found, for example, in skin, intestine, bone, cartilage, and striated muscle. Type XIV is a FACIT collagen characterized as a homotrimeric molecule comprising α1(XIV) chains. Type XV collagen is homologous in structure to type XVIII collagen. Type XVI collagen is a fibril-associated collagen found, for example, in skin, lung fibroblast, and keratinocytes. Type XVII collagen is a hemidesmosal transmembrane collagen, also known as the bullous pemphigoid antigen. Type XVIII collagen is similar in structure to type XV collagen and can be isolated from the liver. Type XIX collagen is believed to be another member of the FACIT collagen family, and has been found in mRNA isolated from rhabdomyosarcoma cells. Type XX collagen is a newly found member of the FACIT collagenous family, and has been identified in chick cornea.

The three dimensional structure of collagen has taken many years to elucidate, and its study has been facilitated by the use of synthetic collagen-related peptides (Brodsky & Persikov (2005) Adv. Protein Chem. 70:301-339; Okuyama (2008) Connect. Tissue Res. 49:299-310) for example in crystallographic analyses (Okuyama et al (1981) J. Mol. Biol. 152:427-443; Bella et al. (1994), Science 266:75-81; Kramer et al. (1999), Nat. Struct. Biol. 6:454-457; Kramer et al J. Mol. Biol. 301: 1191-1205; Bella et al. (2006), J. Mol. Biol. 362:298-311; Bella (2010), J. Struct. Biol., 170: 377-391). The use of synthetic collagen model peptides containing specific recognition motifs has allowed the investigation of receptor-binding properties of different collagen types (Farndale et al. (2008), Biochem. Soc. Trans. 36:241-250).

Collagen proteins are now known to include a triple helical domain where three polypeptide strands are wound around each other. The three polypeptide strands, known as alpha chains, each adopt a left-handed helical conformation.

This triple helical arrangement is the main structural feature of all collagen proteins and is known as the collagen triple helix (Brodsky supra). The defining characteristic of this structure is the supercoiling of the three polypeptide strands, each of which adopts a polyproline II left-handed helical conformation. These three left-handed helices are twisted together with one residue vertical staggering to form a right-handed superhelix. A continuous ladder of intermolecular backbone hydrogen bonds stabilise the triple helical structure. Collagen triple helices can span very long lengths: the collagen triple helix of type I collagen is typically over 300 nm in length and in excess of 1000 amino acids.

The main form of human collagen in the body (type I collagen) is formed from three polypeptide chains, which are first synthesized as preprocollagen. Each preprocollagen chain contains, in addition to the sequence of the mature collagen protein, one N-terminal propeptide and one C-terminal propeptide (known as registration peptides), and a signal peptide. During post-translational modification of the preprocollagen, the signal peptide is cleaved off in the endoplasmic reticulum, to provide procollagen chains. Within the rough endoplasmic reticulum, the procollagen chains combine to form a procollagen triple helix, still carrying the propeptides (registration peptides). The procollagen triple helix is then transported to the Golgi apparatus, where it is prepared for export from the cell. Once outside the cell, registration peptides are cleaved and procollagen peptidase converts the procollagen triple helix to the mature form, tropocollagen, containing a collagen triple helical domain and two remaining telopeptides flanking each side of the triple helical domain (see Kadler et al. (1996), Biochem. J. 316:1-11, for a review of fibrillar collagen synthesis and fibril formation). Tropocollagen molecules then aggregate to form fibrils, which in turn form collagen fibres. The collagen may be attached to the cell surface by binding molecules such as integrin and fibronectin. Other collagen types have similarly complex biosynthesis pathways.

In type I collagen, and possibly in all fibrillar collagens, triple helices conform into higher order structures known as microfibrils. Each microfibril associates with neighbouring microfibrils to produce a stable, crystalline, structure (Orgel et al. (2006) Proc. Natl. Acad. Sci. USA 103:9001-9005). The fibrils resulting from the assembly of such collagen triple helices exceed 1 μm in length.

A distinct feature of triple helical domains is the characteristic Gly-X-Y repeating sequence in each of the three polypeptide chains of the triple helix. The X position is often occupied by proline residues (Pro) and the Y position is often occupied by 4-hydroxyproline residues (Hyp), which are the result of post-transcriptional modification of prolines in the Y position of Gly-X-Y repeating sequences (Myllyharju (2003), Matrix Biol. 22:15-24). Thus, proline or hydroxylproline make up about a sixth of the amino acid residues in the most abundant collagen types. Due to its role in determination of cell type, cell adhesion, tissue regulation and infrastructure, collagen is not a simple structural protein which would typically lack chemically reactive side chains. In fact, many of the non-proline rich regions of collagen are cell or matrix associated and have regulatory roles. This has the result that mutations which affect the formation of collagen can have serious pathological effects, in humans, at least.

Collagen was initially thought to be exclusive to vertebrates, but has also been found in lower invertebrates such as sponges, mussels, and worms. More recently, sequencing of bacterial and viral genomes has revealed an unexpected number of sequences containing the landmark Gly-X-Y sequence (Rasmussen et al. (2003) J. Biol. Chem. 278:32313-32316). In a few cases it has been demonstrated that the bacterial regions with Gly-X-Y sequences adopt the triple helical conformation and correspond to triple helical domains (Xu et al. (2002) J. Biol. Chem. 277:27312-27318).

US Patent Application No. US2004/0214282 provides recombinant triple helical proteins comprising bacterial and mammalian collagen. Methods for the production of recombinant prokaryotic collagen-like proteins based on collagen-like sequences from Streptococcus pyogenes are provided by U.S. Pat. No. 7,544,780 and US Patent Application No. US2009/0258390.

Collagen is widely used in the cosmetic and pharmacological industries, for example as a stabiliser, in pill coatings and capsules, and in dietary supplements. In addition, denatured collagen (known as gelatine) is widely used in foodstuffs, such as desserts. Collagen for industrial uses is typically obtained from animal sources, mainly bovine and swine or more recently from cadavers, placentas or foetuses. However, these animal-derived collagen products can often be contaminated by viruses and prions, and can induce autoimmune diseases when tested in animal models. In view of fears regarding prion related disease, in Europe and the US in particular, collagen must be free from potential prion and viral contamination.

Several strategies have been employed in order to induce triple-helical structure formation in isolated collagen sequences (U.S. Pat. No. 6,096,863). Triple-helix structure formation in isolated collagen sequences may be induced by adding a number of Gly-Pro-Hyp repeats to both ends of a collagenous sequence. However, even with more than 50% of the peptide sequence consisting of Gly-Pro-Hyp repeats, the resulting triple-helices may not have sufficient thermal stability to survive at physiological conditions. Although substantial stabilization of the triple-helical structure may be achieved with the introduction of covalent links between the C-terminal regions of the three peptide chains, the large size (90-125 amino acid residues) of the resulting “branched” triple-helical peptide compounds make them difficult to synthesize and purify.

For these reasons, it would be advantageous to find an alternative to animal-derived collagen, which can be produced easily and in large quantities.

### BRIEF SUMMARY OF THE DISCLOSURE

Thus, in a first aspect of the present invention, there is provided a trimeric fusion protein comprising three polypeptide chains, wherein each polypeptide chain comprises a eukaryotic collagen or collagen-like domain and a prokaryotic or viral trimerisation domain (PVTD).

Preferably, fusion proteins of the invention have a trimeric structure, created by association of the three polypeptide chains. Preferably, the structure is a collagen or collagen-like structure, where the polypeptide chains are coiled together along their length. Optionally, a part of the fusion protein (for example one or more PVTDs) may comprise an alpha-helical coiled coil structure. Each polypeptide “chain” of the triple helix of the fusion protein may be comprised of two or more polypeptides.

Two or more of the three polypeptide chains may be the same as each other or may be different. Thus, the fusion protein may be a homotrimer or a heterotrimer. Preferably, the three polypeptide chains of the fusion protein are wound together, at least in part, to form a triple-helical structure. Preferably, trimerisation of the three polypeptide chains is mediated by one or more PVTDs.

Preferably, a fusion protein of the invention will have one or more of the following, independently selected, properties:
a) a melting temperature of between 34° C. and 60° C., preferably between 34° C. and 59° C., more preferably between 34° C. and 58° C., 57° C., 56° C., 55° C., 54° C., 53° C., 52° C., 51° C., 50° C., 49° C., 48° C., 47° C., 46° C., or 45° C., more preferably between 38° C. and 44° C., more preferably between 39° C. and 43° C., more preferably at least 40° C., 41° C. or 42° C.;
b) solubility of at least 25, at least 30, at least 31, at least 32, at least 33, at least 34, at least 35, at least 36, at least 37, at least 38, at least 39, or at least 40 mg/ml;
c) is comprised of one or more fusion polypeptides which are substantially resistant to proteolytic degradation by host enzymes when expressed in prokaryotic cells.

In addition, the fusion proteins of the invention may exhibit improved ability to refold (thermal reversibility) after denaturation into a collagen or collagen-like structure.

Herein, the melting temperature is defined as the temperature at which one or more of the PVTD's of the fusion protein denature (or dissociate) to form dimers or monomers. This is also known as a helix to coil transition. It may be the temperature at which any one of the PVTD's loses thermal stability and undergoes denaturation, or it may be the temperature at which all of the PVTD's in the fusion protein have substantially lost thermal stability (and undergone denaturation such that the trimeric structure is lost and replaced by separate monomers and/or dimers). Preferably, it is the latter, such that the fusion protein as a whole dissociates into separate monomers or dimers. Denaturation at the melting temperature may be complete or incomplete. Preferably it is the latter, so that the dimers or monomers (fusion polypeptides) become separate entities. Where more than one PVTD of different types are present in a fusion protein, these may have the same or different melting temperatures. The melting temperature of a PVTD of the fusion protein may be the same as, or may be different to, the melting temperature of the eukaryotic collagen of the fusion protein. Whilst the melting temperature of a eukaryotic collagen or collagen-like protein of the fusion protein may be higher than that of a PVTD, typically it will be lower, typically at least lower than that of the most thermally stable PVTD of the fusion protein. The melting temperature may be determined by any known method in the art. Suitable conditions under which the melting temperature may be determined, for example, are measuring the CD signal at 220 nm or 222 nm while varying the temperature. Alternatively, viscosity can be measured while varying the temperature. Preferably, fusion protein samples are provided in physiological conditions, for example approximately 10 nM Tris-HCL at pH 7.5, 150 mM NaCl. The temperature may be increased in any suitable increment, for example 20° C./hour.

The solubility of the fusion protein is defined as the extent to which the fusion protein dissolves in liquid, preferably water. The solubility is measured by any suitable means. For example, sample of fusion protein may be added dropwise to a liquid such as water until complete dissolution is observed. The concentration of fusion protein dissolved in the liquid indicates the solubility.

In a prokaryotic host cell typically, a fusion polypeptide will be degraded before it can assemble into a trimeric fusion protein. This is due to the absence in a prokaryotic host cell of an endoplasmic reticulum which protects unfolded proteins from degradation. Thus, it is difficult to obtain commercially useful yields of fusion protein in prokaryotic host cells. The fusion proteins of the present invention have the advantage that one or more of the PVTD's present reduce or prevent degradation of a fusion polypeptide by the host cell, thus allowing formation of a fusion protein within the host cell. By substantially preventing degradation is meant that at least 20%, 30%, 40%, 45%, 50%, 55%, 60%, 65%, 70%, 75%, 80%, 85%, 90% or at least 95% more fusion polypeptide is able to form a collagen or collagen-like fusion protein in a prokaryotic host cell than would be observed without one or more of the PVTD's present. The ability to avoid degradation by native host enzymes means that the fusion protein is capable of being expressed in the cell, and surviving in order to form a triple helical structure and preferably being harvested therefrom. Preferably, the fusion proteins of the invention comprise one or more PVTD which functions as a capping domain. Typical enzymes which degrade fusion polypeptides within a host cell include proteases, such as serine proteases, such as trypsin or chymotrypsin. Other enzymes will be known to persons skilled in the art.

In a second aspect of the invention, there is provided a fusion polypeptide comprising a eukaryotic collagen or collagen-like domain and a PVTD.

Preferably, the fusion protein and fusion polypeptide of the invention do not comprise prokaryotic or viral collagen domains. Thus, the collagen or collagen-like domain of a fusion protein or fusion polypeptide is preferably entirely eukaryotic.

In a third aspect of the invention, there is provided a nucleic acid sequence encoding a trimeric fusion protein comprising three polypeptide chains, wherein each polypeptide chain comprises a eukaryotic collagen or collagen-like domain and a PVTD. The fusion protein encoded by the nucleic acid is preferably as defined herein, preferably in accordance with the first aspect. Where the nucleic acid sequence encodes a fusion protein of the invention, the sequence encoding each polypeptide chain may be the same or different, such that the fusion protein is either a homotrimer or a heterotrimer. Also provided is a nucleic acid sequence encoding a fusion polypeptide comprising a eukaryotic collagen or collagen-like domain and a PVTD. Preferably, the fusion polypeptide is as disclosed herein preferably in accordance with the second aspect.

In a fourth aspect of the invention, there is provided a vector comprising a nucleic acid sequence encoding a trimeric fusion protein comprising three polypeptide chains, wherein each polypeptide chain comprises a eukaryotic collagen or collagen-like domain and a PVTD. The nucleic acid sequence is preferably as defined herein, preferably in accordance with the third aspect. Where the nucleic acid sequence encodes a fusion protein of the invention, the sequence encoding each polypeptide chain may be the same or different, such that the fusion protein is either a homotrimer or a heterotrimer. Also provided is an expression vector comprising a nucleic acid sequence encoding a fusion polypeptide comprising a eukaryotic collagen or collagen-like domain and a PVTD. Preferably, the nucleic acid sequence encoding the fusion protein or polypeptide is as described herein, preferably in accordance with the third aspect.

In a fifth aspect of the invention, there is provided a host cell comprising any one or more of a fusion protein, fusion polypeptide, nucleic acid sequence or vector of the invention, as described herein. The host cell may be of any cell type. It may be prokaryotic or eukaryotic. It may preferably be a bacteria, yeast, insect, mammalian or plant. Where bacterial, it is preferably gram negative, preferably E. coli, more preferably O157:H7.

In a sixth aspect of the invention, there is provided a method of producing a trimeric fusion protein comprising three polypeptide chains, wherein each polypeptide chain comprises a eukaryotic collagen or collagen-like domain and a PVTD, the method comprising:

i) introducing into a host cell one or more nucleic acid sequences encoding a fusion protein or fusion polypeptide of the invention;
ii) culturing the host cell under conditions suitable for expression of said fusion protein or fusion polypeptide and optionally formation of a trimeric fusion protein comprising three polypeptide chains;
iii) optionally isolating the expressed fusion protein or fusion polypeptide from the host cell.

Preferably, the fusion protein, fusion polypeptide, nucleic acid sequence and/or host cell used in the method is as herein.

Also provided is a method of producing a fusion polypeptide comprising a eukaryotic collagen or collagen-like domain and a PVTD, the method comprising:

i) introducing into a host cell a nucleic acid sequence encoding said fusion polypeptide of the invention;
ii) culturing the host cell under conditions suitable for expression of said fusion polypeptide;
iii) optionally isolating the expressed fusion polypeptide from the host cell.

Preferably, the fusion polypeptide, nucleic acid sequence, vector and host cell used in the method is as defined herein.

As an alternative method, the sixth aspect of the invention also provides a method of producing a fusion protein comprising three polypeptide chains, wherein each polypeptide chain comprises a eukaryotic collagen or collagen-like domain and a PVTD in a cell free system, the method comprising:

i) introducing into a cell-free expression system one or more nucleic acid sequences encoding said fusion protein or fusion polypeptide;
ii) maintaining the cell-free expression system under conditions suitable for expression of said fusion protein or fusion polypeptide and formation of a trimeric fusion protein comprising three of said polypeptide chains; and
iii) optionally isolating the expressed fusion protein or fusion polypeptide from the expression system.

Preferably, the fusion protein, fusion polypeptide, nucleic acid sequence, vector and/or host cell used in the method are as described herein.

Also provided is a method of producing a fusion polypeptide comprising a eukaryotic collagen or collagen-like domain and a PVTD, the method comprising:

i) introducing into a cell-free expression system a nucleic acid sequence encoding a fusion polypeptide of the invention;
ii) maintaining the cell-free expression system under conditions suitable for expression of said fusion polypeptide;
iii) optionally isolating the expressed fusion polypeptide from the host cell.

Preferably, the fusion polypeptide, nucleic acid sequence, vector and/or host cell are as described herein.

Preferably, the methods of the sixth aspect further comprise purifying the fusion protein or fusion polypeptide.

The present invention also provides any suitable method for making the fusion protein or fusion polypeptide of the invention, which may be available to a person skilled in the art. Such methods may include, for example, chemical synthesis of a fusion protein of the invention.

In a seventh aspect of the invention, there is provided a method of producing a gelatine-like protein, comprising:

i) introducing into a host cell one or more nucleic acid sequences encoding a fusion protein of the invention;
ii) culturing the host cell under conditions suitable for expression and formation of a trimeric fusion protein; and
iii) optionally isolating the expressed fusion protein from the host cell; and
iv) fully or partially denaturing and/or fragmenting a trimeric fusion protein of iii) to produce a gelatine-like protein.

Again, preferably the fusion protein, fusion polypeptide, nucleic acid sequence, vector and/or host cell are as described herein.

As an alternative method, the seventh aspect of the invention also provides a method of producing a gelatine-like protein, in a cell free system, the method comprising:

i) introducing into a cell-free expression system one or more nucleic acid sequences encoding a fusion protein of the invention;
ii) maintaining the cell-free expression system under conditions suitable for expression and formation of a trimeric fusion protein; and
iii) optionally isolating the expressed fusion protein from the expression system; and
iv) fully or partially denaturing and/or fragmenting a trimeric fusion protein of iii) to produce a gelatine-like protein. Alternatively, the method may comprise, after step iii), providing conditions for the formation of a trimeric fusion protein.

Again, preferably the fusion protein, fusion polypeptide, nucleic acid sequence, vector and/or host cell are as described herein.

In an alternative method, the seventh aspect of the invention provides a method of producing a gelatin-like protein, comprising:

i) introducing into a host cell one or more nucleic acid sequences encoding a fusion polypeptide;
ii) culturing the host cell under conditions suitable for expression of the fusion polypeptide; and
iii) optionally isolating the expressed fusion polypeptide from the host cell.

Preferably, the fusion protein, fusion polypeptide, nucleic acid sequence, vector and/or host cell are as defined herein.

Also provided is a method of producing a gelatin-like protein, in a cell-free system, the method comprising:

i) introducing into a cell-free expression system one or more nucleic acid sequences encoding said fusion polypeptide;
ii) maintaining a cell-free expression system under conditions suitable for expression of the fusion polypeptide; and
iii) optionally isolating the fusion polypeptide from the expression system to produce a gelatin-like protein.

Preferably, the fusion polypeptide, nucleic acid sequence are as defined herein, preferably that of the third aspect. The nucleic acid sequence may be provided in a host cell as an expression vector, preferably of the fourth aspect.

Preferably, the methods of the seventh aspect further comprise purifying the gelatine-like protein.

In an eighth aspect of the invention, there is provided a product comprising any one or more of a fusion protein, polypeptide, nucleic acid sequence, expression vector, gelatin-like protein or host cell of the invention. Such a product may be independently selected from the group consisting of a foodstuff, cosmetic, stabilizer, capsules, biomaterial, medical device, medicament, artificial tissue, pharmaceutical or nutritional supplement, chemical or biochemical reagent, or glue.

Also provided is a gelatin-like protein of the invention, which preferably comprises fusion polypeptides of the invention, partially or fully denatured fusion proteins of the invention, and/or fragments of fusion polypeptides or fusion proteins of the invention. Some of the fusions protein or fragments thereof may be trimeric or in a triple helical structure. Preferably, substantially all is denatured, or if trimeric, has substantially lost the triple helical formation.

Also provided is any one or more of a fusion protein, polypeptide, nucleic acid sequence, expression vector, gelatin-like protein, or host cell or product of the invention for use in the treatment or prevention of a collagen-related disorder.

Also provided is a method of treatment or prevention of a collagen-related disorder, comprising administrating to a subject any one or more of a fusion protein, nucleic acid sequence, expression vector, gelatine-like protein, host cell or product of the invention. The treatment may be cosmetic, to improve the appearance of a subject, or may be therapeutic.

In a final aspect of the invention, there is provided the use of any one or more of a fusion protein, nucleic acid sequence, expression vector gelatin-like protein, or host cell of the invention, in the manufacture of a product of the invention. As defined above, such a product may be independently selected from the group comprising of a foodstuff, cosmetic, stabilizer, capsules, biomaterial, medical device, medicament, artificial tissue, pharmaceutical or nutritional supplement, chemical or biochemical reagent, or glue.

### BRIEF DESCRIPTION OF THE DRAWINGS

The present invention is further described hereinafter with reference to the accompanying drawings and Tables, in which:

FIG. 1 shows domain architectures of several collagen-like proteins from prophages embedded in the genomes of E. coli O157:H7 and related strains, plus two fragments obtained in recombinant studies. Collagen triple helical domains (THDs) are labelled “Col” and α-helical coiled coils are labelled “PCoil”. Domains labelled as PfN, PCoil, PfC and Pf2 are conserved in bacteriophage and E. coli genomes. EPcIA, EPcIB, EPcIC and EPcID stand for “E. coli phage collagen-like proteins A, B, C and D”, respectively. The Col-PfC fragment is an endogenous proteolytic fragment obtained during recombinant expression of EPcIA. The PfN-PCoil fragment is a recombinant fragment produced during the biochemical study of EPcIA.

FIG. 2 shows the results of analysis by analytical ultracentrifugation (AUC) of the average molar mass of a sample of pure recombinant EPcIA (rEPcIA, sequence EPcIA-142, Table A) as a function of increasing concentration of the denaturing agent guanidinium chloride (GuHCl). Mean values (inset) are the average of three measures. In the absence of GuHCl, native rEPcIA forms trimers with an observed molecular weight of 138±6 kDa, consistent with the predicted molecular weight of a trimer. As the concentration of GuHCl increases rEPcIA denatures and the trimers dissociate into monomers; at 5 M GuHCl the observed molar mass is 43±1 kDa, which is consistent with the molecular weight of monomer rEPcIA. The trimer-to-monomer transition midpoint is estimated at around 2.5 M GuHCl. Confirmation of rEPcIA trimerisation was obtained from dynamic light scattering experiments (data not shown). Recombinant EPcIA was prepared as follows: (1) the nucleotide sequence for EPcIA was obtained by PCR amplification from a sample of genomic DNA of E. coli O157:H7 (kindly provided by C.W. Penn, University of Birmingham), using designed primers; (2) the amplified product was cloned into a protein expression vector containing poly-histidine tags and the recombinant protein was expressed using standard laboratory E. coli strains (complete amino acid and DNA sequences for rEPcIA are EPcIA-142 and EPcIA-DNA142, given in Table A and E, respectively); (3) rEPcIA was purified using nickel-affinity chromatography followed by size exclusion chromatography.

FIG. 3 shows the results of Circular Dichroism (CD) spectroscopy analysis of the Col-PfC fragment from rEPcIA (see FIG. 1). (A) The CD spectrum at 4° C. (open circles) shows the characteristic features of a collagen triple-helical structure, with a maximum of positive ellipticity at 220 nm and a deep minimum of negative ellipticity around 200 nm. These collagen features have disappeared in the spectrum at 55° C. (filled circles), indicating that the triple-helical structure has been lost at such temperature. The vertical axis represents molar ellipticity ⊖ in degrees cm² decimole⁻¹. The CD data was collected between 190 and 260 nm, with a protein concentration of 0.2 mg/ml in 10 mM Tris, 150 mM NaCl, pH 7.4. Measurements were taken in a 0.5 mm path length cell. (B) Thermal denaturation of the Col-PfC fragment monitored by CD at 220 nm (the maximum of positive ⊖ in the spectrum of Col-PfC): a sharp transition is observed at 42° C., corresponding to the decrease of ellipticity at 220 nm and loss of collagen conformation. The CD was measured as a function of increasing temperature between 4° C. and 60° C., with a protein concentration of 0.2 mg/ml in 10 mM Tris, 150 mM NaCl, pH 7.4, and a heating rate of 0.33° C./min. Trimeric Col-PfC was obtained as an endogenous proteolytic product during expression of rEPcIA and was purified from full-length rEPcIA by size exclusion chromatography.

FIG. 4 shows the molecular shape of full-length rEPcIA protein visualised by rotary shadowing electron microscopy. Inset: the rEPcIA protein has a dumbbell shape with two globular regions connected by a partially flexible stalk. This stalk contains a collagen triple helical domain (Col) next to the PfC globular region and an α-helical coiled coil region (PCoil) next to the PfN globular region. The PfN and PfC globular regions are trimeric and contain three PfN and PfC domains each.

FIG. 5 shows the results of Circular Dichroism (CD) spectroscopy analysis of rEPcIA. (A) The CD spectrum at 4° C. (open circles) is dominated by the signal of an α-helical coiled-coil structure, with two minima of negative ellipticity at 208 nm and 224 nm, respectively. The contribution of the collagen triple helical domain of rEPcIA is reflected in the pronounced local maximum of ellipticity between the two minima, at 216 nm, and the asymmetry between the two minima, the one at 208 nm being deeper. The CD spectrum changes as the temperature increases: at 45° C. (filled triangles), the spectrum maintains the characteristics of the α-helical structure, but with a significant decrease in the maximum at 215 nm and a more symmetrical appearance of the two minima, shifted to 210 nm and 222 nm, respectively; further increase of the temperature results in the disappearance of the two minima and a reduction of the overall negative ellipticity at 55° C. (filled circles), indicating loss of the α-helical coiled coil conformation. The vertical axis represents molar ellipticity β in degrees cm² decimole⁻¹. The CD data was collected between 190 and 260 nm, with a protein concentration of 0.3 mg/ml in 10 mM Tris, 150 mM NaCl, pH 7.4. Measurements were taken in a 0.5 mm path length cell. (B) The thermal denaturation of EPcIA, followed by CD at 216 nm (the maximum between the two minima at 208 nm and 224 nm), shows two transitions: a first transition at 42° C., with decrease in ellipticity, corresponds to the loss of the collagen triple-helical structure and is consistent with the observations on the denaturation of the Col-PfC fragment at the same temperature; a second, sharp transition at 52° C. with a large increase in ellipticity, corresponds to the loss of the α-helical coiled-coil structure of the PCoil and PfN domains. The CD was measured as a function of increasing temperature between 20° C. and 75° C., with a protein concentration of 0.3 mg/ml in 10 mM Tris, 150 mM NaCl, pH 7.4, and a heating rate of 0.33° C./min.

FIG. 6 shows the molecular shape of the Col-PfC fragment visualised by rotary shadowing electron microscopy. Inset: the Col-PfC has one globular PfC region followed by a rigid stalk containing the collagen triple-helical domain (Col). The region N-terminal to the collagen triple helix (to the left) can be seen as partially unstructured.

FIG. 7 shows examples of domain structures of class 1 fusion proteins within the context of the present invention. A human collagen triple helical domain sequence (hCol, shown as a grey box in both examples) is fused in frame with one or more prokaryotic or viral trimerisation domains (PVTDs), wherein said human triple helical domain and PVTDs do not naturally form part of the same protein. (A) The hCol domain replaces the Col domain from a bacterial or viral protein with EPcIA architecture. (B) A longer hCol domain replaces the tandem of Col-Pf2-Col domains from a bacterial or viral protein with EPcIB architecture. In both cases three PVTDs are kept flanking the sequence of the hCol domains.

FIG. 8 shows the domain structure of a class 2 fusion protein within the context of the present invention. A human collagen triple helical domain sequence (hCol, shown as a grey box) is fused in frame with one or more prokaryotic or viral trimerisation domains (PVTDs), and one or more triple helical domains from bacterial or viral origin, wherein said human collagen and the bacterial and viral domains do not naturally form part of the same protein. The prokaryotic or viral Col domains flanking the hCol domain can be partial fragments of the original Col domain or they can be obtained from other bacterial or viral sequences.

FIG. 9 shows examples of domain structures of class 3 fusion proteins within the context of the present invention. Designed collagen triple helical domain sequences are built from the fusion in frame of several prokaryotic or viral collagen triple helical domains, which can be identical (A) or different (B) and can be obtained from the same (A) or different (B) prokaryotic or viral collagen-like proteins. The extended triple helical domain sequences are in turn fused in frame with one or more prokaryotic or viral trimerisation domains (PVTDs), wherein the resulting fusion proteins are not identical to naturally occurring proteins.

FIG. 10 shows examples of different domain architectures of possible fusion proteins within the context of the present invention. In class I fusion proteins (A), one or more eukaryotic triple helical domains (e.g. human or animal sequences, shown as grey boxes), are fused in frame with different combinations of PVTDs. In class II fusion proteins (B), triple helical domains made of combinations of sequences from eukaryotic (e.g. human or animal) and prokaryotic or viral origin are fused in frame with different PVTDs. In class III fusion proteins (C), newly designed triple helical domains are built from sequences of several prokaryotic or viral collagen triple helical domains, which can be identical or different and from the same or different original sequence. The designed triple helical domain sequences are fused in frame with different combinations of PVTDs.

FIG. 11 shows schematically the domain architecture of three class 1 fusion proteins (recombinant hybrids, RCH) used in the examples that illustrate the present invention. Amino acid sequences for the three RCH proteins are given in Table W (RCH-1 to RCH-3) and DNA coding sequences are given in Table W (RCHDNA-1 to RCHDNA-3). Each RCH is built from the combination in frame of several domains, their sequences identified numerically (e.g. PfN-28, PfC-61). Amino acid sequences for the different PfN, PCoil and PfC domains are given in Tables H, I and J; DNA sequences for the same domains are given in Figures M to R. The human collagen THDs in these examples are different fragments of the human collagen sequence hCol-03 (the THD of collagen α1(II) chain, Table K); each fragment is identified by its residue numbers in the hCol-03 sequence. Black stars indicate natural integrin binding sites with GFPGER sequence. The white star in RCH-2 indicates a second, engineered GFPGER integrin-binding site.

FIG. 12 shows an analysis by SDS-PAGE (10%) of the expression of RCH-3 in E. coli cells. Protein bands are stained with Coomassie Brilliant Blue. Lane labels: M, molecular weight markers, in kDa; Un, uninduced sample; In, sample induced with 0.1 mM IPTG at 12° C. for 93 hours; Ly, lysate of induced sample after sonication; So, soluble fraction; In, insoluble fraction. The RCH-3 protein band migrates slower than expected, at approximately 60 kDa, a characteristic feature of collagen-like proteins. RCH-3 is expressed predominantly in the soluble fraction.

FIG. 13 shows the structural organisation of the RCH-1 protein visualised by rotary shadowing electron microscopy. The molecular shape of RCH-1 is identical to that of the EPcIA protein (FIG. 4): a dumbbell shape with two globular regions connected by a partially flexible stalk. The stalk contains the collagen THD fragment next to the PfC globular region and an α-helical coiled-coil region (PCoil) next to the PfN globular region. The PfN and PfC globular regions are trimeric and contain three PfN and PfC domains each.

FIG. 14 shows the structural organisation of the RCH-2 protein visualised by rotary shadowing electron microscopy. The molecular shape of RCH-2 is similar to that of the RCH-1 protein (FIG. 13), but with a much longer stalk due to the larger collagen THD fragment (360 residues in RCH-2 for 111 residues in RCH-1).

FIG. 15 shows the structural organisation of the RCH-3 protein visualised by rotary shadowing electron microscopy. The molecular shape of RCH-1 is similar to that of the RCH-1 protein (FIG. 13), with two globular regions joined by a partially flexible stalk, which contains the human collagen THD fragment. Each molecule shows one of the globular regions more clearly defined than the other one. This sample corresponds to the low molecular weight fraction of RCH-3, which has a significantly lower concentration of protein.

FIG. 16 illustrates the formation of dendrimer-like structures by RCHs via association of PVTDs. (A): Detail of an electron micrograph of RCH-3 molecules showing self-associated structures; the central aggregated cores appear to form by association of the PfC domains. The majority of RCH-3 molecules associate in this way generating large molecular weight structures. (B): Detail of an electron micrograph of RCH-1 molecules showing a similar self-associated structure; molecules associate through their PfC domains forming a ring-like core from which the collagen THDs and the PCoil-PfN domains radiate. Formation of such structures by RCH-1 is rare, but association of few molecules through their PfC domains is more common.

FIG. 17 shows the CD spectrum of RCH-1 at 4° C. The spectrum is similar to that of the bacterial collagen-like protein rEPcIA (FIG. 5A), and results from the combination of the signals of the collagen THD and the α-helical coiled-coil structure of the PCoil domain. The contribution of the collagen THD is reflected in the hump around 218 nm and the asymmetry between the α-helical minima at 208 nm and 222 nm (the former being much deeper).

FIG. 18 shows the thermal denaturation of RCH-1 followed by CD at 222 nm. Two transitions are observed: a first transition, with decrease in ellipticity and midpoint at 33° C., corresponds to the loss of triple-helical structure from the collagen THD; a second transition at 53° C., with a large increase in ellipticity, corresponds to the loss of the α-helical coiled-coil structure from the PCoil domain.

FIG. 19 shows the CD spectrum of RCH-2 at 4° C. The spectrum is similar to those of rEPcIA (FIG. 5A) and RCH-1 (FIG. 17), but in this case there is less α-helical coiled-coil contribution, probably due to the differences in the sequences of the PfN and PCoil domains from RCH-1 and RCH-2 (FIG. 11). The contribution of the collagen THD is reflected in the hump around 220 nm and the deep minimum at 203 nm.

FIG. 20 shows the thermal denaturation of RCH-2 followed by CD at 220 nm. As in the case of RCH-1 (FIG. 18), two transitions are observed: a first transition around 32° C., with decrease in ellipticity, corresponds to the loss of triple-helical structure from the collagen THD; a second transition at 41° C., with a large increase in ellipticity, corresponds to the loss of the α-helical coiled-coil structure from the PCoil domain.

FIG. 21 shows the spreading of HT1080 cells on RCH-3. (A) Negative control: HT1080 cells plated directly on plastic show a rounded morphology and do not spread. (B) HT1080 cells plated on plastic coverslips coated with 10 μg/ml RCH-3 show evidence of spreading. (C) Positive control: HT1080 cells plated on plastic coated with rat tail collagen (2 μg/ml). Cells were fixed after 90 minutes spreading at 37° C.

FIG. 22 shows the spreading of HT1080 cells on RCH-1 at different concentrations: (A) 20 μg/ml; (B) 30 μg/ml; (C) 50 μg/ml. Cells were fixed after being allowed to spread for 90 minutes at 37° C. on plastic coverslips coated with RCH-1.

FIG. 23 shows the percentage of spreading of HT1080 cells on surfaces coated with rat-tail collagen (filled squares) and RCH-3 (open circles) at different protein concentrations.

FIG. 24 shows schematically the domain architecture of the RCH-4 fusion protein. The amino acid sequence RCH-4 and the DNA coding sequence RCHDNA-4 are given below. RCH-4 is built from the combination in frame of two domains: PfN-15 and a THD containing residues 400-651 from hCol-03. The amino acid sequence for PfN-15 is given in Table H, and its DNA sequence is given in Tables M and N. The human collagen sequence hCol-03 is given in Table K. The black star indicates a natural integrin-binding site with GFPGER sequence.

FIG. 25 shows the CD spectrum RCH-4 at 4° C. The spectrum is very similar to that of a collagen THD, with a hump around 218 nm and a deep minimum at 195 nm.

Table A shows the amino acid sequences of EPcIA proteins. Each sequence is identified with a unique EPcIA-nnn code (EPcIA-001 to EPcIA-142), as well as its UniProt sequence identifier. Sequence EPcIA-142 corresponds to the recombinant construct rEPcIA used in biochemical studies.

Table B shows the amino acid sequences of EPcIB proteins. Each sequence is identified with a unique EPcIB-nnn code (EPcIB-001 to EPcIB-021), as well as its UniProt sequence identifier.

Table C shows the amino acid sequences of EPcIC proteins. Each sequence is identified with a unique EPcIC-nnn code (EPcIC-001 to EPcIC-005), as well as its UniProt sequence identifier.

Table D shows the amino acid sequence of EPcID proteins. Only one sequence is known to date, EPcID-001. Its UniProt sequence identifier is also provided.

Table E shows the DNA sequences of EPcIA proteins. Each sequence is identified with a unique EPcIA-DNAnnn code (EPcIA-DNA001 to EPcIA-DNA142), as well as its UniProt and genome sequence identifiers (EMBL/GenBank). Sequence EPcIA-DNA142 corresponds to the recombinant construct rEPcIA used in biochemical studies.

Table F shows the DNA sequences of EPcIB proteins. Each sequence is identified with a unique EPcIB-DNAnnn code (EPcIB-DNA001 to EPcIB-DNA021), as well as its UniProt and EMBL/GenBank sequence identifiers.

Table G shows the DNA sequences of EPcIC and EPcID proteins. Each sequence is identified with a unique EPcIC/D-DNAnnn code (EPcIC-DNA001 to EPcIC-DNA005; EPcID-DNA001), as well as its UniProt and EMBL/GenBank sequence identifiers.

Table H shows a non-redundant set of amino acid sequences of PfN capping domains from prokaryotic and viral collagen-like proteins. Each sequence is identified with a unique PfN-nn code (PfN-01 to PfN-86).

Table I shows a non-redundant set of amino acid sequences of PCoil capping domains from prokaryotic and viral collagen-like proteins. Each sequence is identified with a unique PCoil-nn code (PCoil-01 to PCoil-46).

Table J shows a non-redundant set of amino acid sequences of PfC capping domains from prokaryotic and viral collagen-like proteins. Each sequence is identified with a unique PfC-nnn code (PfC-01 to PfC-61).

Table K shows the amino acid sequences of the THD domains from human collagens. Each sequence is identified with a unique hCol-nn code (hCol-01 to hCol-49), as well as its UniProt sequence identifier.

Table L shows the amino acid sequences of the THD domains from human collagen-like proteins. Each sequence is identified with a unique hCol-nn code (hCol-50 to hCol-89), as well as its UniProt sequence identifier.

Table M shows non-degenerate DNA sequences for the PfN capping domains from Table H, obtained using the most likely codons for expression in E. coli. Each sequence is identified with a unique PfN-DNAnn code (PfN-DNA01 to PfN-DNA86).

Table N shows degenerate DNA sequences for the PfN capping domains from Table H, using a consensus IUPAC/IUB notation sequence derived from all possible codons for each amino acid (NC-IUB (1985) Biochem. J. 229: 281-286). Each sequence is identified with a unique PfN-CNAnn code (PfN-CNA01 to PfN-CNA86).

Table O shows non-degenerate DNA sequences for the PCoil capping domains from Table I, obtained using the most likely codons for expression in E. coli. Each sequence is identified with a unique PCoil-DNAnn code (PCoil-DNA01 to PCoil-DNA46).

Table P shows degenerate DNA sequences for the PCoil capping domains from Table I, using the same consensus IUPAC/IUB notation sequence as in Table N. Each sequence is identified with a unique PCoil-CNAnn code (PCoil-CNA01 to PCoil-CNA46).

Table Q shows non-degenerate DNA sequences for the PfC capping domains from Table J, obtained using the most likely codons for expression in E. coli. Each sequence is identified with a unique PfC-DNAnn code (PfC-DNA01 to PfC-DNA61).

Table R shows degenerate DNA sequences for the PfC capping domains from Table J, using the same consensus IUPAC/IUB notation sequence as in Table N. Each sequence is identified with a unique PfC-CNAnn code (PfC-CNA01 to PfC-CNA61).

Table S shows non-degenerate DNA sequences for the THD domains of human collagens (Table K), using the most likely codons for expression in E. coli. Each sequence is identified with a unique hCol-DNAnn code (hCol-DNA01 to hCol-DNA49).

Table T shows non-degenerate DNA sequences for the THD domains of human collagen-like proteins (Table L), using the most likely codons for expression in E. coli. Each sequence is identified with a unique hCol-DNAnn code (hCol-DNA50 to hCol-DNA89).

Table U shows degenerate DNA sequences for the THD domains of human collagens (Table K), using the same consensus IUPAC/IUB notation sequence as in Table N. Each sequence is identified with a unique hCol-CNAnn code (hCol-CNA01 to hCol-CNA49).

Table V shows degenerate DNA sequences for the THD domains of human collagen-like proteins (Table L), using the same consensus IUPAC/IUB notation sequence as in Table N. Each sequence is identified with a unique hCol-CNAnn code (hCol-CNA50 to hCol-CNA89).

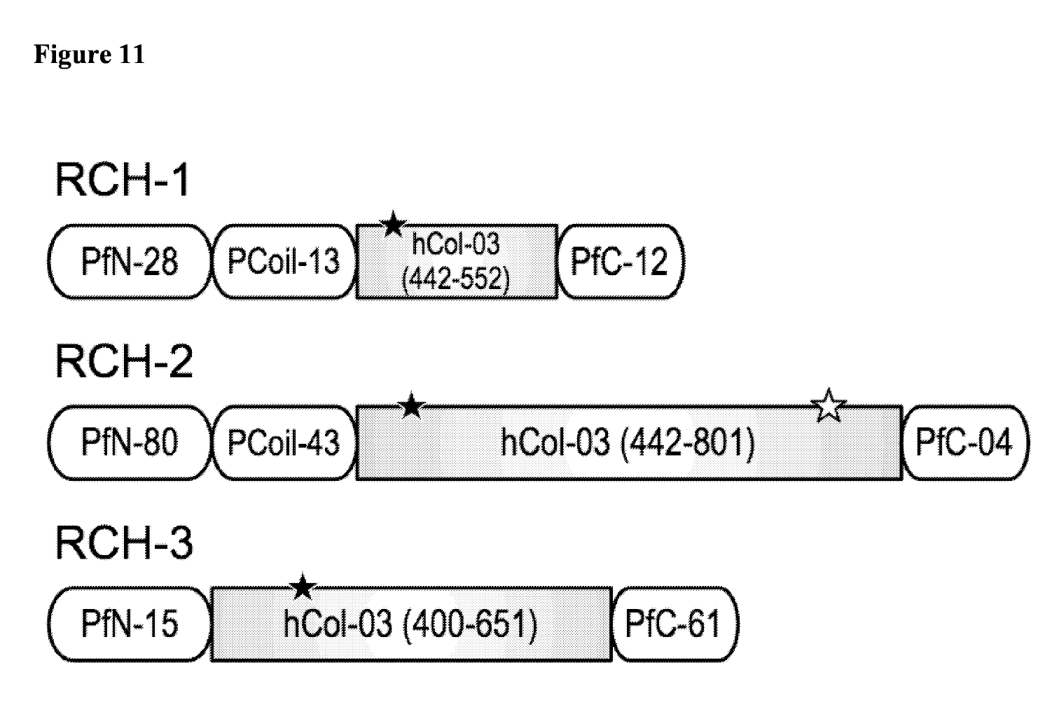

Table W shows the amino acid sequences of the fusion, recombinant collagen hybrid proteins (RCH) used in the examples provided. Each sequence is identified with a unique RCH-n code (RCH-1 to RCH-3). See FIG. 11 for the domain composition of each RCH protein. Integrin-binding sites (sequence GFPGER) are underlined on each RCH sequence. Table W also shows the DNA sequences coding for the fusion, recombinant collagen hybrid proteins (RCH) used in the examples provided. Each sequence is identified with a unique RCHDNA code (RCHDNA-1 to RCHDNA-3). The restriction digestion sites BamI (GGATCC) and EcoRI (GAATTC) restriction digestion sites are underlined on each sequence. These sites were used to clone each sequence into different protein expression vectors.

### DETAILED DESCRIPTION

Traditionally, production of mammalian collagens and gelatines in bacterial systems has had limited success due to problems of low-yield, poor solubility, and lack of stability. The present invention is based upon the discovery of the exceptional stability and solubility properties of the collagen-like proteins from bacteria, particularly E. coli, particularly E. coli O157:H7. The present invention has opened the opportunity for a high-yield production of more soluble and more stable recombinant eukaryotic collagens in prokaryotes.

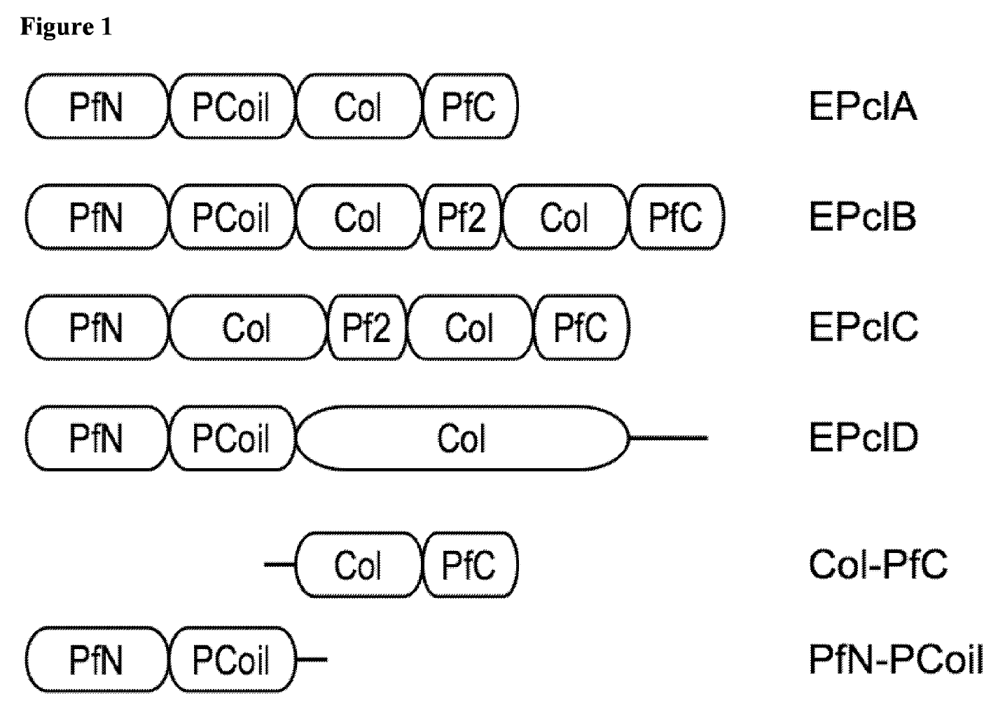

The present invention differs from the methods of the prior art in the use of PVTDs for the engineering of hybrid sequences comprising eukaryotic collagen or collagen-like domains in tandem with PVTDs. It is based on the identification of collagen-like protein sequences in the genomes of prokaryotes, such as gram negative bacteria, such as E. coli, such as strain O157:H7, and in bacteriophages or prophages infecting these strains or embedded in their genomes. These collagen-like protein sequences may be of bacteriophage origin. At least three different domain architectures have been identified (FIG. 1), in more than a hundred and sixty sequences (EPcIA-001 to EPcIA-141; EPcIB-001 to EPcIB-021; EPcIC-001 to EPcIC-005; EPcID-001), with several sequences known for each domain arrangement. Within any given domain architecture, different sequences show variability in the length of their collagen triple helical domains. These collagen-like structures share conserved domains, herein named PfN, PfC, PCoil and Pf2, which flank both sides of the collagen or collagen-like triple helical domains (FIG. 1).

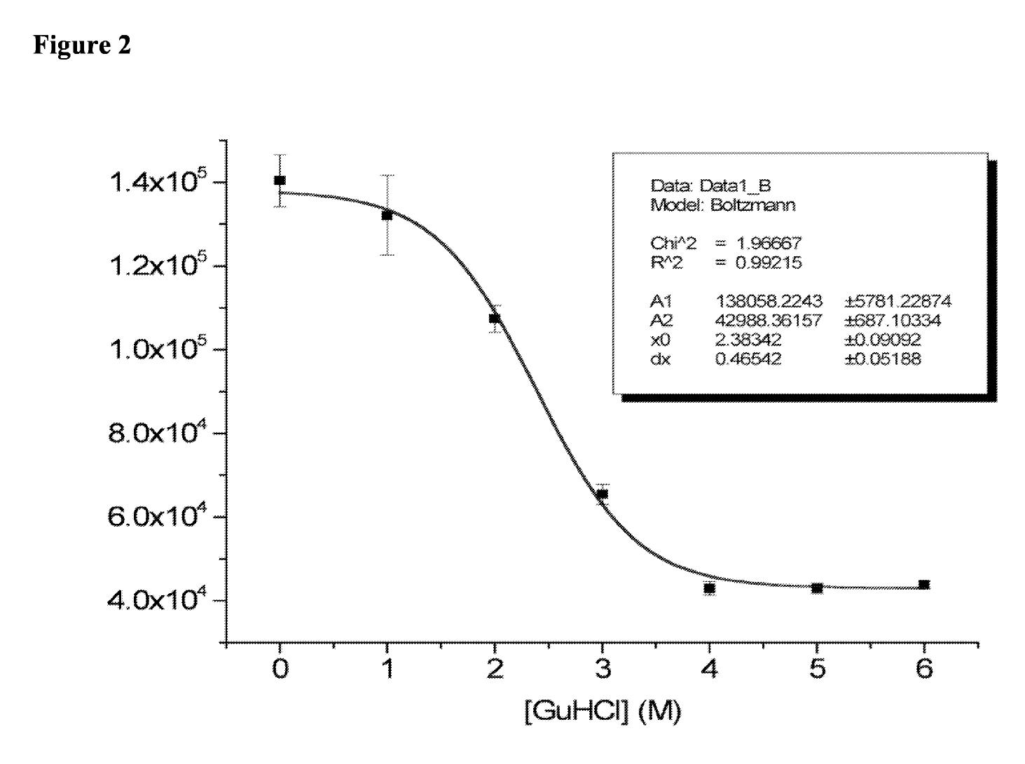

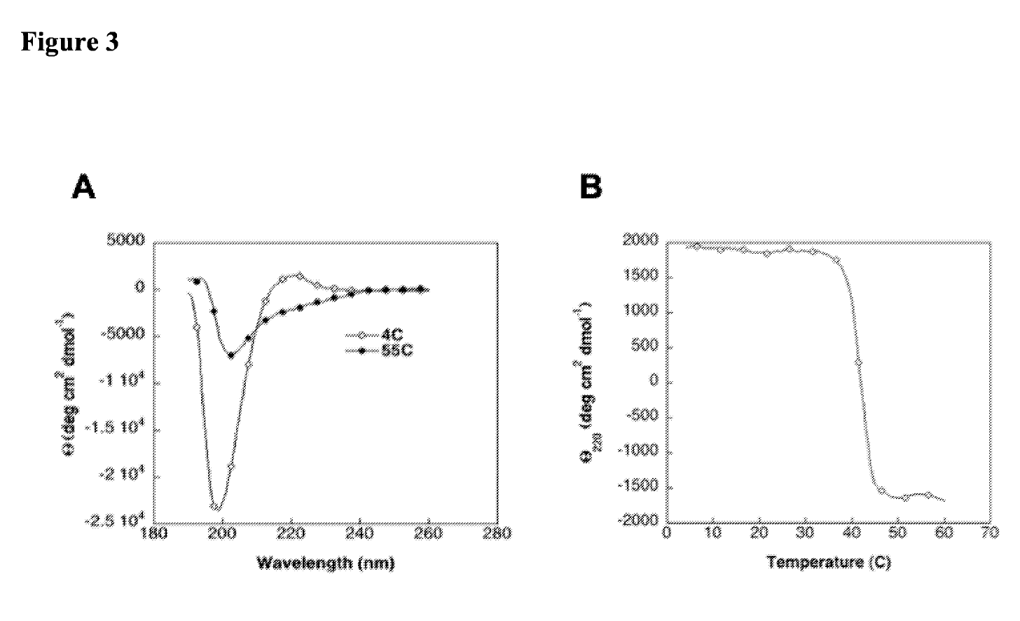

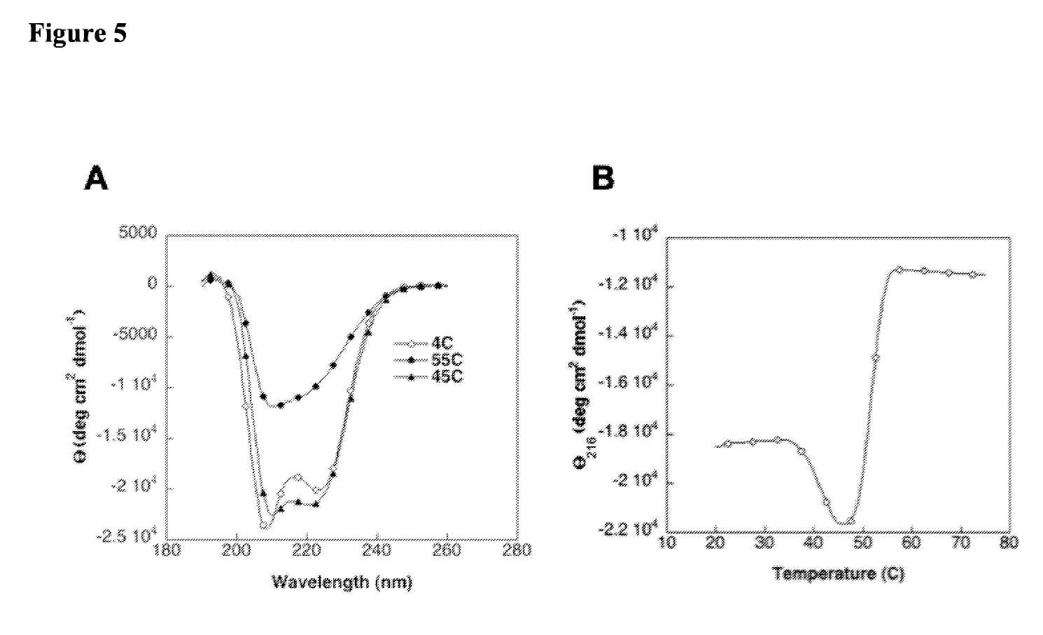

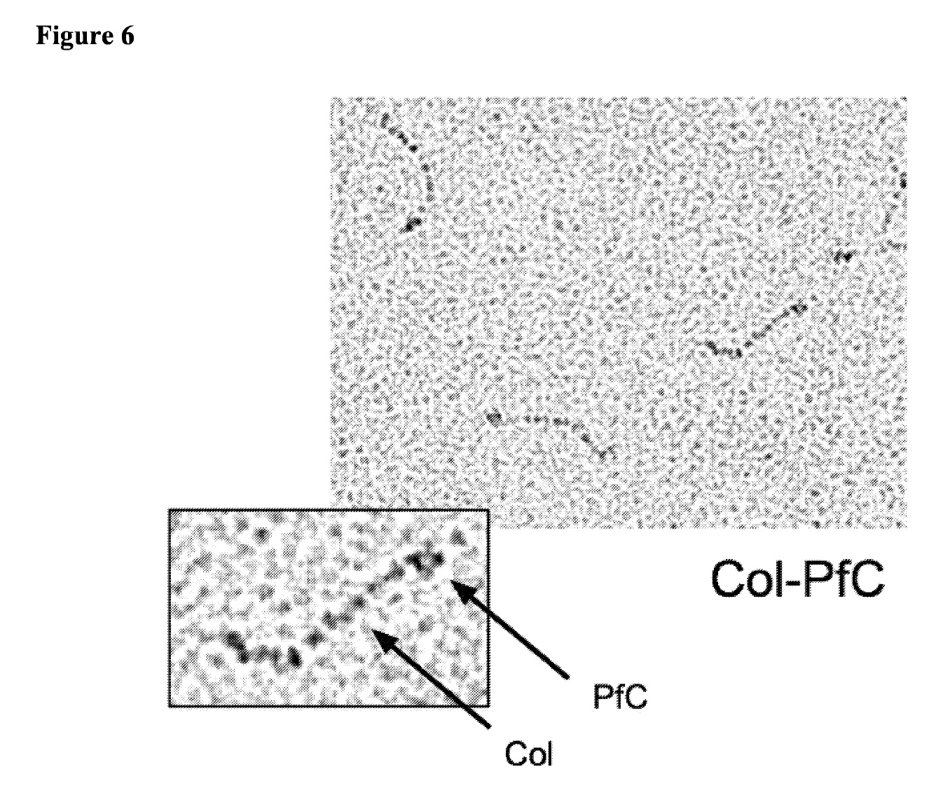

The collagen-like proteins encoded by these sequences share structural characteristics with eukaryotic collagen proteins. The EPcIA protein from the Sakai strain of E. coli O157:H7 forms trimeric assemblies (FIG. 2), which show unusually high thermal stability for a collagen triple helical domain without hydroxyproline residues. Rotary shadowing electron microscopy of EPcIA reveals a dumbbell structure (FIG. 3) where the PfN and PfC domains form globular domains that are linked by a flexible stalk made of a collagen triple helix and a very stable, trimeric α-helical coiled coil (FIG. 5).

The fusion proteins of the present invention comprising a eukaryotic collagen domain and a PVTD have the advantage of being more thermally stable, having increased solubility and being composed of polypeptide monomers which are more resistant to degradation within a host cell. Preferably, the fusion proteins of the invention exhibit one or more of the above-mentioned characteristics, preferably two or more of said characteristics.

A “fusion protein or polypeptide” within the context of the present invention means a protein or polypeptide having two or more different amino acid sequences which are not naturally found in the same protein i.e. are heterologous to each other. Specifically, the fusion protein or polypeptide of the present invention may comprise a eukaryotic collagen or collagen-like domain and a heterologous PVTD. Preferably, a fusion protein or polypeptide of the invention may comprise one or more eukaryotic collagen or collagen-like domains. More preferably, the fusion protein or polypeptide of the invention may comprise two or more eukaryotic collagen or collagen-like domains. The fusion protein or polypeptide of the invention may comprise one or more prokaryotic or viral collagen or collagen-like domains, including those which do not mediate trimerisation. Preferably, the fusion protein does not comprise prokaryotic or viral collagen or collagen-like domains. Thus, preferably, substantially all the collagen or collagen-like domains of the fusion protein or fusion polypeptide are eukaryotic.

A fusion protein of the invention is trimeric, composed of three polypeptide chains. Preferably, at least the collagen- or collagen-like domains of the polypeptide chains cooperate to form a triple helix, of a collagen-like structure (Beck et al J Structural Biol 122 17-20 1998). A part of the fusion protein of the invention may be composed of an alpha helical coiled coil structure, or alternative three dimensional structures. Each polypeptide chain may be composed of one or more fusion polypeptides, as disclosed herein, or may be composed of any combination of one or more eukaryotic collagen or collagen-like domains, PVTD's or other prokaryotic or viral domains or eukaryotic or prokaryotic or viral functional sequences. Operably linked, these polypeptides may form a polypeptide chain.

The fusion protein or polypeptide of the invention may comprise a PVTD. Herein, a PVTD is a domain which is capable of mediating trimerisation of polypeptide chains, preferably into a triple helical structure. Preferably, a PVTD is capable of maintaining a triple helical structure below the melting temperature of a collagen or collagen like domain of the polypeptide chains, and preferably is capable of maintaining the polypeptide chains as a trimer below the melting temperature of a PVTD of the fusion protein. Preferably, a PVTD is prokaryotic or viral in origin.

Herein, a PVTD may serve as a capping domain, or to mediate one or more of the functional characteristics of the fusion proteins of the invention, as defined above.

Preferably, a fusion protein or polypeptide of the invention comprises in tandem heterologous sequences from different organisms. For example, the fusion protein or polypeptide may comprise in tandem a PVTD, a eukaryotic collagen or collagen like sequence, and a second or further PVTD. Alternatively, and by way of example, a fusion protein or polypeptide of the invention may comprise a eukaryotic collagen or collagen-like domain comprising therein a PVTD, and having at one or both ends a further PVTD. It will be apparent to the skilled person that any combination of one or more sequences independently selected from the groups consisting of one or more eukaryotic collagen or collagen-like domains, one or more PVTDs, one or more eukaryotic, prokaryotic or viral functional sequences, one or more prokaryotic or viral collagen or collagen-like domains and one or more non-collagen sequences may be provided in a fusion protein or polypeptide of the invention. Preferably, heterologous sequences will be operably linked to each other, for example by peptide bonds or chemical linkage, to form a fusion protein or polypeptide.

In the fusion protein or polypeptide, a PVTD may be provided:

i) within a eukaryotic collagen or collagen-like domain; and/or
ii) flanking one or both ends of a eukaryotic collagen or collagen-like domain;
iii) within non-eukaryotic collagen or collagen-like domain of the fusion polypeptide and/or flanking one or both ends thereof.

Any combination of the above independently selected options are provided for within the scope of the present invention. Where more than one PVTD is present, all may be provided internally within the eukaryotic sequence. Alternatively, one or more PVTDs may be provided flanking a collagen or collagen-like domain. More preferably, each polypeptide chain will be flanked at one or both ends by a PVTD, such that they are able to mediate the formation of a trimeric, preferably triple helical, fusion protein.

The PVTDs in each polypeptide chain of a trimeric fusion protein may all be the same or some or all may be different. By “flanked” means positioned at one or both ends of a sequence, preferably a heterologous sequence, for example a eukaryotic collagen or collagen-like domain. It is appreciated that a PVTD must be operably linked to a sequence of the fusion protein or polypeptide, but it is not necessary for a PVTD to follow immediately from a collagen or collagen-like domain. Thus, linker, spacer, or indeed other functional sequences may be provided between a sequence, preferably a heterologous sequences, preferably a eukaryotic collagen or collagen-like domain, and a PVTD.

Preferably, any PVTD on the three polypeptide chains of a trimeric fusion protein will be positioned such that they are able to associate in such a manner that the three polypeptide chains are able to form a trimeric, and preferably a triple helical, protein. For example, PVTDs may flank one (preferably the same) or both ends of a eukaryotic collagen or collagen-like domain in all three polypeptide chains, e.g. the N terminal or C terminal end. Alternatively, where a PVTD is an internal sequence, it may all be positioned within a pre-determined number of amino acids from an end of the polypeptide chain or a collagen or collagen-like domains (eukaryotic, prokaryotic or viral). PVTDs can be used to bring together polypeptide sequences of the same or different lengths as a trimer. Where different, PVTDs will be positioned such that formation of a trimer is possible. For example, a PVTD may be provided at one end of a polypeptide chain, and internally in another chain, such that PVTDs meet by folding of the latter polypeptide chain. Preferably, PVTDs may be provided at a non-folded end of the three chains. The optimum positioning of PVTDs in polypeptide chains of different lengths can be determined by a person skilled in the art using their common general knowledge of collagen. Also envisaged is an embodiment where one or more corresponding PVTDs capable of associating with each other are provided on two of the three polypeptide chains.

In addition to PVTDs, the fusion proteins or polypeptides of the invention may further comprise one or more prokaryotic domains. These may be provided in tandem with a eukaryotic collagen or collagen-like domain, a PVTD, a functional sequence, or any other part of the fusion polypeptide. Such a prokaryotic domain may be provided or flanking within one of the afore-mentioned eukaryotic or PVTD sequences. Such a prokaryotic domain will preferably be collagen-derived. Such a prokaryotic domain may be any functional sequence, including, for example, stabilization sequences, binding sites, cysteine cross links, cleavage sites, linkage sites, and indeed any other suitable sites which may provide desirable functionalities in the fusion protein. The prokaryotic domain may be naturally occurring, or a fragment, derivative, variant or modified version of a naturally occurring prokaryotic domain. In this embodiment, the terms naturally occurring, fragments, derivatives, variants, and modified are as defined above in relation to eukaryotic collagen or collagen-like domains and PVTDs. Such prokaryotic domains will preferably be operably linked to the eukaryotic collagen or collagen-like domain and/or other prokaryotic sequences and/or PVTDs. Where more than one prokaryotic domain is provided in a fusion protein or polypeptide of the invention, one or more of these may be independently selected from the groups consisting of stabilization sequences, binding sites, cysteine cross links, cleavage sites, linkage sites, and indeed any other suitable sites which may provide desirable functionalities in the fusion protein.

The fusion protein or polypeptide of the invention may comprise one or more non-collagen domains. Such non-collagen domains do not contain the repetitive Gly-X-Y amino acid sequence defined above, and/or do not have the ability to form a trimer or triple helical domain.

In a preferred embodiment of the present invention, the eukaryotic collagen or collagen-like domain sequence, any prokaryotic or viral collagen or collagen-like domain, and/or one or both PVTDs may be engineered to comprise non-native sequences. For example, a human collagen or collagen-like domain present in a fusion polypeptide or protein of the first aspect of the invention may have been engineered to contain non-native integrin binding sties, or non-native binding sites for other receptors or other collagen-binding proteins from the extracellular matrix or elsewhere. In another example, one or more of the PVTDs from one or more fusion polypeptides or proteins of the invention may have been engineered to promote heterotrimeric associations rather than homotrimeric ones.

The triple helical fusion protein may be a homotrimer, or a heterotrimer. In a homotrimer, the three polypeptide chains making up the triple helix are identical, in terms of sequence. In a heterotrimer, two or more of the three polypeptide chains are non-identical in terms of sequence. In both homotrimers and heterotrimers, the one or more prokaryotic or viral sequences in two or more of the three polypeptide chains may be the same or different. The three polypeptide chains may be the same or different in length. Preferably, the three polypeptide chains making up a triple helical protein will be substantially the same length, or at least any difference in length of the triple helical region is less than 70%, 60%, 50%, 40%, 30%, 20% or 10% compared to one or both of the triple helical regions from the remaining chains in the helix.

Preferably, in a homotrimer where PVTDs are provided within the eukaryotic collagen or collagen-like domain, these will be substantially the same in all three polypeptide chains, except where it may be functionally desirable for part of one of the polypeptide chains to be heterotrimeric, for example for steric reasons to form an exposed binding site or cleavage site. Where PVTDs are provided at one or both ends of the eukaryotic collagen or collagen-like domain, these may the same or different between two or more of the polypeptide chains of the invention, in homotrimers or heterotrimers, as long as trimerisation of the three polypeptide chains remains possible. Preferably, the PVTDs which are intended to cooperate with each other on the three polypeptide chains will be the same.

It is envisaged that any number and combination of PVTDs may be provided in any one fusion polypeptide or protein, with any number and combination of eukaryotic collagen or collagen-like domains. Thus, any one, two, three, four, five, six, seven, eight, nine, ten or more independently selected PVTDs may be provided in combination with any one, two, three, four, five, six, seven, eight, nine or ten or more independently selected eukaryotic collagen sequences. To avoid lengthy recitation of preferred embodiments, the present invention expressly provides for fusion proteins or fusion polypeptides comprising

a) one or more PVTD independently selected from
i) a PVTD of any of EPcIA-001 to EPcIA-142 of Table A, any of EPcIB-001 to EPcIB-021 of Table B, any of EPcIC-001 to EPcIC-005 of Table C, or EPcID-001 of Table D, any of PfN-01 to PfN-86 of Table H, any of PCoil-01 to PCoil-46 of Table I, any of PfC-01 to PfC-61 of Table J, and a Pf2 sequence, preferably one of the Pf2 domains in sequences any of EPcIB-001 to EPcIB-021 of Table B;
ii) having an amino acid sequence having at least 50%, 60%, 70%, 80%, 90%, 91%, 92%, 93%, 94%, 95%, 96%, 97%, 98% or 99% sequence identity with a PVTD of i); or
iii) encoded by a nucleic acid selected from the group consisting of sequences of Tables E to G and M to R or a nucleic acid sequence having at least 50%, 60%, 70%, 80%, 90%, 91%, 92%, 93%, 94%, 95%, 96%, 97%, 98% or 99% sequence thereto, or
iv) a fragment or derivative of an afore-mentioned sequence which functions as a PVTD
   b) one or more eukaryotic collagen or collagen-like domains independently selected from
i) a human fibrillar collagen chain selected from α1(I), 2(1), α1(II) and α1(III);
ii) a eukaryotic collagen or collagen-like domain comprising a sequence selected from the group consisting of sequences hCol-01 to hCol-89 of Table K and L, or
iii) a sequence consisting of a sequence selected from the groups consisting of the human collagen sequences any of hCol-01 to hCol-49 of Table K and the collagen-like domains of any of hCol-50 to hCol-89 of Table L;
iv) a domain or sequence having at least 50%, 60%, 70%, 80%, 90%, 91%, 92%, 93%, 94%, 95%, 96%, 97%, 98% or 99% sequence identity with a sequence of i) ii) or iii);
v) fragments, variants or derivatives of a sequence of any of i) to iv).

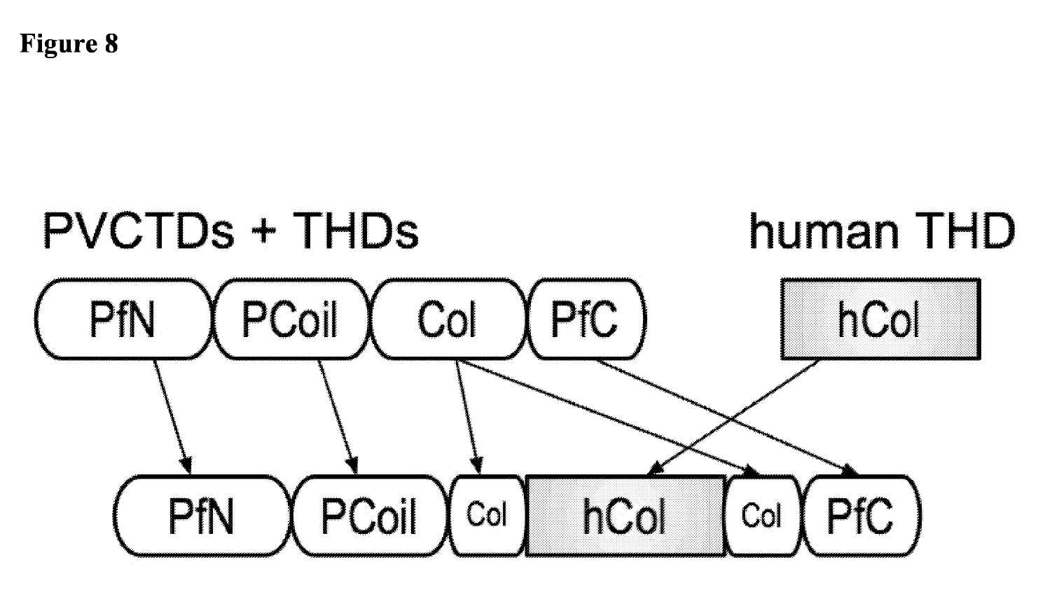

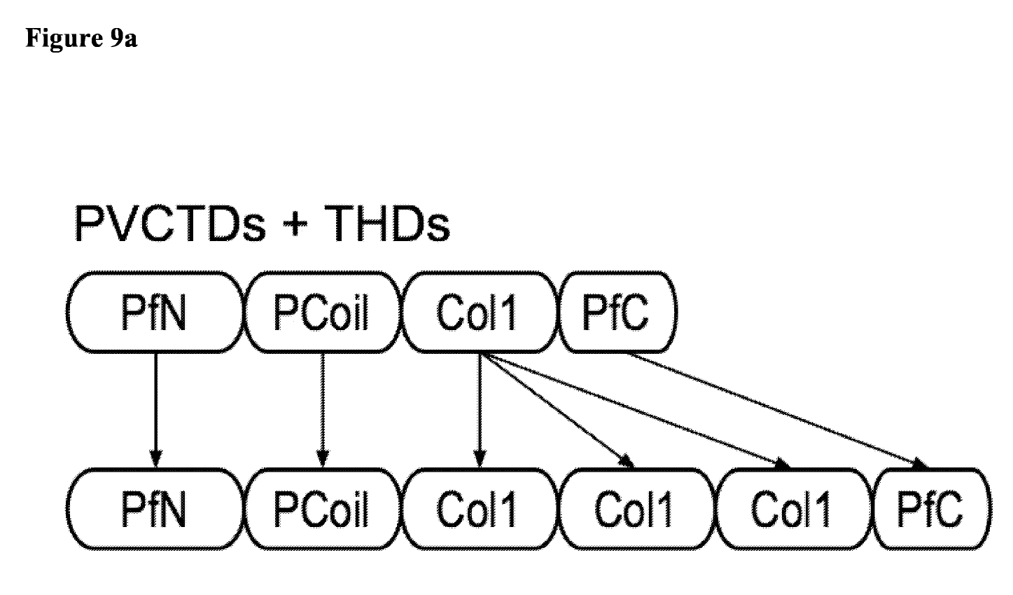

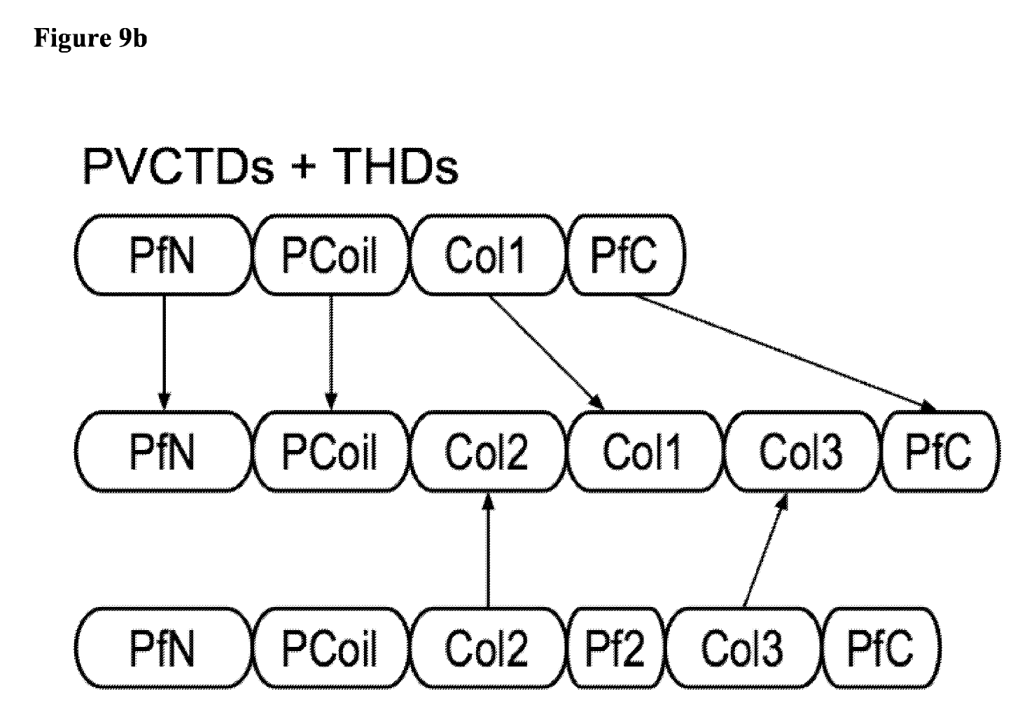

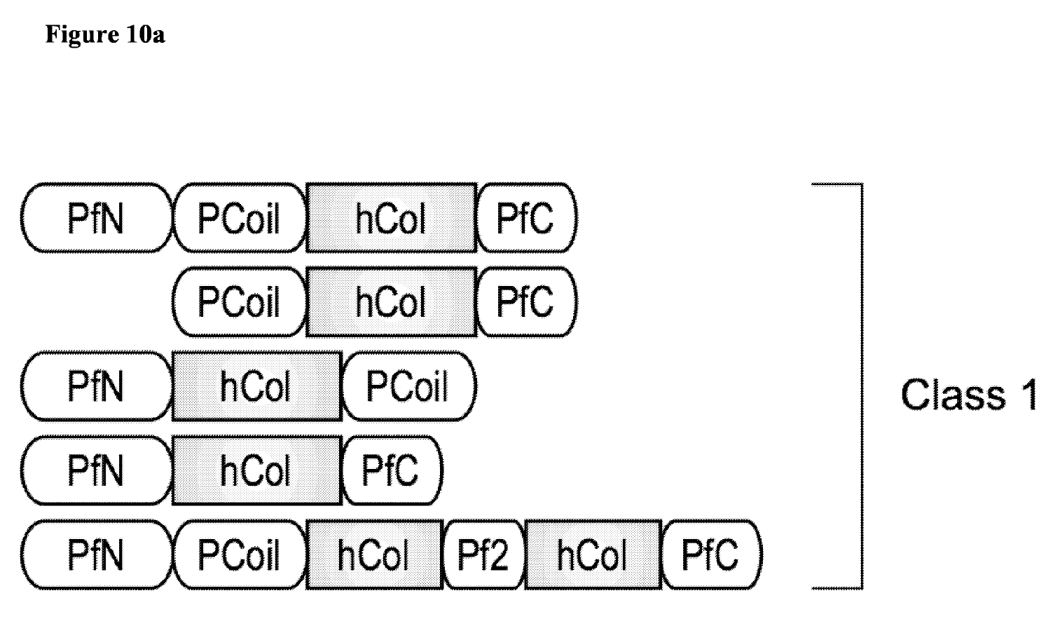

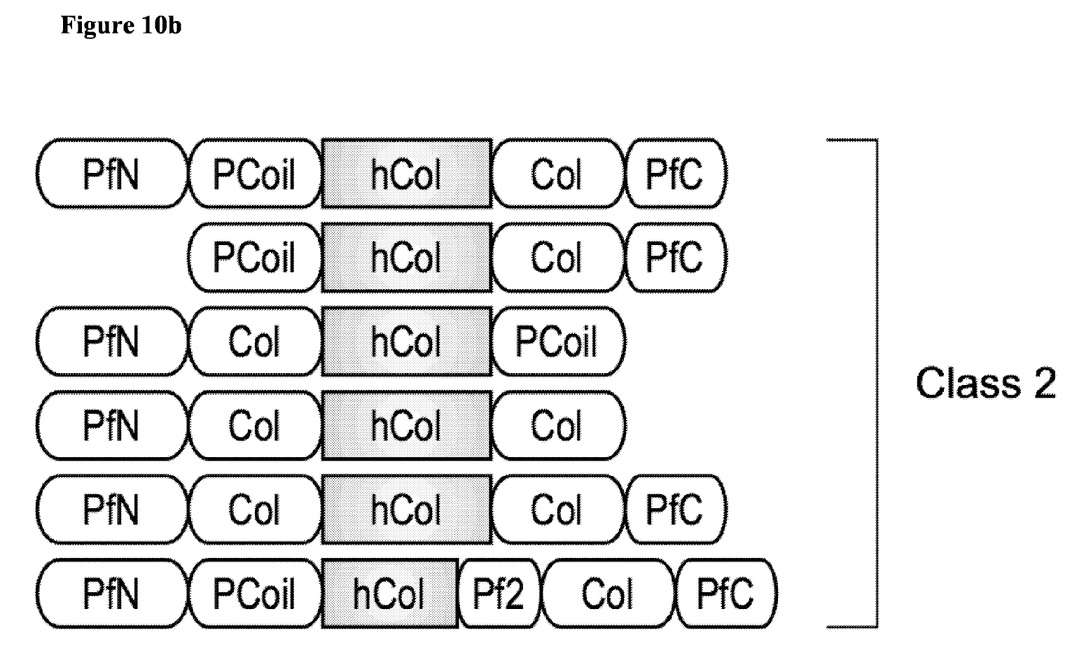

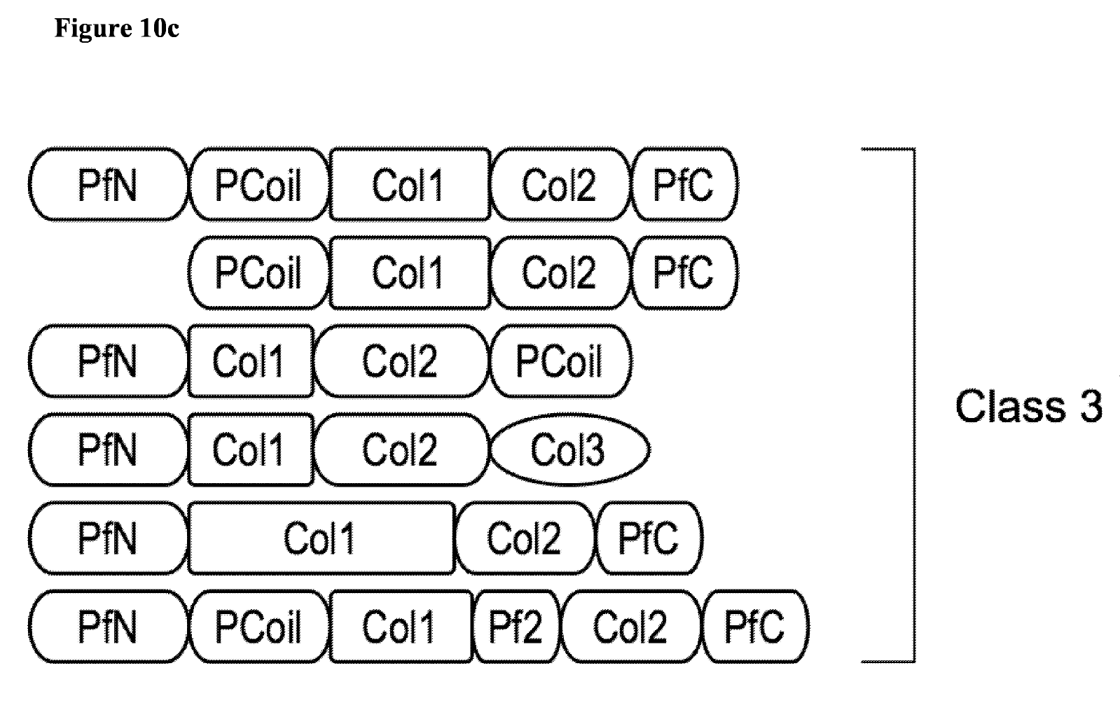

It will be appreciated that each and every combination of one or more eukaryotic collagen or collagen-like domain and one or more PVTD is provided by the present invention, which is not limited to the specific examples provided herein. Thus, any one or more of the above mentioned sequences may be provided as a fusion protein or polypeptide with any one or more of the above mentioned sequences. However, examples of preferred fusion polypeptides of the present invention are provided in FIGS. 1, 7, 8, 9, 10 and 11, and RCH 1 to 3 of the Examples.

In a preferred embodiment, the present invention provides a eukaryotic collagen or collagen-like domain wherein only one end of the eukaryotic domain is flanked by a PVTD. Preferably, the PVTD is one which serves as a capping domain.

A fusion protein or polypeptide of the invention may be polymerized or linked to a peptide or non-peptide coupling partner such as, but not limited to, an elongation factor, a stabilization factor, an effector molecule, a label, a marker, a drug, a toxin, a carrier or transport molecule or a targeting molecule such as an antibody or binding fragment thereof or other ligand. A preferred elongation factor is the prokaryotic protein, NusA. A preferred purification tag is GST. Techniques for coupling proteins to both peptide and non-peptide coupling partners are well-known in the art, and include recombinant DNA technology such that where the coupling partner is a protein, it may be expressed in-frame with the fusion polypeptide or protein.

The fusion protein or polypeptide may be crosslinked by thermal dehydration, chemical, and/or light treatment. Techniques for cross-linking proteins are well-known to those of skill in the art.

In addition, the fusion protein or polypeptide may undergo post-translational modifications. Such modifications include, but are not limited to, acetylation, carboxylation, glycosylation, phosphorylation, lipidation and acylation. Post-translational processing which cleaves a precursor form into a mature form of the protein may also be important for correct insertion, folding and/or function.

Herein, the terms “collagen” or “collagen-like” refer to proteins or polypeptide chains which comprise Gly-X-Y triplet sequences with a minimum of three triplets in any of its three registers (that is . . . Gly-X-Y-Gly-X-Y-Gly-X-Y . . . , . . . Y-Gly-X-Y-Gly-X-Y-Gly-X . . . , or . . . X-Y-Gly-X-Y-Gly-X-Y-Gly . . . ), independently of the polypeptides forming trimers or proteins forming a triple helical structure or not. Thus, the definition of collagen or collagen-like domains refers to the occurrence of the repetitive sequence at the primary structure level, and bears no implications for the actual secondary, tertiary or quaternary structures of the polypeptide or protein containing it. This particular sequence enables collagen to form its characteristic triple-helical structure. The term “triplet” refers to a set of three amino acids as defined by the set Gly-X-Y, wherein X and Y can be any amino acid. In the present invention, the term “collagen” includes naturally occurring collagen, and fragments, domains, derivatives, mimetics, variants and chemically modified compounds of said naturally occurring collagen. Preferably, the eukaryotic collagen or collagen-like domain of the invention will be capable of mediating one or more collagen activities, such as being able to bind to cell surface molecules such as integrin or fibronectin, or glycoproteins or proteoglycans, or will be derived from a eukaryotic collagen protein which is capable of mediating one or more such activities.

All human, mammalian, vertebrate and metazoan collagen types contain one or more THDs (triple helical domains) that are often flanked and/or separated by non-collagen domains (often referred in the literature as NC domains). Additionally, human, mammalian, vertebrate and metazoan genomes show instances of collagen-like proteins not formally identified as collagens at present but that contain one or more instances of triple helical domains. Additionally, many putative proteins containing triple helical domains in their primary sequence have been identified in prokaryotic and viral genomes. These proteins are usually referred to as “collagen-like proteins”. Collagen may be distinguished from collagen-like proteins because the three polypeptide chains are staggered, such that at least at one end of the protein the three chains are not the same length.

Although the present invention is described with reference to type I collagen, which is the most commonly used collagen in industry, the term “collagen” as used herein refers to any one of the known collagen types, including collagen types I through XXIX, as well as to any other collagens, and prokaryotic or eukaryotic.

A fragment of a collagen or collagen-like protein, for use in the present invention, preferably comprises a repetitive Gly-X-Y amino acid sequence. It may be a single chain polypeptide or may form a trimer and more preferably a characteristic collagen triple helical structure under suitable temperature, pH or solvent conditions. In the present invention, a fragment may include three or more triplets, in any of its three registers (for example . . . Gly-X-Y-Gly-X-Y-Gly-X-Y . . . , . . . Y-Gly-X-Y-Gly-X-Y-Gly-X . . . , or . . . X-Y-Gly-X-Y-Gly-X-Y-Gly . . . ). Fragments of collagen or collagen-like proteins or polypeptides of the invention have no maximum length. They may have a defined minimum or maximum length. In the present invention, the fragments may be uninterrupted. Alternatively, they may additionally comprise naturally occurring interruptions or engineered interruptions in the repetitive sequence. The interruptions may range from one to several amino acids, and may affect the function of the fragment. Fragments of the present invention may be capable of mediating one or more functions of naturally occurring collagen, such as being able to bind to cell surface molecules such as integrin or fibronectin, other collagen receptors, other collagen-binding proteins, nucleic acids, sugars and polysaccharides, glycoproteins, proteoglycans, lipids, lipoproteins, metals, inorganic salts, or mineral crystals. Preferably, a fragment may comprise one or more specific domains of the naturally occurring sequence, for example domains having a desired functionality.

A collagen or collagen-like polypeptide chain will preferably have a helical structure. The helix may be right handed or left-handed preferably the latter, and preferably will have the ability to form trimers and most preferably triple helical structures with two other collagen or collagen-like polypeptide chains. A collagen or collagen-like protein will typically be a trimer, and more preferably will have a triple helical structure. Thus, the term “triple helical” in relation to collagen will be well understood by persons skilled in the art to mean twisted together to form a coiled coil structure, either right or left handed. The collagen proteins referred to herein will preferably have the ability to form super-coiled-coil structures, micro-fibrillar and fibrillar structures, or network or mesh, or any other supramolecular structures similar to those observed in different collagen types in humans or animals.

A eukaryotic collagen or collagen-like domain of the fusion protein or polypeptide will be derived from invertebrate or vertebrate collagen or collagen-like proteins. Preferably, vertebrate sources include mammalian, ruminate, fish or human. The eukaryotic collagen or collagen-like domain of the fusion protein of polypeptide may be non-chimeric or chimeric, such that it is composed of two or more heterologous collagen or collagen-like domains, from different proteins, operably linked to form a single collagen or collagen-like domain. The different collagen or collagen-like domains within the chimeric collagen or collagen-like domain of the fusion protein or polypeptide may be independently selected from the group consisting of invertebrate or vertebrate sources, for example mammalian, ruminate, fish, or human collagen or collagen-like proteins. In any one fusion protein or polypeptide of the invention, where more than one eukaryotic collagen or collagen-like domains are present, all may non-chimeric, or alternatively one or more may be chimeric. Where more than one eukaryotic collagen or collagen-like domains are present, one or more of these may be independently selected from invertebrate or vertebrate, for example from the groups consisting of mammalian, ruminate, fish and human domains.

Preferably, a eukaryotic collagen or collagen-like domain may comprise a human fibrillar collagen chain selected from α1(I), 2(I), α1(II) and α1(III), or a fragment or derivative thereof. Most preferably, a eukaryotic collagen or collagen-like domain of the fusion protein or polypeptide may comprise a sequence selected from the group consisting of sequences hCol-01 to hCol-89 of Table K and L. Where more than eukaryotic collagen or collagen-like domains are present in the fusion protein or polypeptide, one or more of these may independently comprise a sequence selected from the groups consisting of the human collagen sequences hCol-01 to hCol-49 of Table K and the collagen-like domains of hCol-50 to hCol-89 of Table L, or variants or derivatives thereof, or fragments thereof. SwissProt/Uniprot accession codes for the above-mentioned human collagen chains are provided in Table K and L (for example P02452 for the human α1(I) chain; P08123 for the human α2(I) chain; P02458 for the α1(II) chain; P02461 for the human α1(III) chain; etc). Derivatives or variants are sequences which share at least 60%, preferably 70%, 75%, 80%, 85%, 90%, 91%, 92%, 93%, 94%, 95%, 96%, 97%, 98%, or 99% sequence identity with one or more of the above human fibrillar collagen chains or fragments thereof, of a human collagen or collagen-like domain as defined by one or more sequences of hCol-01 to hCol-89 of Table K and L, or fragments thereof.

Herein, preferably, a PVTD is derived from a collagen or collagen-like protein. Being a prokaryotic or viral trimerisation domain, the PVTD is preferably derived from prokaryotic or viral collagen or collagen-like proteins, and more preferably from a viral or bacterial sequence present within a prokaryotic cell genome, preferably a bacterial cell genome, preferably a gram negative bacterial cell genome, preferably an E. coli genome, and most preferably from a O157:H7 E. coli strain. Preferably, the sequence is phage derived. It is envisaged that PVTDs from non-collagen proteins which naturally form trimers and/or triple helices may also be suitable for use in the present invention. Examples of PVTDs from non-collagen proteins are PfN domains from side tail fibre proteins in phages and E. coli genomes, “Collar” domains and “phage tail fibre” repeats domains in tail fiber family proteins, C-terminal domains from trimeric fibritin molecules, or other similar proteins or molecules known to persons skilled in the art.

Reference herein to “a” PVTD within a fusion protein or polypeptide includes either a single PVTD or a plurality of PVTD's. Thus, a fusion protein or polypeptide of the invention may comprise one, two, three, four, five, six, seven, eight, nine or ten or more independently selected PVTD's.

Reference herein to a PVTD includes both the monomeric form, and a dimeric or trimeric form.

The PVTD may be provided within the eukaryotic collagen or collagen-like domain, and/or at one or both ends thereof. A PVTD provided at the end of a eukaryotic domain may serve as a capping domain.

Preferred PVTD domains of the present invention may be independently selected from

i) the group consisting of any one of EPcIA-001 to EPcIA-142 of Table A, EPcIB-001 to EPcIB-021 of Table B, EPcIC-001 to EPcIC-005 of Table C, or EPcID-001 of Table D, PfN-01 to PfN-86 of Table H, PCoil-01 to PCoil-46 of Table I, PfC-01 to PfC-61 of Table J, and a Pf2 sequence, preferably one of the Pf2 domains in sequences EPcIB-001 to EPcIB-021 of Table B, or fragments or derivatives thereof; or an amino acid sequence having at least 50%, 60%, 70%, 80%, 90%, 91%, 92%, 93%, 94%, 95%, 96%, 97%, 98% or 99% sequence identity therewith;
ii) a PVTD encoded by a nucleic acid sequence selected from a nucleic acid sequence of Table E to G and M to R, or a derivative or fragment thereof;
iii) a PVTD encoded by a nucleic acid sequence having at least 50%, 60%, 70%, 80%, 90%, 91%, 92%, 93%, 94%, 95%, 96%, 97%, 98% or 99% sequence identity with a nucleic acid sequence of H);
iv) a PVTD encoded by a fragment of a nucleic acid sequence of i) to iii).

A PVTD may be identified and isolated from a longer sequence provided herein by a person skilled in the art. PVTD sequences are recognisable by having a non-collagen like sequence and by their three dimensional structure. Suitable PVTD's can be determined by their ability to hold collagen or collagen-like sequences in a trimer and preferably triple helical structure, and preferably to mediate one or more of the above mentioned functional characteristics of improved solubility, stability, thermal reversibility and lack of degradation. Preferred PVTD's are the PfN, PfC, Pf2 and PCoil sequences disclosed herein.

It is envisaged that any of the PVTD's disclosed herein may serve to provide increased thermal stability, increased solubility, improved resistance of fusion polypeptides to degradation, and/or improved reforming after denaturation. Preferably, however, one or more PfC domains may be used to provide thermal stability of a fusion protein and/or thermal reversibility; and one or more PfN and/or PCoil domains may be used to provide improved solubility as defined herein. Preferably, one or more PfC, PfN and/or PCoil sequences are used as capping domains, flanking one or both ends of a eukaryotic collagen or collagen-like domain. More preferably, PCoil sequences are provided within the fusion protein or polypeptide and not flanking an end thereof.

In the present invention, in a variant or derivative, the substitutions may be conservative substitutions, in which the amino acids or nucleic acids are replaced by amino acids or nucleic acids having similar properties such that the nature and activity of the sequence is not changed. Alternatively, the substitutions may be non-conservative, such that they are replaced by those having different properties which in turn affect the nature and properties of the sequence. Derivatives also include those sequences where one or more amino acids or nucleic acids have been added or deleted. Variants and derivatives also include combinations which have been engineered for a particular purpose and are not seen in nature. The monomers of such variants or derivatives may be naturally occurring or variant. Specific biological effects can be elicited by treatment with a derivative or fragment of limited function. For example, use of a derivative of collagen in a product or in treatment may have preferred biological activity or fewer side effects in a subject relative to treatment with the naturally occurring form of the collagen protein variants or derivatives or fragments of prokaryotic or viral sequences may affect the formation, structure or activity of a fusion protein or polypeptide of the invention.

“Sequence identity” is expressed as a percentage. The measurement of sequence identity of a nucleotide sequences is a method well known to those skilled in the art, using computer implementated mathematical algorithms such as ALIGN (Version 2.0), GAP, BESTFIT, BLAST (Altschul et al J. Mol. Biol. 215: 403 (1990)), FASTA and TFASTA (Wisconsin Genetic Software Package Version 8, available from Genetics Computer Group, Accelrys Inc. San Diego, Calif.), and CLUSTAL (Higgins et al, Gene 73: 237-244 (1998)), using default parameters.

Nucleic acid molecules defined herein as having sequence identity with a reference sequence may alternatively be defined as being capable of hybridising under stringent conditions to the complement of the reference sequence. Stringent hybridisation conditions are defined as those conditions under which a nucleotide sequence will preferentially hybridize to a target sequence. Increasing the stringency of the hybridisation conditions enables sequences of higher sequence identity to be found. Typical hybridisation conditions are 30-60° C., pH 7.0 to 8.3 and a salt concentration of less than 1.5 M Na⁺ ions. Preferred stringent hybridisation conditions hybridisation in 1M NaCl, 1% SDS at 37° C., and 50% formamide and washing in 0.1×SSC at 60 to 65° C.

“Naturally occurring,” as used with reference to the present invention refers to the fact that the object can be found in nature, for example is present in an organism, including viruses, and can be isolated from a source in nature and has not been intentionally modified by humankind in the laboratory. For example, a “naturally occurring” protein or polypeptide is one which exists in the same state as it exists in nature; i.e., it is not isolated, purified, recombinant, or cloned.

“Isolated” or “purified”, as used with reference to the present invention refers to an object which is substantially free of cellular material or other contaminating proteins from the cell or tissue source from which it is derived, for example enzymes, reagents, non-collagenous materials, telopeptides, prions, viruses, glycoproteins, lipids, and/or telopeptides that may cause disease, inflammatory and/or immunological reactions or substantially free from chemical precursors or other chemicals when chemically synthesized. The language “substantially free of cellular material” includes preparations in which the object is separated from cellular components of the cells from which it is isolated or recombinantly produced. Thus, it may comprise less than about 30%, 20%, 10%, or 5% (by dry weight) of any “contaminating” material. When a protein or polypeptide is recombinantly produced, it is also preferably substantially free of culture medium, i.e., culture medium represents less than about 20%, 10%, or 5% of the volume of the protein preparation. When a protein or polypeptide is produced by chemical synthesis, it is preferably substantially free of chemical precursors or other chemicals, i.e., it is separated from chemical precursors or other chemicals which are involved in the synthesis of the protein. Accordingly such preparations have less than about 30%, 20%, 10%, 5% (by dry weight) of chemical precursors or non-collagen chemicals.

Any protein or polypeptides used in the present invention, including the collagen, collagen-like and PVTD sequences, may be modified to alter stability, functionality or physiochemical properties. Such modification includes addition of one or more polyethylene glycol molecules, sugars, phosphates, and/or other such molecules, where the molecule or molecules are not naturally attached to the corresponding wild-type polypeptides or proteins. Suitable chemical modifications and methods modifying by chemical synthesis are well known to those of skill in the art. The same type of modification may be present in the same or varying degree at several sites on the protein. Furthermore, modifications can occur anywhere in the sequence, including on the backbone, on any amino acid side-chains and at the amino or carboxyl termini. Accordingly, a given polypeptide or protein may contain one or more of the same or different types of modifications.

Such variants, derivatives or modified polypeptides or proteins may be structurally substantially similar in both three-dimensional shape and biological activity to a naturally occurring polypeptide or protein and may preferably comprise a spatial arrangement of reactive chemical moieties that closely resembles the three-dimensional arrangement of active groups in the naturally occurring polypeptide or protein. Further modifications can be made by replacing chemical groups of the amino acids with other chemical groups of similar structure. These modifications include incorporating amino acids which are not directly encoded by the universal genetic code, or non-natural amino acids. Amino acids may be incorporated into the polypeptide chain using alternative peptide bond linkages (for example R-amino acids).

Additionally, a polypeptide or protein used in the present invention, for example the collagen or collagen-like protein or polypeptide or PVTD, may be structurally modified to comprise one or more D-amino acids. For example, the polypeptide or protein may be an enantiomer in which one or more L-amino acid residues in the amino acid sequence is replaced with the corresponding D-amino acid residue or a reverse-D polypeptide, which is a polypeptide consisting of D-amino acids arranged in a reverse order as compared to the L-amino acid sequence described above (Smith et al. (1988), Drug Develop. Res. 15:371-379). Methods of producing suitable structurally modified polypeptides are well known in the art

Suitable derivatives may be identified by screening combinatorial libraries of mutants, e.g., truncation mutants. Libraries of mutants may be generated using techniques such as combinatorial mutagenesis, enzymatically ligating a mixture of synthetic oligonucleotides into gene sequences such that a degenerate set of potential polypeptide or protein sequences is expressible as individual polypeptides, or alternatively, as a set of larger fusion proteins (e.g., for phage display). There are a variety of methods which can be used to produce libraries of potential collagen derivatives from a degenerate oligonucleotide sequence. Chemical synthesis of a degenerate gene sequence can be performed in an automatic DNA synthesiser, and the synthetic gene then ligated into an appropriate expression vector. Use of a degenerate set of genes allows for the provision, in one mixture, of all of the sequences encoding the desired set of potential sequences. Methods for synthesizing degenerate oligonucleotides are known in the art (see, e.g., Narang (1983), Tetrahedron 39:3-22; Itakura et al. (1984), Ann. Rev. Biochem. 53:323-356; Itakura et al. (1977), Science 198:1056-1063; Ike et al. (1983), Nucleic Acids Res. 11:477-488).

By “operably linked” means that domains and/or sequences within a fusion polypeptide or protein are linked in a manner which allows some or all of the biological activity of one or more of the sequences to be retained. The same definition is used herein with reference to the nucleic acid sequences and expression vectors of the invention. As an example, in relation to polypeptide sequences, where two or more are operably linked, each may retain some or all of its biological activity. Where two or more nucleic acid sequences are operably linked, this may mean that they are positioned in relation to each other such that one may direct transcription of the other, in the presence of any necessary molecules such as transcription factors.

The present invention also provides a nucleic acid sequence encoding a fusion protein or polypeptide of the invention. Typically, the nucleic acid sequence will encode a eukaryotic collagen or collagen-like domain comprising, or flanked at one or both ends, by one or more PVTDs, as previously described herein.

The fusion polypeptides of the fusion protein may be encoded by a single nucleic acid sequence or a plurality (two, three, four, five, six, seven, eight, nine, or ten or more) nucleic acid sequences. A plurality of nucleic acid sequences may be operably linked. The fusion protein may be encoded by a single nucleic acid sequence or two or more nucleic acid sequences, which may or may not be operably linked.

Nucleic acid sequences encoding the PVTDs as described herein include:

i) a nucleic acid sequence which encodes an amino acid sequence of any one of EPcIA-001 to EPcIA-142 of Table A, EPcIB-001 to EPcIB-021 of Table B, EPcIC-001 to EPcIC-005 of Table C, or EPcID-001 of Table D, PfN-01 to PfN-86 of Table H, PCoil-01 to PCoil-46 of Table I, PfC-01 to PfC-61 of Table J, and a Pf2 sequence, preferably one of the Pf2 domains in sequences EPcIB-001 to EPcIB-021 of Table B; or a nucleic acid sequence encoding an amino acid sequence having at least 50%, 60%, 70%, 80%, 90%, 91%, 92%, 93%, 94%, 95%, 96%, 97%, 98% or 99% sequence identity therewith;
ii) a nucleic acid sequence selected from a nucleic acid sequence of Table E to G and M to R, or a nucleic acid sequence having at least 50%, 60%, 70%, 80%, 90%, 91%, 92%, 93%, 94%, 95%, 96%, 97%, 98% or 99% sequence identity therewith;
iii) a fragment or derivative of a nucleic acid sequence of i) to iii) which encodes a polypeptide which functions as a PVTD.

Nucleic acid sequences encoding the eukaryotic collagen or collagen like domains as described herein include:

i) a nucleic acid sequence which encodes an amino acid sequence of any one of hCol01-089 of Table K and L; or a nucleic acid sequence which encodes an amino acid sequence having at least 50%, 60%, 70%, 80%, 90%, 91%, 92%, 93%, 94%, 95%, 96%, 97%, 98% or 99% sequence identity therewith;
ii) a nucleic acid sequence selected from a nucleic acid sequence of Table S to V, or a nucleic acid sequence having at least 50%, 60%, 70%, 80%, 90%, 91%, 92%, 93%, 94%, 95%, 96%, 97%, 98% or 99% sequence identity therewith;
iii) a fragment or derivative of a nucleic acid sequence of i) to iii), which encodes a collagen or collagen-like domain.

Preferably, the eukaryotic and prokaryotic domains and sequences of a fusion polypeptide or protein will be encoded as a contiguous sequence, such that they are operably linked.

Each trimeric fusion protein of the invention will be the result of trimerisation of three monomer fusion proteins of the invention, which can be identical or different and therefore encoded by the same or different nucleic acid sequences. Preferably, where two or more nucleic acid sequences encoding fusion polypeptides are provided, they are such that when expressed together they are able to cooperate (with one or more other fusion polypeptides) to form a triple helix. Preferably, PVTDs that flank one or both ends of the collagen or collagen-like domains are selected such that they are able to cooperate with PVTDs of other monomers to form trimers, and thus mediate the formation of collagen triple helices.

Nucleic acid sequences encoding sequences described herein may be obtained by screening cDNA libraries (e.g., libraries generated by recombining homologous nucleic acids as in typical recursive recombination methods) using oligonucleotide probes which can hybridize to, or PCR-amplify, polynucleotides which encode known sequences or preferred motifs. Procedures for screening and isolating cDNA clones are well-known to those of skill in the art. Such techniques are described in, for example, Molecular cloning: a laboratory manual, 3rd edition (2001), by J. Sambrook & D. Russell, Cold Spring Harbor Laboratory Press, Cold Spring Harbor, N.Y. (“Sambrook & Russell”), and Current Protocols in Molecular Biology (2010, regularly supplemented since 1987, last update Jan. 25, 2010), F. M. Ausubel et al. editors, Wiley Interscience (“Ausubel”). Alternatively, nucleic acid sequences including designed sequences not found in nature can be synthesized by conventional techniques including automated DNA synthesizers. Synthesis of genes of almost any length is available commercially from several providers and is a well-known technique to those of skill in the art.

To provide the eukaryotic collagen polypeptides with the appropriate signal and secretion peptides, a nucleic acid sequence encoding a polypeptide may additionally comprise nucleic acid sequences encoding signal and/or secretion peptides, in addition to any further sequences which are required for post-translational processing or transport of the fusion protein or polypeptide. Preferably, nucleic acid sequences encoding the peptides will be operably linked to the nucleic acid sequence encoding the fusion protein or polypeptide. Preferably, the nucleic acid sequences will be provided as a contiguous sequence encoding a fusion protein or polypeptide and signal and/or secretion peptides as a single polypeptide sequence.

Variant nucleic acid sequences can be created by introducing one or more nucleotide substitutions, additions or deletions into the naturally occurring nucleotide sequence such that one or more amino acid substitutions, additions or deletions are introduced into the encoded protein. Mutations can be introduced by standard techniques, such as site-directed mutagenesis and PCR-mediated mutagenesis and nucleic acid synthesis. Preferably, conservative amino acid substitutions are made at one or more predicted non-essential amino acid residues. Thus, for example, 1%, 2%, 3%, 5%, or 10% of the amino acids can be replaced by conservative substitution. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue is preferably replaced with another amino acid residue from the same side chain family. Alternatively, mutations can be introduced randomly along all or part of a collagen coding sequence, such as by saturation mutagenesis, and the resultant mutants can be screened for biological activity to identify mutants that retain activity. Following mutagenesis, the encoded protein can be expressed recombinantly and the activity of the protein can be determined.

Preferably, a nucleic acid sequence of the fifth aspect of the invention protein is produced by standard recombination DNA techniques. For example, DNA sequences coding for the different domains are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the nucleic acid sequence of the invention may be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see for example Current Protocols in Molecular Biology (2010, regularly supplemented since 1987, last update Jan. 25, 2010), F. M. Ausubel et al. editors, Wiley Interscience).

In embodiments, nucleic acid sequences of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids ((see Hyrup & Nielsen (1996), Bioorg. Med. Chem. 4:5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) supra; Perry-O'Keefe et al. (1996), Proc. Natl. Acad. Sci. USA 93:14670-675.

In the present invention, a “recombinant nucleic acid” (e.g., DNA or RNA) molecule or sequence means, for example, a nucleic acid sequence that is not naturally occurring or is made by the combination (for example, artificial combination) of at least two segments of sequence that are not typically included together, not typically associated with one another, or are otherwise typically separated from one another. A recombinant nucleic acid sequence can comprise a nucleic acid molecule formed by the joining together or combination of nucleic acid segments from different sources and/or artificially synthesized. The term “recombinantly produced” refers to an artificial combination usually accomplished by either chemical synthesis means, recursive sequence recombination of nucleic acid segments or other diversity generation methods of nucleotides, or manipulation of isolated segments of nucleic acids, e.g., by genetic engineering techniques known to those of ordinary skill in the art. “Recombinantly expressed” typically refers to techniques for the production of a recombinant nucleic acid in vitro and transfer of the recombinant nucleic acid into cells in vivo, in vitro, or ex vivo where it may be expressed or propagated. A “recombinant polypeptide” or “recombinant protein” usually refers to polypeptide or protein, respectively, that results from a cloned or recombinant gene or nucleic acid.

A nucleic acid sequence or polypeptide is “recombinant” when it is artificial or engineered, or derived from an artificial or engineered protein or nucleic acid. The term “recombinant” when used with reference e.g., to a cell, nucleic acid sequence, expression vector, or polypeptide typically indicates that the cell, nucleic acid sequence, or expression vector has been modified by the introduction of a heterologous (or foreign) nucleic acid or the alteration of a native nucleic acid, or that the polypeptide has been modified by the introduction of a heterologous amino acid, or that the cell is derived from a cell so modified. Recombinant cells express nucleic acid sequences (e.g., genes) that are not found in the native (non-recombinant) form of the cell or express native nucleic acid sequences (e.g., genes) that would be abnormally expressed, under-expressed, or not expressed at acid.

The present invention also provides a vector comprising a nucleic acid sequence of the invention. Preferably, the vector will comprise one, two or three nucleic acid sequences of the invention, which when expressed may cooperate to form a trimeric, preferably a triple-helical, protein where the triple helical domains form a correct collagen or collagen-like helix. Preferably, the vector is an expression vector. Alternatively, it is envisaged that a plurality of vectors may be used to express a fusion polypeptide or fusion protein of the invention. In this embodiment, two, three, four, five, or six or more vectors may be used, each encoding all or part of a fusion polypeptide or fusion protein, which when expressed operably cooperate to form a polypeptide chain, fusion polypeptide or fusion protein of the invention.

A vector is a composition for facilitating cell transduction by a selected nucleic acid, or expression of the nucleic acid in the cell. Vectors include, e.g., plasmids, cosmids, viruses, YACs, BACs, bacteria, poly-lysine, etc. An “expression vector” is a nucleic acid construct, generated recombinantly or synthetically, with a series of specific nucleic acid elements that permit transcription of a particular nucleic acid sequence in a host cell. The vector can be part of a plasmid, virus, or nucleic acid fragment. In a preferred aspect of this embodiment, the construct further comprises regulatory sequences, including, for example, a promoter, operably linked to the sequence. Large numbers of suitable vectors and promoters are known to those of skill in the art, and are commercially available.

General texts which describe molecular biological techniques useful herein, including the use of vectors, promoters and many other relevant topics, include Guide to Molecular Cloning Techniques, Methods in Enzymology, 152 (1987), S. L. Berger & A. R. Kimmel eds, Academic Press, San Diego, Calif. (“Berger & Kimmel”); Sambrook & Russell, supra, and Ausubel, supra.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, expression vectors, are capable of directing the expression of genes to which they are operatively linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The vectors of the invention may comprise a nucleic acid sequence of the invention in a form suitable for expression of the nucleic acid in a host cell, which means that the vectors include one or more regulatory sequences, selected on the basis of the host cells to be used for expression, which is operatively linked to the nucleic acid sequence to be expressed. Within a vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Gene Expression Technology, Methods in Enzymology, 185 (1990), D. V. Goeddel, editor, Academic Press, San Diego, Calif. Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the vector can depend on such factors as the choice of the host cell to be transformed, the level of expression of protein desired, etc. The vectors of the invention can be introduced into host cells to thereby produce proteins or polypeptides, including fusion proteins or polypeptides, encoded by nucleic acids as described herein.

The vectors of the invention can be designed for expression of the fusion protein or polypeptide of the invention in prokaryotic or eukaryotic cells, preferably the former. Most preferably, the fusion protein or polypeptide is expressed in bacterial cells, and most preferably the same species of cells from which the prokaryotic collagen trimerisation domains are derived from e.g., bacterial cells such as E. coli. Alternatively the fusion protein may be expressed in other host cell types such as yeast, insect, mammalian, fish or plant. The vector may be designed for in vitro or ex vivo expression.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin, TEV protease and enterokinase. Typical fusion expression vectors include pGEX (Pharmacia Biotech Inc; Smith & Johnson (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al. (1988) Gene 69:301-315) and pET 11d (Studier et al. (1990), in Gene Expression Technology, Methods in Enzymology 185, D. V. Goeddel, ed, Academic Press, San Diego, Calif., pp. 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV5 promoter.

One strategy to maximize recombinant protein expression in E. coli is to express the protein in a bacterial strain having an impaired capacity to proteolytically cleave the recombinant protein (Gottesman, Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In a further aspect, the present invention provides a host cell comprising any one or more of the above described fusion protein, nucleic acid sequence or vector. The host cell can be a eukaryotic cell, such as a plant cell, an insect cell, a mammalian cell (such as Chinese hamster ovary cells (CHO) or COS cells), a yeast cell, or the host cell can be a prokaryotic cell, such as a bacterial cell (e.g., an E. coli cell). Most preferably, the host cell will be a bacterial cell. Preferably, the host cell will be of the same species as that from which the prokaryotic collagen trimerisation domains are derived, examples of which include E. coli, Streptococcus and Bacillus. Suitable host cells will be known to persons skilled in the art.

Different host cells have specific cellular machinery and characteristic mechanisms for such post-translational activities and can be chosen to ensure the correct modification and processing of the introduced protein.

The terms “host cell” and “recombinant host cell” are used interchangeably herein. Such terms refer not only to the particular subject cell, but also to the progeny or potential progeny of such a cell. Because certain modifications may occur in succeeding generations due to either mutation or environmental influences, such progeny may not, in fact, be identical to the parent cell, but are still included within the scope of the term as used herein.

For long-term, high-yield production of the fusion proteins or polypeptides, cell lines may be established, which stably express a fusion protein of the invention. The cells are transduced using the vectors of the invention, which contain viral origins of replication or endogenous expression elements and a selectable marker gene. Following the introduction of the vector into the cells, they are allowed to grow for 1-2 days in an enriched media before they are switched to selective media. The purpose of the selectable marker is to confer resistance to selection, and its presence allows growth and recovery of cells which successfully express the introduced sequences. For example, resistant clumps of stably transformed cells can be proliferated using tissue culture techniques appropriate to the cell type.

For stable transfection of mammalian cells, it is known that, depending upon the vector and transfection technique used, only a small fraction of cells may integrate the foreign DNA into their genome. In some cases vector DNA is retained by the host cell. In other cases the host cell does not retain vector DNA and retains only an isolated nucleic acid molecule of the invention carried by the vector. In some cases, and isolated nucleic acid sequence of the invention is used to transform a cell without the use of a vector.

Preferred selectable markers include those which confer resistance to drugs, such as G418, hygromycin and methotrexate. Nucleic acid encoding a selectable marker can be introduced into a host cell on the same vector as the nucleic acid encoding the fusion protein, or can be introduced on a separate vector. Cells stably transfected with the introduced nucleic acid can be identified by drug selection (e.g., cells that have incorporated the selectable marker gene will survive, while the other cells die).

The present invention also provides an extract from a host cell, which comprises any one or more of the fusion polypeptide or protein, nucleic acid sequence and/or vector of the invention. The extract may be a cellular lysate.

The fusion proteins, polypeptides, nucleic acid sequences, vectors and/or host cells of the invention can also be used to produce non-human transgenic animals. The fusion proteins of the invention, and the nucleic acid sequences coding for fusion proteins of the invention, can also be used to produce non-human transgenic animals through application of the appropriate technology. Thus, the present invention provides a non-human, insect or animal comprising a host cell of the invention.

A host cell of the invention, such as a prokaryotic or eukaryotic host cell in culture, can be used to produce (i.e., express) a fusion protein or polypeptide of the invention. Accordingly, the invention further provides a method of producing a fusion protein or polypeptide comprising a eukaryotic collagen or collagen-like domain and one or more PVTDs, the method comprising:

i) introducing into a host cell one or more nucleic acid sequences encoding a eukaryotic collagen or collagen-like domain comprising, or flanked by, one or more PVTDs;
ii) culturing the host cell under conditions suitable for expression and formation of the fusion polypeptide or protein in the host cell, and preferably the formation of a trimeric assembly of the fusion protein; and
iii) isolating the expressed fusion protein or polypeptide from the host cell.

Preferably, the nucleic acid sequence is that of the fifth aspect. The nucleic acid sequence may be provided in the host cell as a vector of the fourth aspect.

Introduction of the construct into the host cell can be effected by calcium phosphate transfection, DEAE-Dextran mediated transfection, electroporation, or other common techniques (Davis, L., Dibner, M., and Battey, I. (1986) Basic Methods in Molecular Biology, Sambrook and Ausubel, supra.).

Host cells transformed with a nucleic acid sequence of the invention are optionally cultured under conditions suitable for the expression and recovery of the encoded protein from cell culture. The fusion protein or polypeptide produced by a recombinant cell can be secreted, membrane-bound, or contained intracellularly, depending on the sequence and/or the vector used. As will be understood by those of skill in the art, vectors containing nucleic acid sequences encoding fusion proteins or polypeptide of the invention can be designed with signal sequences which direct secretion of the polypeptides through a prokaryotic or eukaryotic cell membrane.

The engineered host cells can be cultured in conventional nutrient media modified as appropriate for activating promoters, selecting transformants, or amplifying the nucleic acid sequences and/or expression vector. The culture conditions, such as temperature, pH and the like, will be apparent to those skilled in the art. In addition to Sambrook & Russell, Berger & Kimmel and Ausubel, details regarding cell culture can be found in Payne et al. (1992) Plant Cell and Tissue Culture in Liquid Systems, John Wiley & Sons, New York, N.Y.; Gamborg and Phillips (eds.) (1995) Plant Cell, Tissue and Organ Culture, Fundamental Methods Springer Lab Manual, Springer-Verlag (Berlin Heidelberg, N.Y.); and Atlas and Parks (eds.) The Handbook of Microbiological Media (1993) CRC Press, Boca Raton, Fla.

Cell-free transcription/translation systems can also be employed to produce the fusion proteins or polypeptides, using the nucleic acid sequences and/or expression vectors of the present invention. Methods will be known to persons skilled in the art, and are detailed in Tymms (1995) In vitro Transcription and Translation Protocols: Methods in Molecular Biology Volume 37, Garland Publishing, NY.

Following transduction of a suitable host cell line or strain and growth of the host strain to an appropriate cell density, the selected promoter is induced by appropriate means (e.g., temperature shift or chemical induction) and cells are cultured for an additional period. The fusion protein is then recovered from the culture medium. Alternatively, cells can be harvested by centrifugation, disrupted by physical or chemical means, and the resulting crude extract retained for further purification. Eukaryotic or prokaryotic cells employed in expression of proteins can be disrupted by any convenient method, including freeze-thaw cycling, sonication, mechanical disruption, or by the use of cell lysing agents, or other methods, which are well know to those skilled in the art.

Preferably, the method may further comprise downstream processing of the fusion polypeptide or protein.

The nucleic acid sequences of the present invention may be operably linked to a marker sequence which facilitates purification of the encoded protein. Such purification facilitating domains include, but are not limited to, metal chelating peptides such as poly-histidine modules that allow purification on immobilized metals, a sequence which binds glutathione (e.g., GST), a hemagglutinin (HA) tag (corresponding to an epitope derived from the influenza hemagglutinin protein (Wilson et al. (1984) Cell 37:767-778), maltose binding protein sequences, and/or the FLAG epitope utilized in the FLAGS extension/affinity purification system (Immunex Corp, Seattle, Wash.). The inclusion of a protease-cleavable polypeptide linker sequence between the purification domain and the nucleic acid sequence of the invention is useful to facilitate purification. In a preferred embodiment the fusion polypeptide or protein will be expressed using a vector containing a poly-histidine tag at the N-terminus, or at the C-terminus, or both, to facilitate purification using immobilized metal affinity chromatography. In another preferred embodiment the fusion polypeptide or protein will be expressed using a vector containing a poly-histidine tag at the N-terminus, or at the C-terminus, or both, in addition to one or more solubility enhancer domains in frame to the fusion protein to facilitate its soluble expression in bacterial expression systems. Examples of suitable solubility enhancer domains include but are not limited to GST, maltose binding protein (MBP) (Sachdev & Chirgwin (2000), Methods Enzymol. 326:312-321), N utilization substance A (NusA) (Nallamsetty & Waugh (2006), Protein Expr. Purif. 45:175-182, domain I of IF2 (Sarensen et al. (2003) Protein Expr. Purif. 32:252-259) or thioredoxin (Trx) (Sachdev & Chirgwin (1998) Protein Expr. Purif. 12:122-132).

In some aspects, it may be desirable to denature the expressed and purified fusion protein to provide a gelatine-like protein. A gelatine-like protein of the invention includes denatured collagen or collagen like proteins or collagen or collagen like fragments or mixtures thereof. Thus, a gelatine made in the present invention may comprise monomers or dimers of the fusion protein optionally in combination with fragments of the fusion protein or fusion polypeptide. In the context of the present invention, any degree of denaturing is envisaged, which may be complete or partial loss of the tertiary structure of the fusion protein, and/or complete or partial uncoiling of the triple helix.

The denaturing may be the eukaryotic portion of the fusion protein, or may additionally comprise denaturing of the one or more PVTDs present.

Gelatines from animal origin are denatured forms of type I collagens from animal skins, bones and hides. Thus, it contains polypeptide sequences having Gly-X-Y repeats, where X and Y are most often proline and hydroxyproline residues. These sequences contribute to triple helical structure and affect the gelling ability of gelatine polypeptides. However, it is also possible to manufacture unhydroxylated gelatine from collagens produced in the absence of prolyl hydroxylation (see for example U.S. Pat. No. 6,413,742).

Collagen can be denatured to produce gelatin utilizing detergents, heat or denaturing agents. Additionally, these methods, processes, and techniques include, but are not limited to, treatments with strong alkali or strong acids, heat extraction in aqueous solution, ion exchange chromatography, cross-flow filtration and heat drying, and other methods that may be applied to collagen to produce the gelatine.

The expressed protein can be recovered and purified from recombinant cell cultures by any of a number of methods well known in the art, including ammonium sulfate or ethanol precipitation, acid extraction, anion or cation exchange chromatography, size exclusion chromatography, hydrophobic interaction chromatography, affinity chromatography (e.g., using any of the tagging systems noted herein), hydroxyapatite chromatography, and lectin chromatography. Protein refolding steps can be used, as desired, in completing configuration of the mature protein. Fast protein liquid chromatography (FPLC) and High performance liquid chromatography (HPLC) can be employed if appropriate in any of the purification steps.

A nucleic acid, polypeptide, or other component is substantially pure when it is partially or completely recovered or separated from other components of its natural environment such that it is the predominant species present in a composition, mixture, or collection of components (i.e., on a molar basis it is more abundant than any other individual species in the composition). In preferred embodiments, the preparation consists of more than 70%, typically more than 80%, or preferably more than 90% of the isolated species.

In an eighth aspect of the invention, there is provided a product comprising any one or more of a fusion polypeptide or protein, nucleic acid sequence, expression vector and/or host cell of the invention. Products include compositions, foodstuffs, cosmetic, medicament, artificial tissue, pharmaceutical, dietary supplement, reagent and glue.

Where the product is a composition, this may be made by admixing any one or more of the fusion proteins, nucleic acid sequences, expression vectors and/or host cells of the present invention with one or more optional excipients and other optional ingredients. Examples of suitable excipients include, but are not limited to any of the vehicles, carriers, buffers and stabilizers that are well known in the art.

Where the composition is a pharmaceutical composition, the composition may contain, in addition to any one or more of the fusion polypeptides, proteins, nucleic acid sequences, expression vectors and/or host cells of the present invention, one or more further pharmaceutically active agents, wherein the resulting combination composition may be further admixed with an excipient. Pharmaceutically acceptable excipients are well known in the art, and disclosed in, for example, Handbook of Pharmaceutical Excipients, (Fifth Edition, October 2005, Pharmaceutical Press, Eds. Rowe R C, Sheskey P J and Weller P). “Pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Suitable further pharmaceutically active agents include, but are not limited to, hemostatics (such as thrombin, fibrinogen, ADP, ATP, calcium, magnesium, TXA2, serotonin, epinephrine, platelet factor 4, factor V, factor XI, PAI-1, thrombospondin and the like and combinations thereof), anti-infectives (such as antibodies, antigens, antibiotics, antiviral agents and the like and combinations thereof), analgesics and analgesic combinations or, anti-inflammatory agents (such as antihistamines).

Preferably, the composition may additionally comprise a surfactant (or with another component of a cleaning solution such as a builder, a polymer, a bleach system, a structurant, a pH adjuster, a humectant, or a neutral inorganic salt) and/or an excipient (optionally a pharmaceutically acceptable excipient), such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

The active ingredients of the composition, for example any one or more of the fusion polypeptides or proteins, nucleic acid sequences, expression vectors and/or host cells of the present invention and any secondary pharmaceutically active agent are preferably present in the composition in an effective amount. An “effective amount” means a dosage or amount sufficient to produce a desired result. The desired result may comprise an objective or subjective improvement in the recipient which receives the dosage or amount.

A composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as thylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

Such a pharmaceutical composition may be used for various purposes, including but not limited to diagnostic, therapeutic and/or preventative purposes.

The composition may be provided in a kit, e.g. sealed in a suitable container that protects the contents from the external environment. Such a kit may include instructions for use. The kit may additionally comprise other compositions, which may be administered substantially simultaneously or sequentially with a pharmaceutical composition of the present invention.

In an eleventh aspect of the invention, there is provided the use of any one or more of a fusion polypeptide or protein, nucleic acid sequence, vector, gelatine-like protein or host cell of the invention in the treatment or prevention of a condition selected from the group consisting of osteoarthritis, dystrophic epidermolysis bullosa, urinary incontinence disorders, dental and skeletal injuries, in the treatment and healing of wounds and burns, in the manufacture of haemostatic sponges and sutures used by surgeons, in cartilage regeneration, in vascular graft coatings, and in several plastic surgery applications (tissue augmentation, implants and dermal fillings).

The composition may be administered alone or in combination with other treatments, either substantially simultaneously or sequentially dependent upon the condition to be treated.

Any one or more of the fusion polypeptide, protein, nucleic acid sequence, vector, gelatine-like protein or host cells of the invention may be useful in the treatment or prevention of connective tissue malfunction or damage, wherein the subject is administered one of the above mentioned products of the invention in an amount effect to treat the condition/disease/disorder, including wherein the subject is a mammal (e.g., a human), and wherein the product of the invention is administered in vivo, in vitro, or ex vivo (or a combination of such) to one or more cells of the subject. An effective amount is as defined above. Conditions which may benefit from treatment with collagen based products of the invention include plastic surgery, dermatology, and/or amputee stump revision, osteogenesis imperfecta, Ehlers-Danlos Syndrome, Infantaile cortical hyperostosis, collagenopathy (types II and XI), Alport syndrome, Goodpastures syndrome, Ulrich myopathy, Bethlem myopathy, epidermolysis bullosa dystrophica, posterior polymorphous corneal dystrophy 2, EDM2 and EDM3, schmid metaphyseal dysplasia, bullus pemphigoid and junctional epidermylosis bullosaa, and atopic dermatitis.

Treatment may be administered to a subject who displays symptoms or signs of pathology, disease, or disorder, in which treatment is administered to such subject for the purpose of diminishing or eliminating those signs or symptoms of pathology, disease, or disorder. The therapeutic activity of the products of the invention may eliminate or diminish signs or symptoms of pathology, disease or disorder, when administered to a subject suffering from such signs or symptoms.

In a further aspect of the invention, there is provided a collagen-based product, for example a foodstuff, cosmetic, medical device, medicament, artificial tissue, scaffold, pharmaceutical, dietary supplement, chemical or biochemical reagent or glue, comprising any one or more of fusion polypeptide, protein, nucleic acid sequence, vector, gelatin-like protein or host cell according to the invention.

In a tenth aspect of the invention, there is provided the use of any one or more of a fusion polypeptide, protein, nucleic acid sequence, vector, gelatin-like protein or host cell of the invention, in a collagen-based product, for example a foodstuff, cosmetic, medical device, medicament, artificial tissue, scaffold, pharmaceutical, dietary supplement, chemical or biochemical reagent or glue.

Collagen-based products include any product which requires collagen, and is not limited to the products listed above.

A product of the invention may be a foodstuff, comprising any one or more of a fusion polypeptide, protein, nucleic acid sequence, vector, gelatin-like protein or host cell of the invention, or a denatured gelatin-like protein of the invention. In preferred embodiments, the foodstuff comprises any one or more of a fusion polypeptide, protein or a denatured gelatin-like protein of the invention. The foodstuff may additionally comprise flavourings, preservatives, colouring agents, thickening agents, gelling agents, and any other suitable additives for use in nutritional products. Examples of foodstuffs include emulsifying agents, foam stabilizer, or a thickening agent. Preferred foodstuffs include sweets, gelatin powder, protein drinks, energy bars, wine, beer, fruit juice, food colouring agents and dried food products. The foodstuff may be one which is suitable for human or animal consumption.

Collagen is widely used in cosmetics, and a product of in the present invention may be cosmetic which comprises any one or more of a fusion polypeptide, fusion protein, nucleic acid sequence, vector, host cell, or a denatured gelatine-like fusion protein of the invention. Preferably, the cosmetic will include a fusion protein of the invention, or a denatured gelatin-like protein or fusion polypeptide of the invention. The cosmetic may be in the form of a cream, powder, membrane, matrix, lotion, liquid, film, foam, sponge or mask, a composite of the two or more of these forms, or in any other form. Preferred cosmetics include hair products including shampoo, conditioner, injectable fillers and topical skin applications such as make-up and moisturizers.

A collagen-based product may be a medicament. This may be a composition, as hereinbefore described, or may be in the form of an injectable substance, a pill, capsule, tablet, liquid, cream, lotion, film, sponge, matrix, membrane, powder, or indeed any other suitable form. In such a medicament, collagen may be used as a carrier for an active ingredient. Thus, also provided is a collagen-based product consisting of any one or more of a fusion polypeptide, protein, nucleic acid sequence, expression vector of host cell, or denatured gelatin-like protein according to the invention in combination with other suitable chemicals in the form of a material, to produce for example a capsule to house a pharmaceutical. Alternatively, in the medicament, the collagen-based product may be the active ingredient, and will be present in an effective amount, as previously defined. Such medicaments will preferably comprise one or more excipients, optional additional ingredients, optional secondary pharmaceutical products, as well as other optional ingredients, for example as defined in relation to the compositions above.

Collagen is often used as a dietary or nutritional supplement. Therefore, the present invention provides a supplement comprising an effective amount of any one or more of a fusion polypeptide, protein, nucleic acid sequence, expression vector, host cell or denatured gelatin-like protein of the invention, and a nutritionally acceptable carrier.

Also provided are medical devices comprising any one or more of a fusion polypeptide, protein, nucleic acid or host cell of the invention, or a denatured gelatine-like protein of the invention. Medical devices include products such as films, matrixes, membranes, sponges, and mask, non-implantable substrates, implants, coatings, shields, threads, patches, tubes, plugs, scaffolds, injectable collagen, bandages, wound dressings, and collagen for in vitro applications. The medical device may comprise a composite of two or more of these product types, eg. film/sponge or film/sponge/film.

Such medical devices may be useful in hernia repair, spinal tension band, annular repair for the spine, and/or for repair, reconstruction, augmentation or replacement of a sphincter, meniscus, nucleus, rotator cuff, breast, bladder, and/or vaginal wall, corneal implants, scar revision, contracture revision, hypertrophic scar treatment, cosmetics, cosmetic surgery, wrinkle removal, general surgical settings, spinal, vascular, and/or neurosurgical settings, sports medicine surgical applications, plastic surgery, dermatology, and/or amputee stump revision, repair or correct congenital anomalies or acquired defects. Examples of such conditions are congenital anomalies such as hemifacial microsomia, malar and zygomatic hypoplasia, unilateral mammary hypoplasia, pectus excavatum, pectoralis agenesis (Poland's anomaly), and velopharyngeal incompetence secondary to cleft palate repair or submucous cleft palate (as a retropharyngeal implant); acquired defects (post traumatic, post surgical, or post infectious) such as depressed scars, subcutaneous atrophy (e.g., secondary to discoid lupis erythematosis), keratotic lesions, enopthalmos in the unucleated eye (also superior sulcus syndrome), acne pitting of the face, linear scleroderma with subcutaneous atrophy, saddle-nose deformity, Romberg's disease, and unilateral vocal cord paralysis; and cosmetic defects such as glabellar frown lines, deep nasolabial creases, circum-oral geographical wrinkles, sunken cheeks, and mammary hypoplasia, as well as any other conditions not mentioned herein.

In particular, injectable collagen may be useful in cell delivery, drug delivery and provision of clear collagens, dispersed collagens, micronized collagens (cryogenic grinding), and/or collagen product mixtures, e.g., collagen mixed with thrombin.

The medical device may further comprise analgesic, anti-inflammatory, antibiotic, and/or growth factors.

Because the collagen product retains a portion of its collagen constituents that remain at least partly bound to each other and retain a portion of native non-collagenous proteins, medical devices comprising the fusion polypeptide, or fusion protein of the invention may be non-immunogenic, compared to collagen implants derived from other sources (e.g., bovine-derived collagen).

Medical devices such as films and/or coatings may be useful, for example, in barrier dressings (e g, adhesion barriers and barriers to liquids), occlusions, structural supports, osteochondral retainers for cells/matrices (+/− analgesic), drug delivery devices, e g, collagen product coating combined with, and wraps for bone defects. In addition, catheters and stents may be coated In a further implementation, a plasticizer, bioactive, bioabsorbable, soluble, and/or biocompatible component may be combined with the collagen product or the gelatine.

In the collagen-based products described herein, a fusion polypeptide or protein of the invention may be coated onto a solid surface or insoluble support. The support may be in particulate or solid form, including for example a plate, a test tube, beads, a ball, a filter, fabric, polymer or a membrane. Methods for fixing a protein to solid surfaces or insoluble supports are known to those skilled in the art. The support may be a protein, for example a plasma protein or a tissue protein, such as an immunoglobulin or fibronectin. Alternatively, the support may be synthetic, for example a biocompatible, biodegradable polymer. Suitable polymers include polyethylene glycols, polyglycolides, polylactides polyorthoesters, polyanhydrides, polyphosphazenes, and polyurethanes. The inclusion of reactive groups in the fusion protein allows chemical coupling to inert carriers such that resulting product may be delivered to the desired site without entry into the bloodstream.

Another product of the invention is a tissue scaffold, comprising host cells of the invention. In a preferred embodiment, host cells of the invention may be seeded onto a scaffold to produce collagen, or collagen fragments, which may then be used in the treatment of skin and/or tissue related disorders.

Also provided is a product for technical use, for example in photographic or technical applications. Such a product may comprise a fusion polypeptide fusion, protein according to the invention in combination with silver halide emulsions.

The compositions, nutritional supplements, cosmetics, medical devices and food stuffs of the invention will preferably suitable be for pharmaceutical use in a subject, including an animal or human.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The readers attention is directed to all papers and documents which are filed concurrently with or previous to this specification in connection with this application and which are open to public inspection with this specification, and the contents of all such papers and documents are incorporated herein by reference.

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth, the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

It is noted that, as used in this specification and the appended claims, the singular forms “a,” “an,” and “the,” include plural referents unless expressly and unequivocally limited to one referent. Thus, for example, reference to “a monomer” includes two or more monomers, and reference to “a PVTD” includes two or more PVTDs.

### Example 1

### Recombinant Expression and Purification of Fusion Proteins

This example demonstrates a preferred method for preparing recombinant collagen hybrid fusion proteins of this invention. Specifically it shows the use of Escherichia coli as host organism to express three fusion proteins identified herein as sequences RCH-1, RCH-2 and RCH-3 (Table W), each containing a segment of a human collagen THD sequence flanked by two or more PVTDs (FIG. 11).

### Fusion Protein Design

The RCH-1 fusion protein contains: a PfN capping domain with sequence PfN-28 (Table H), followed in frame by a PCoil domain with sequence PCoil-13 (Table I), followed in frame by a 111-amino acid sequence from the THD of human α1(II) collagen (residues 442-552 from sequence hCol-03, Table K), followed in frame by a PfC capping domain with sequence PfC-12 (Table J). An oligonucleotide sequence (i.d. RCHDNA-1, Table W) was designed, with a BamHI restriction site (GGATTC) at the 5′ end, followed in frame by a codon-optimised nucleotide sequence coding for the RCH-1 sequence, followed in frame by a double stop codon (TAATAA) and followed in frame by an EcoRI restriction site (GAATTC).

The RCH-2 fusion protein contains: a PfN capping domain with sequence PfN-80 (Table H), followed in frame by a PCoil domain with sequence PCoil-43 (Table I), followed in frame by a 360-amino acid modified sequence from the THD of human α1(II) collagen (residues 442-801 from sequence hCol-03, Table K, modified at positions 701-705 to the sequence ERGSP), followed in frame by a PfC capping domain with sequence PfC-04 (Table J). An oligonucleotide sequence (i.d. RCHDNA-2, Table W) was designed, with a BamHI restriction site (GGATTC) at the 5′ end, followed in frame by a codon-optimised nucleotide sequence coding for the RCH-2 sequence, followed in frame by a double stop codon (TAATAA) and followed in frame by an EcoRI restriction site (GAATTC).

The RCH-3 fusion protein contains: a PfN capping domain with sequence PfN-15 (Table H), followed in frame by a 252-amino acid sequence from the human α1(II) collagen THD (residues 400-651 from sequence hCol-03, Table K), followed in frame by a PfC capping domain with sequence PfC-61 (Table J). An oligonucleotide sequence (i.d. RCHDNA-3, Table W) was designed, with a BamHI restriction site (GGATTC) at the 5′ end, followed in frame by a codon-optimised nucleotide sequence coding for the RCH-3 sequence, followed in frame by a double stop codon (TAATAA) and followed in frame by an EcoRI restriction site (GAATTC).

### Expression and Purification

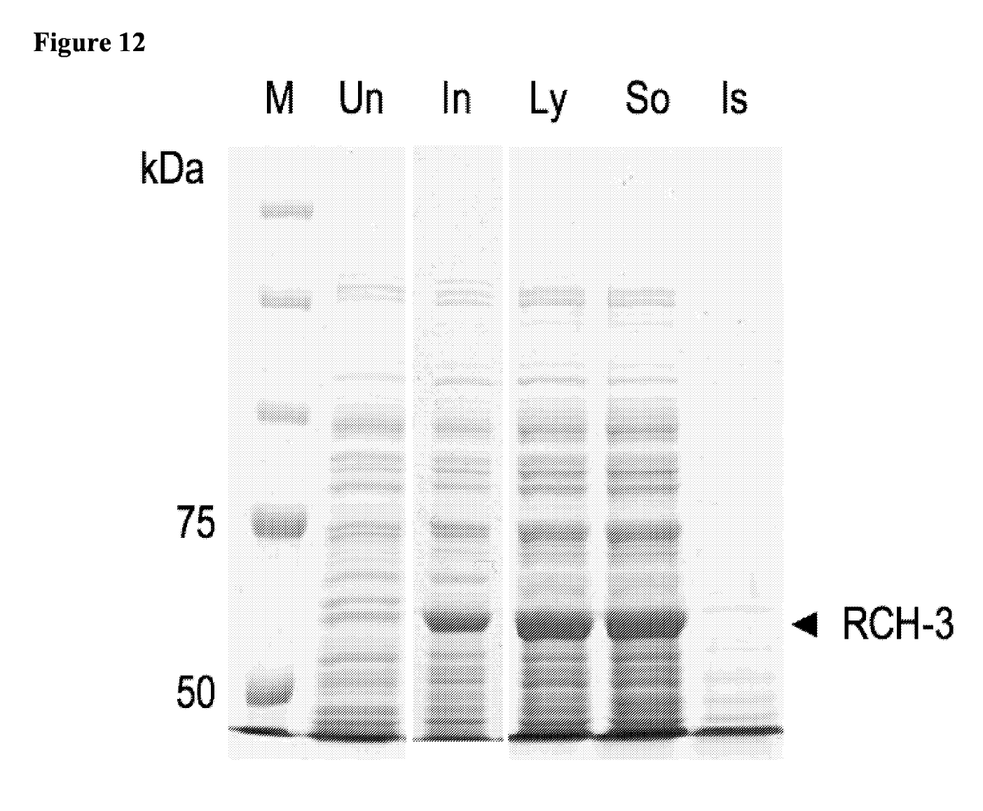

The designed DNA sequences RCHDNA-1, RCHDNA-2 and RCHDNA-3 (Table W), were synthesized commercially (GenScript Corporation, Piscataway, N.J., USA) and were cloned separately into a proprietary E. coli protein expression vector of the Protein Expression Facility of the Faculty of Life Sciences, University of Manchester. This vector (referred here as pHis) is a modification of the pET14b vector (originally developed by Novagen), incorporating codon-optimised sequences and an optimised multiple cloning site. All three sequences were cloned using the BamHI and EcoRI restriction sites. Each protein expression vector contained a start codon followed by a nucleotide sequence coding for an N-terminal His₆ tag, a thrombin cleavage site, and one of the fusion proteins (RCH-1, RCH-2 or RCH-3). All sequence elements in each vector were appropriately in frame. Competent E. coli cells were transformed with the different protein expression vectors and the respective proteins were expressed after induction with 0.5 mM isopropyl β-D-1-thiogalactopyranoside (IPTG) at 15° C. overnight (RCH-1), 0.1 mM IPTG at 12° C. for 68 hours (RCH-2), and 0.1 mM IPTG at 16° C. for 68 hours (RCH-3). Expression reached bulk yield values of 50-150 mg of recombinant protein per litre of culture, with longer induction times producing larger amounts of protein. The proteins were expressed predominantly in the soluble fraction (FIG. 12), and were purified by nickel-affinity chromatography on Ni-NTA agarose columns (QIAGEN, USA) followed by size-exclusion chromatography on a HiLoad 16/60 Superdex 200 preparative grade column (GE Healthcare, UK). Where required, samples were concentrated using Vivaspin 20 centrifugal concentrators (Sartorius Stedim Biotech, France). Sample purity was assessed by SDS-PAGE and the identities of the purified RCH-1, RCH-2 and RCH-3 proteins were confirmed by mass spectrometry: bands of interest were excised from the gel, digested with trypsin overnight at 37° C., and analysed by LC-MS/MS using a NanoAcquity LC system (Waters, Manchester, UK) coupled to a 4000 Q-TRAP spectrometer (Applied Biosystems, Framingham, Mass.).

### Example 2

### Quaternary Structure and Molecular Morphology of the Recombinant Proteins

Molecular weight determination by light scattering Proteins RCH-1, RCH-2 and RCH-3 were expressed and purified as described in example 1 and analyzed by size-exclusion chromatography followed by multiangle laser light scattering (MALLS) using a DAWN EOS instrument (Wyatt Technology, CA, USA). Light scattering allows measurement of the molecular weights of proteins in their native conformation. Both RCH-1 and RCH-2 were shown to be trimeric, consistently with the expected basic quaternary structure of collagens and collagen-like proteins. RCH-3 formed mainly large molecular-weight aggregates that could remain soluble at concentrations up to 0.5 mg/ml. Removal of these aggregates by size-exclusion chromatography made possible to isolate a low-molecular weight fraction that showed RCH-3 to be trimeric as well.

### Electron Microscopy

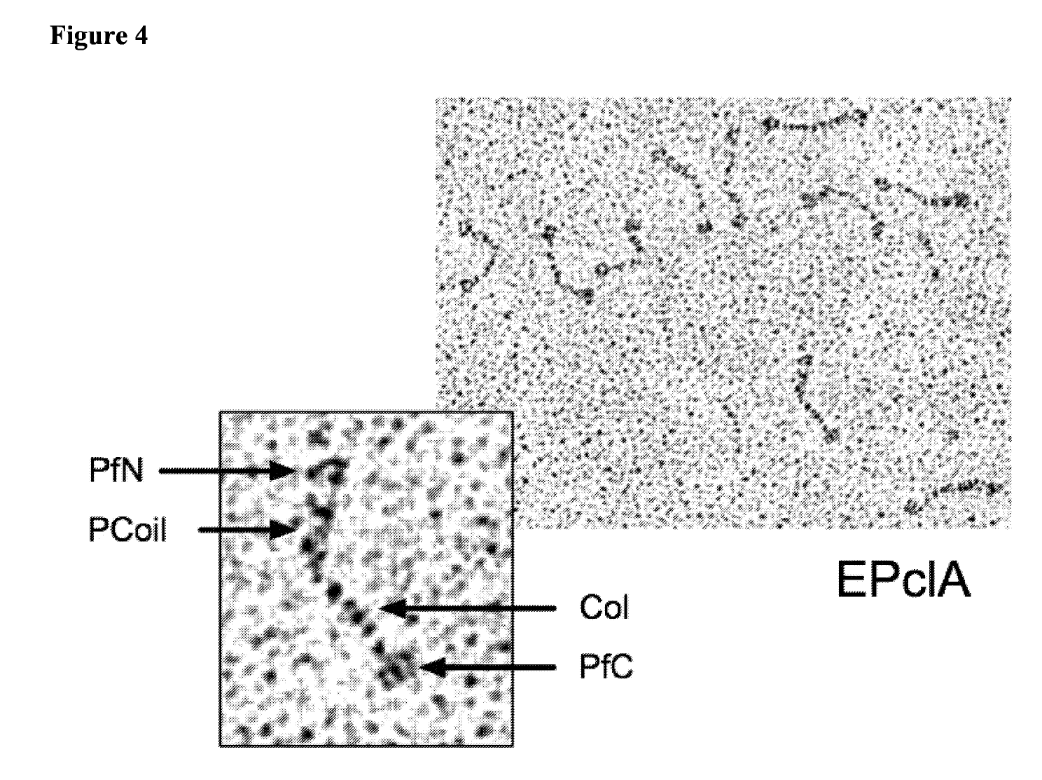

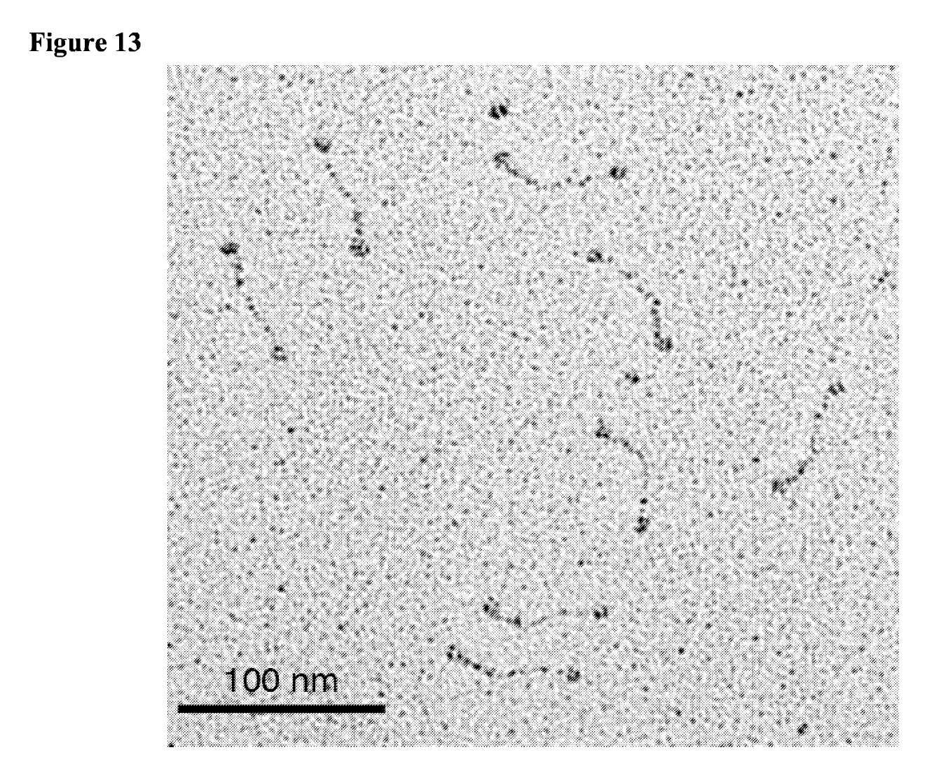

The molecular morphology of trimeric RCH-1, RCH-2 and RCH-3 was examined by rotary shadowing electron microscopy (EM). Samples were prepared following the mica sandwich technique (Mould et al., 1985: Mica sandwich technique for preparing macromolecules for rotary shadowing. J. Ultrastruct. Res., 91: 66-76) and examined in a FEI Tecnai Twin Transmission electron microscope operated at 1204 V. Images were recorded on a TVIPS F214 cooled CCD camera, and magnification was calibrated using a diffraction grating replica (Agar Scientific, Stansted, UK). The molecular morphology of RCH-1 (FIG. 13) is identical to that of the EPcIA protein (FIG. 4), with which it shares the same domain architecture. The RCH-1 protein has a dumbbell shape with two globular regions connected by a partially flexible stalk. The stalk contains the THD (fragment of human collagen) and a trimeric PCoil domain (a trimeric α-helical coiled coil). The two globular regions correspond to trimers of PfN and PfC domains, respectively.

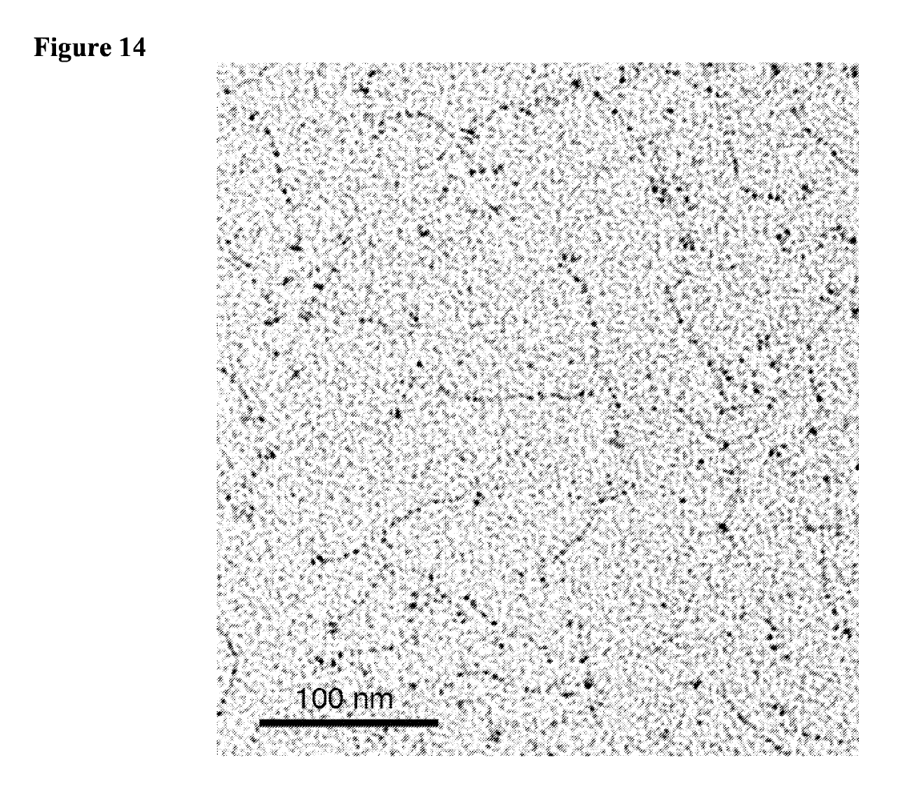

The molecular morphology of RCH-2 (FIG. 14) is also consistent with a longer collagen THD flanked by globular domains corresponding to PfN, PCoil, and PfC trimeric assemblies.

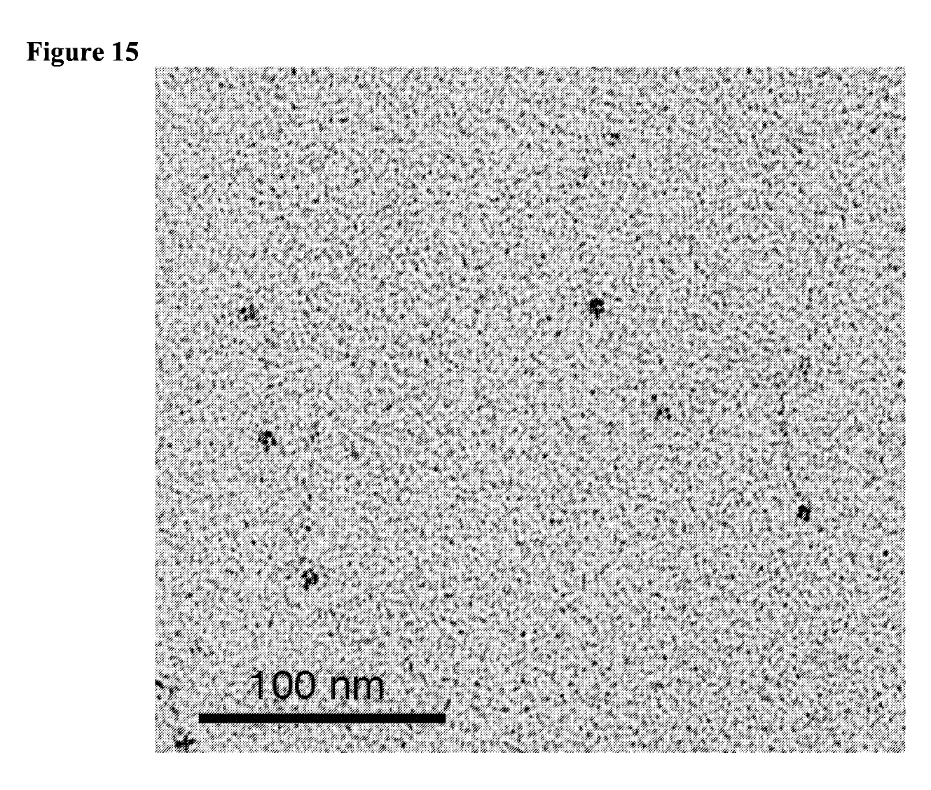

The molecular morphology of the low-molecular weight fraction of RCH-3 (FIG. 15) is consistent with a partially flexible collagen THD flanked by two globular regions, one being more prominent than the other in the electron microscopy images. The two globular regions correspond to trimers of PfN and PfC domains, respectively.

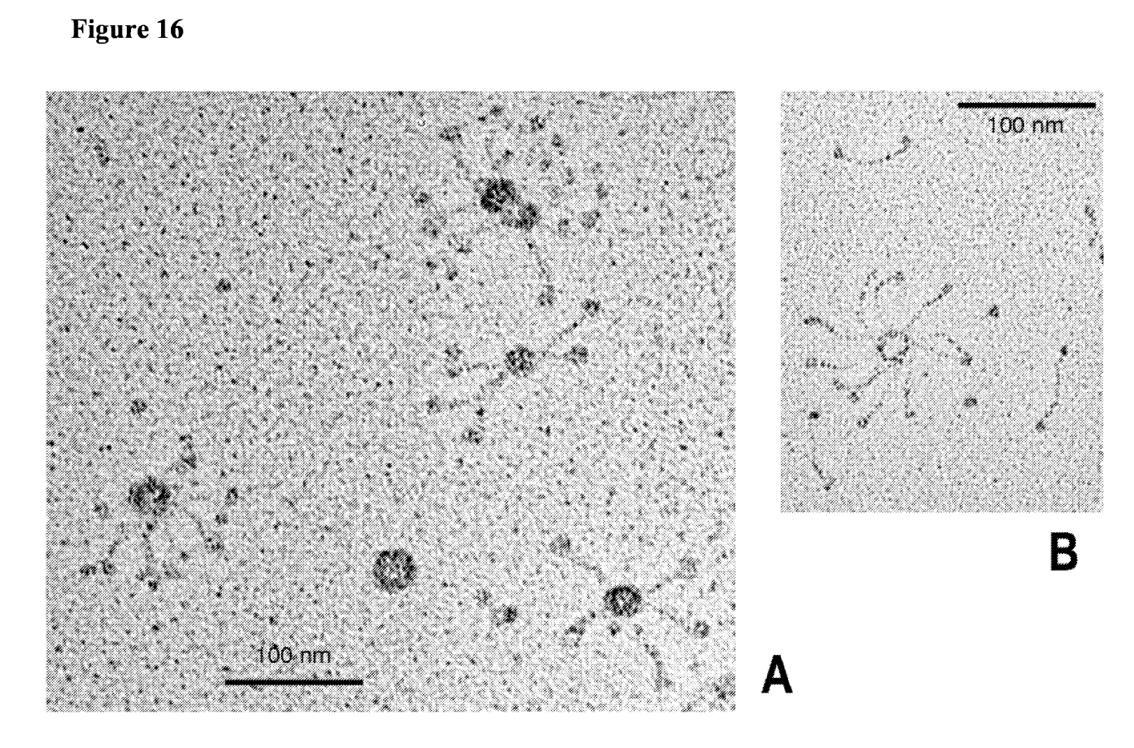

The molecular morphology of the high-molecular weight fraction of RCH-3 (FIG. 16A) reveals a dendrimer-like morphology for the high-molecular weight aggregates. These aggregates seem to occur through self-association of one of the globular regions, which would form the core of the dendrimer-like structures; from these central cores, the collagen THDs radiate and expose the globular regions on the other end at the periphery of the dendrimer-like structures. Exceptionally, similar structures have been observed in EM preparations of RCH-1 (FIG. 16B). The dendrimer-like structures from RCH-1 are consistent with oligomerization through the PfC globular regions and radial distribution of the THD, PCoil and PfN regions.

### Example 3

### Analysis of RCH-1 and RCH-2 by Circular Dichroism (CD)

### Conformational Analysis

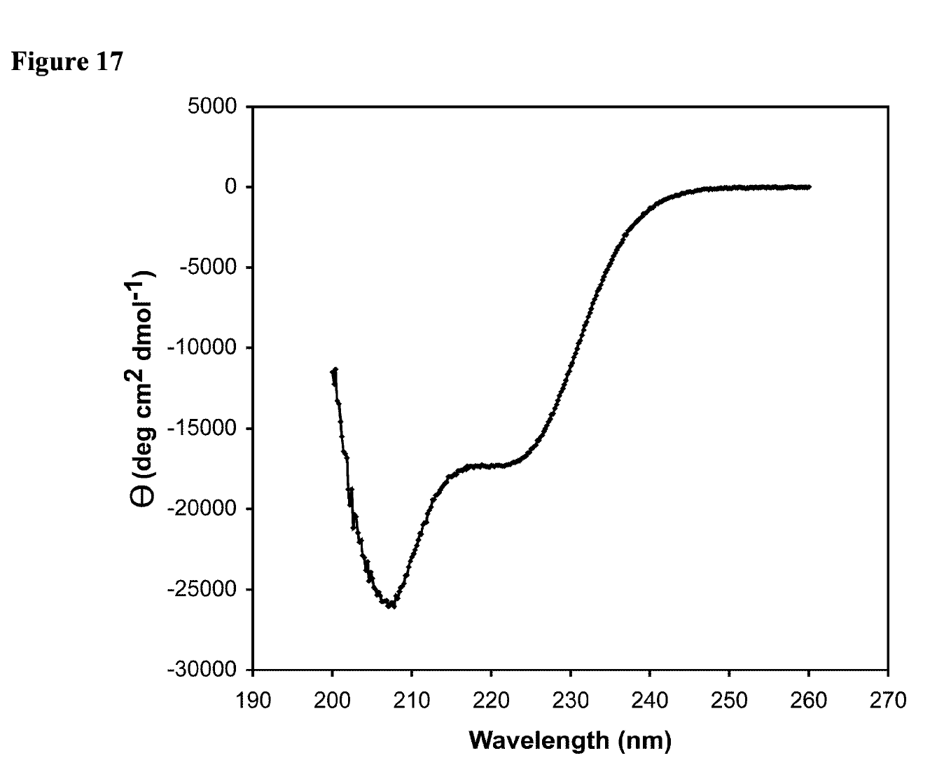

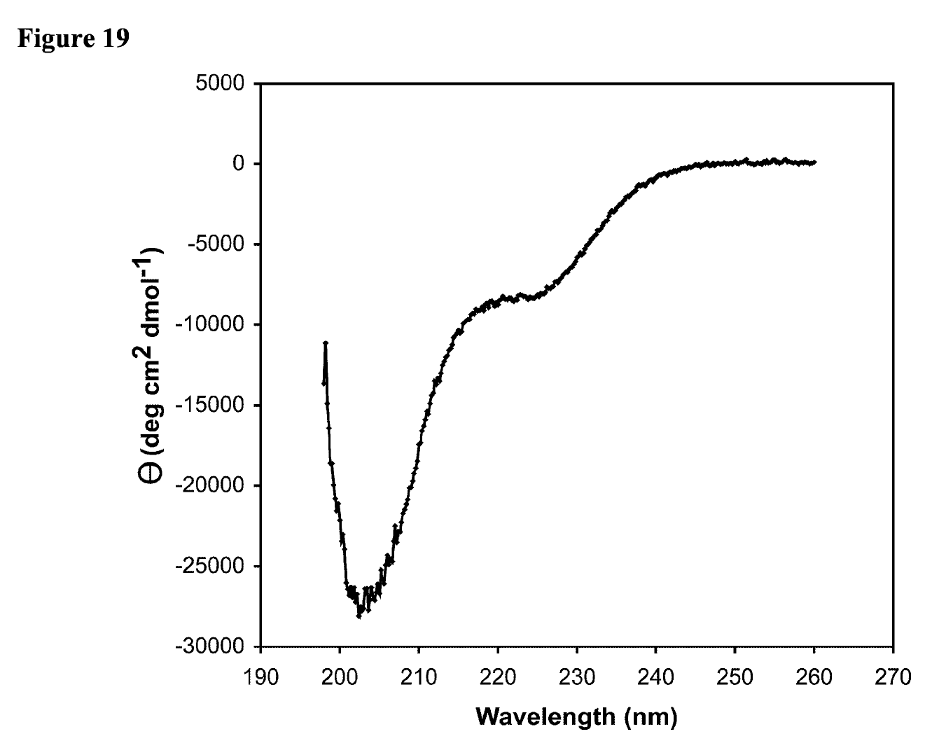

The secondary structure of the fusion proteins RCH-1 and RCH-2 was investigated by CD spectroscopy using a J-810 spectropolarimeter equipped with a Peltier temperature controller. Each protein sample was dissolved in 10 mM Tris-HCl pH 7.5, 150 mM NaCl, at concentrations of 0.5 mg/ml. Wavelength scans between 200 and 260 nm were performed for each protein at different temperatures, from 4° C. to 80° C., using a CD-matched quartz cuvette with a 0.5 mm path length. CD spectra at 4° C. for RCH-1 (FIG. 17) and RCH-2 (FIG. 19) are consistent with the combination of a collagen triple helix signal from the collagen THDs and an α-helical coiled-coil signal from the PCoil domains. The α-helical signal is much stronger in the RCH-1 spectrum (FIG. 17) than in the RCH-2 spectrum (FIG. 19).

The spectra of samples of RCH-1 heated above 45° C. did not show the characteristics of the collagen triple helical conformation and instead indicated an α-helical conformation. At that temperature the THD had unfolded while the α-helical structure of the PfN and PCoil domains remained largely intact. The same behaviour had been observed for the rEPcIA protein (FIG. 5A). Subsequent temperature increase above 65° C. eliminated the α-helical signal and the spectra indicated an unfolded structure.

The spectra of samples of RCH-2 heated above 35° C. did not show the characteristics of the collagen triple helical conformation and instead indicated an α-helical conformation, in a similar way to RCH-1 above. After increasing the temperature to 45° C. the α-helical signal disappeared completely and the spectra indicated an unfolded structure. Thus, the α-helical structure of the PfN and PCoil domains of RCH-2 is less stable than that of RCH-1 or rEPcIA.

### Thermal Transitions

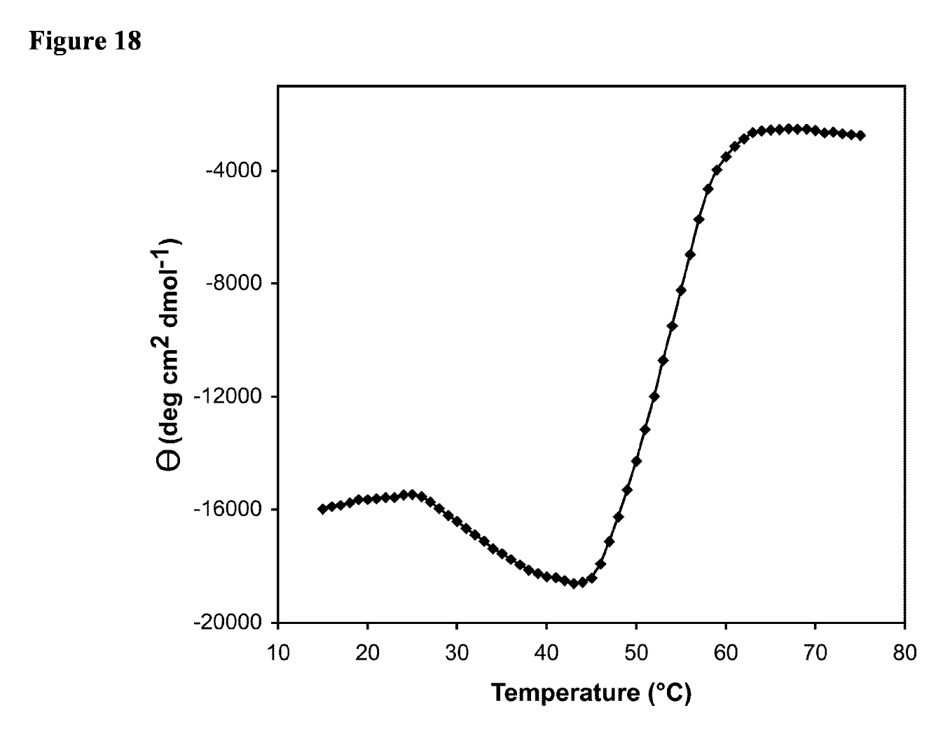

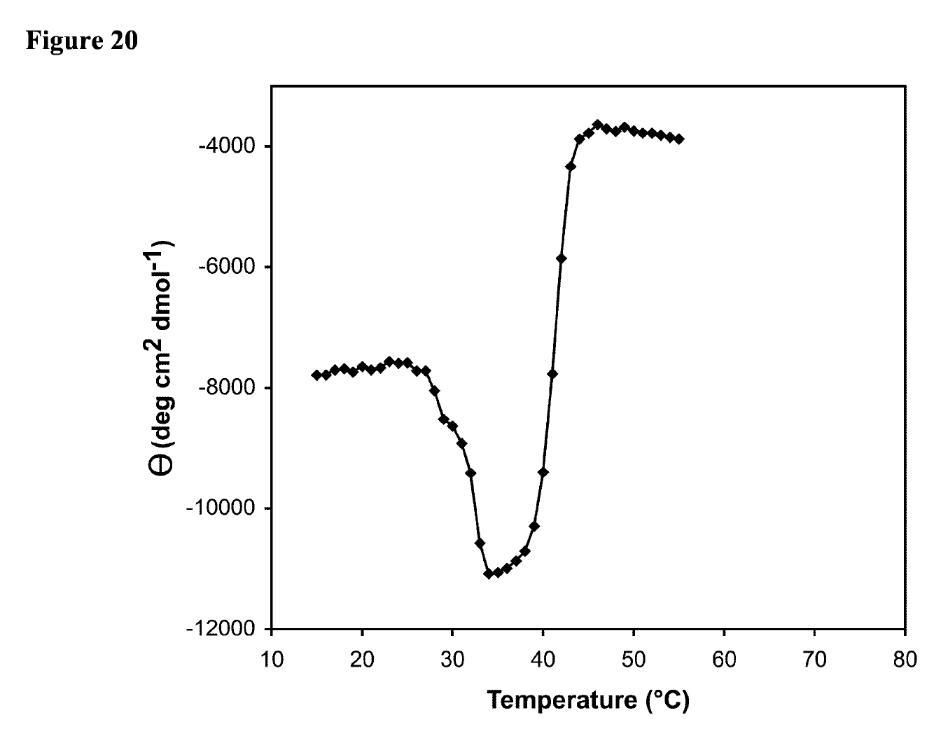

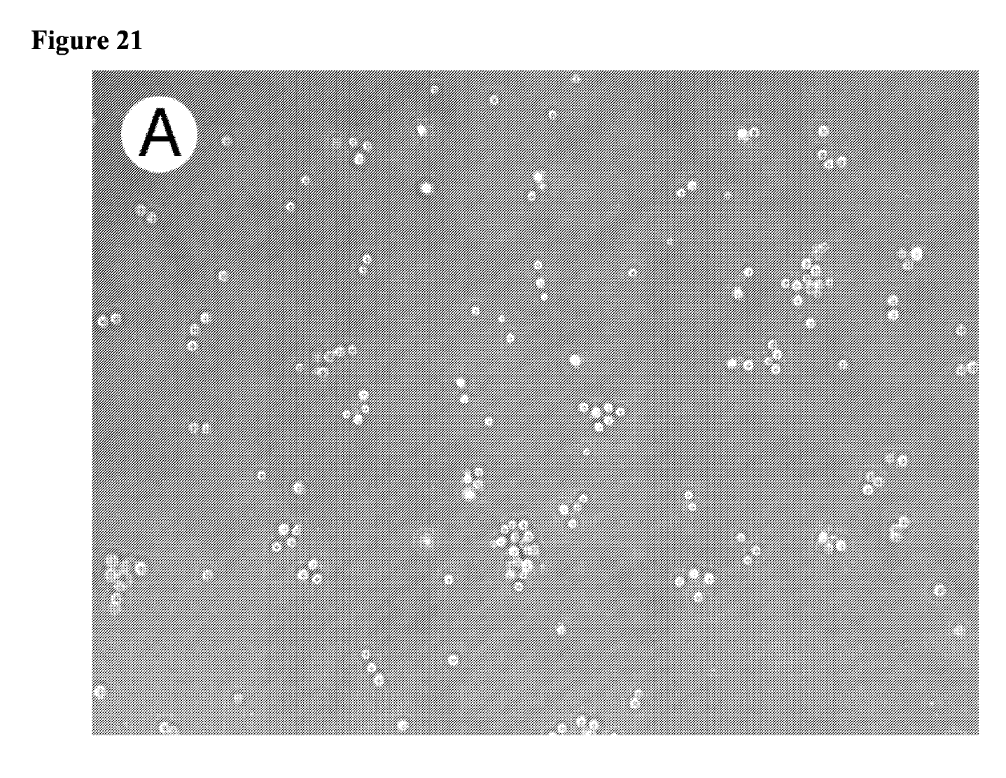

The thermal stability of RCH-1 and RCH-2 was investigated by monitoring the CD signal at 220 or 222 nm while varying the temperature (FIGS. 18 and 20). Samples (0.5 mg/ml in 10 mM Tris-HCl pH 7.5, 150 mM NaCl) were contained in a 0.5 mm quartz cuvette inside the J-810 spectropolarimeter and heated at a rate of 20° C./hour using the Peltier temperature controller; data were collected with 0.5 nm data pitch and 1 nm bandwidth. Both RCH-1 and RCH-2 show two transitions, the first one corresponding to the denaturation of the triple-helical structure of the collagen THDs and the second one corresponding to the denaturation of the α-helical coiled coil structure. Both collagen THDs denatured around the same temperature (32-33° C.), while the denaturation temperature of the α-helical coiled coil showed a significant difference between RCH-1 (53° C.) and RCH-2 (41° C.). The differences in thermal stability and in signal contribution to the overall CD spectrum (FIGS. 17 and 19) reflect unexpected conformational differences between the different PfN-PCoil domain combinations used in the RCH-1 and RCH-2 designs (FIG. 11).

The thermal unfolding of the collagen THDs of RCH-1 and RCH-2 above the first transition temperature was rapidly reversible: samples heated at 45° C. or 35° C. respectively and cooled down to 4° C. recovered CD spectra with the characteristic features of the collagen conformation. Samples heated above their second transition temperature did not recover rapidly their collagen conformation after cooling back to 4° C. Thus, the structural integrity of the capping domains, unaffected at the temperature of the first transition, appears critical for rapid reassembly of the collagen conformation of the RCHs. Nevertheless, samples heated above the second transition temperature did recover their collagen conformation, as shown by their CD spectra, after overnight incubation at 4° C.

### Example 4

### Cell Spreading Assays

### Fusion Protein Design

The three designed fusion proteins RCH-1, RCH-2 and RCH-3 contain natural or engineered integrin-binding sites (FIG. 11). The collagen sequence GFOGER (O: 4-hydroxyproline) is a high-affinity site for β1 integrins (Knight et al., 2000: The collagen-binding A-domains of integrins α1β1 and α2β1 recognize the same specific amino acid sequence, GFOGER, in native (triple-helical) collagens. J. Biol. Chem., 275: 35-40; Zhang et al., 2003: α11β1 integrin recognizes the GFOGER sequence in interstitial collagens. J. Biol. Chem., 278: 7270-7). Biomaterial formulations often use GFOGER peptides to promote cell adhesion (Reyes and Garcia, 2003: Engineering integrin-specific surfaces with a triple-helical collagen-mimetic peptide. J. Biomed. Mater. Res. A, 65: 511-23; Wojtowicz et al., 2010: Coating of biomaterial scaffolds with the collagen-mimetic peptide GFOGER for bone defect repair. Biomaterials 31: 2574-82). Hydroxylation is not critical, as the related GLPGER sequence mediates binding of prokaryotic collagen sequences to human integrin receptors (Caswell et al., 2008: Identification of the first prokaryotic collagen sequence motif that mediates binding to human collagen receptors, integrins α2β1 and α11β1. J. Biol. Chem., 283: 36168-75; Humtsoe et al., 2005: A streptococcal collagen-like protein interacts with the α2β1 integrin and induces intracellular signaling. J. Biol. Chem., 280: 13848-57).

### Cell Spreading Assays

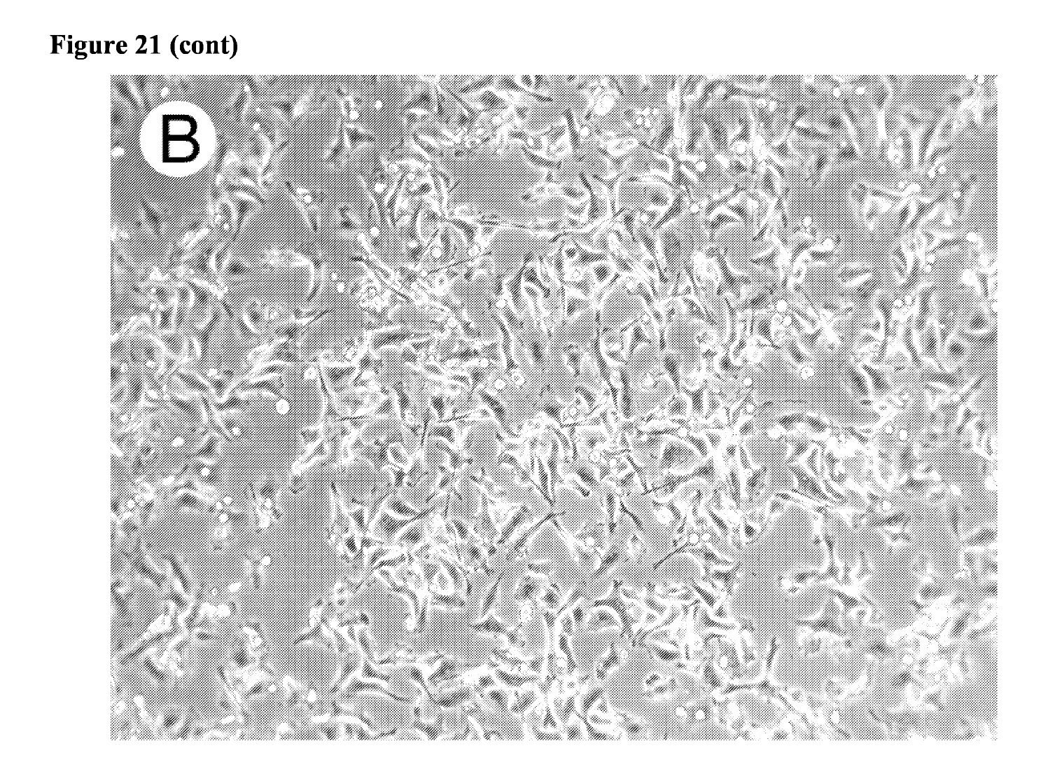

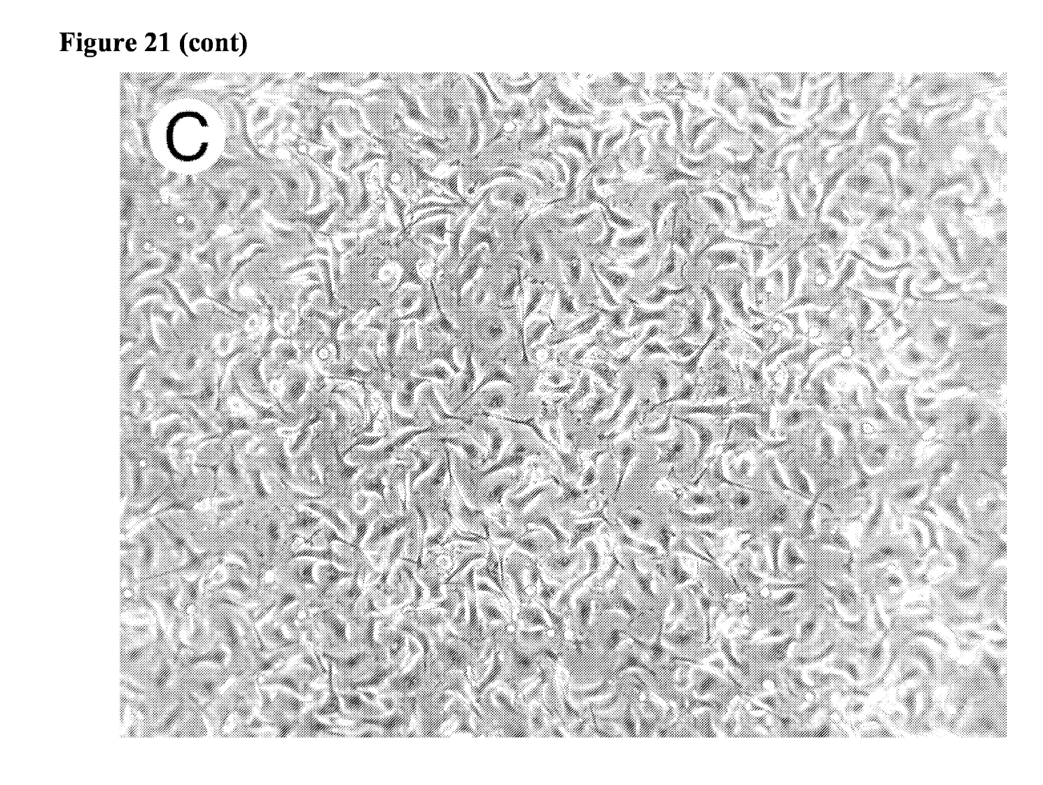

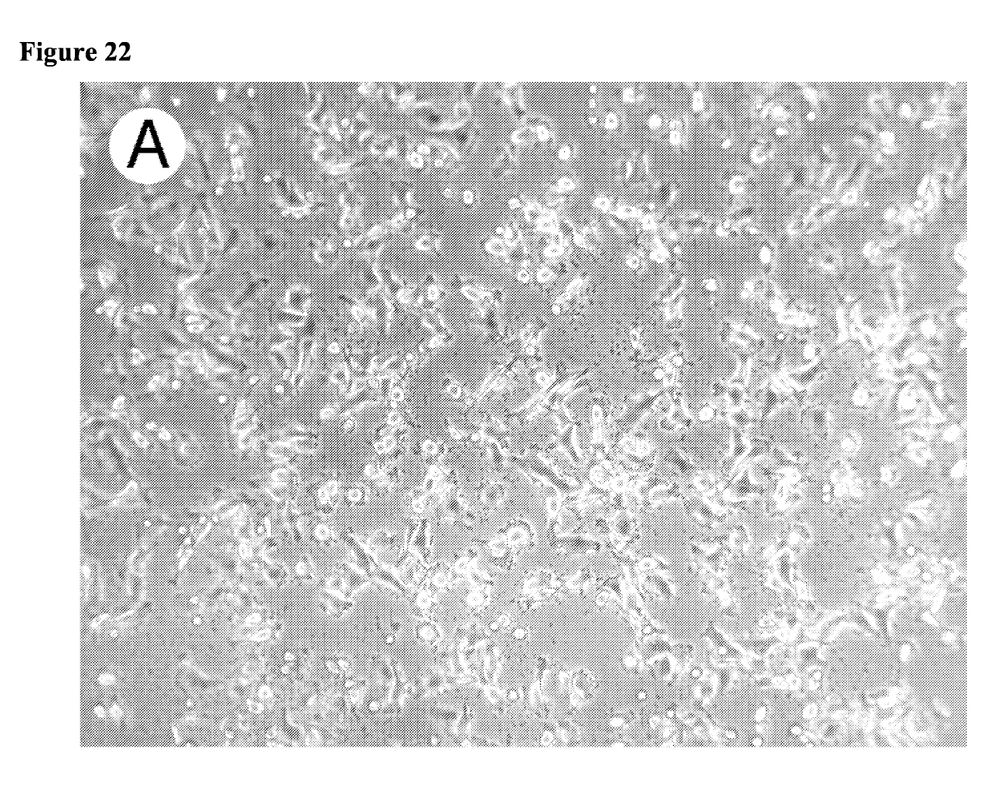

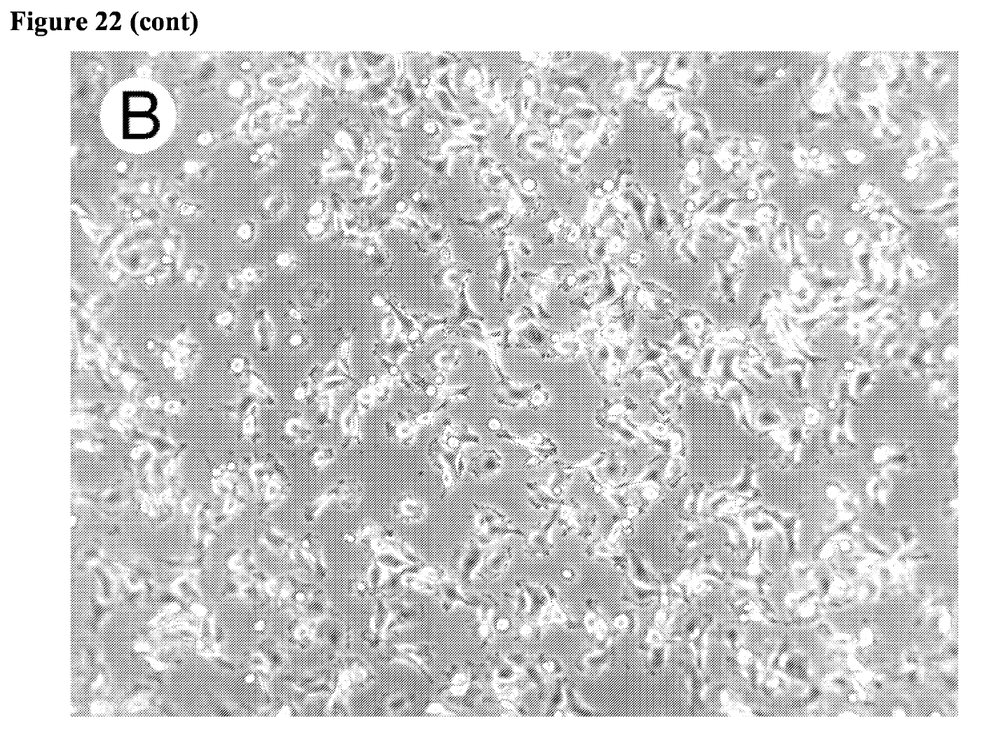

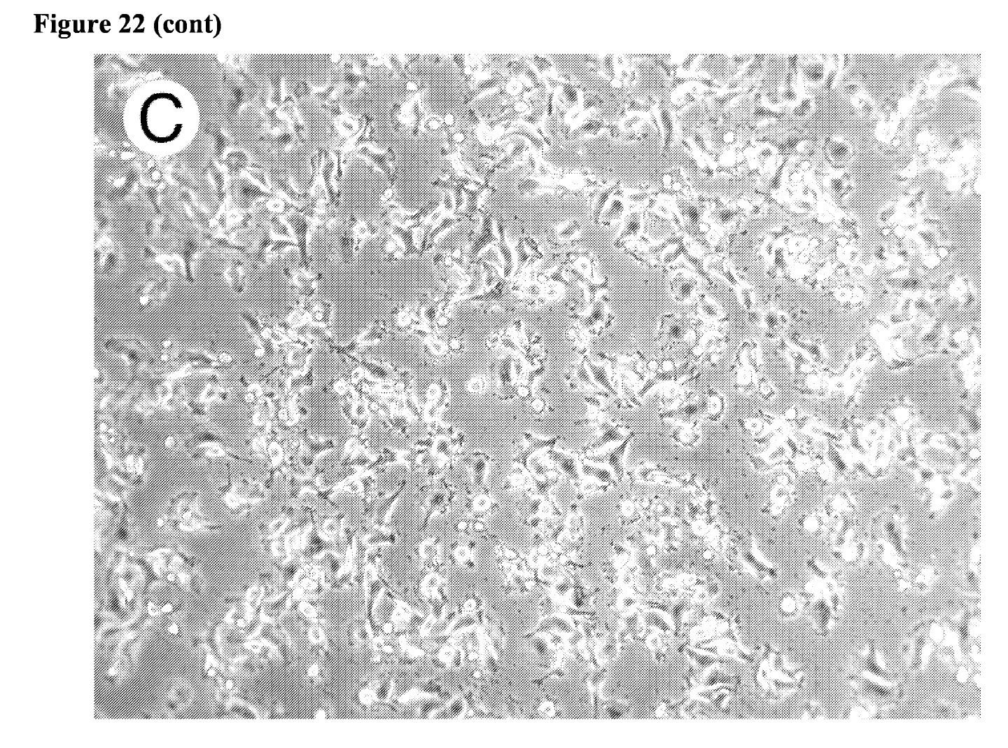

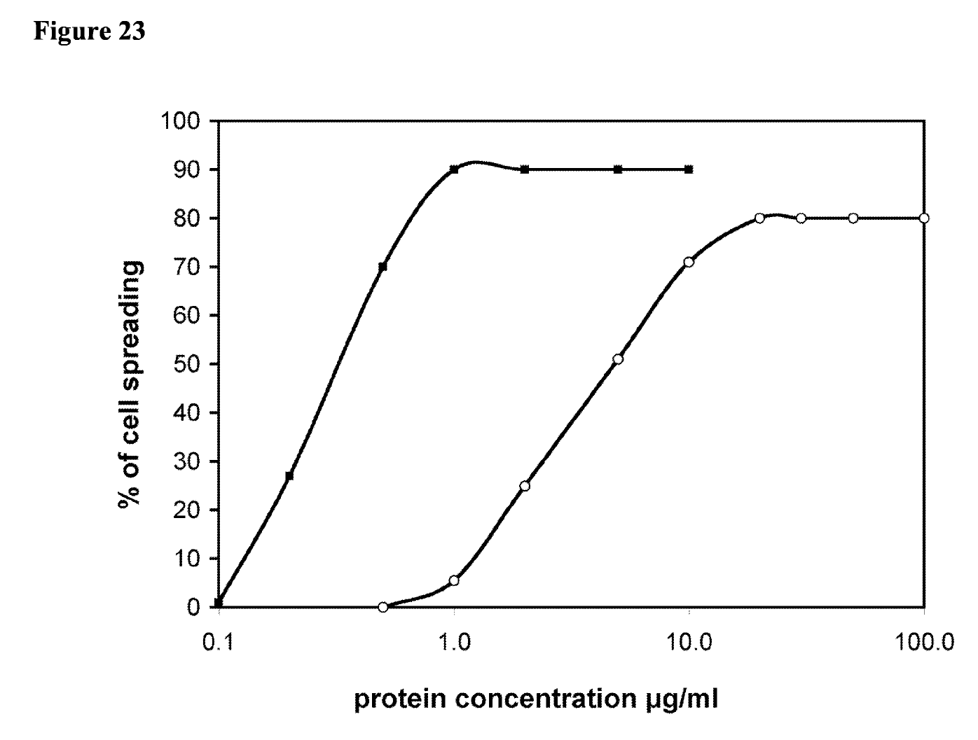

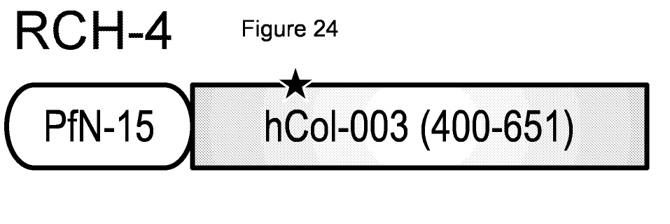

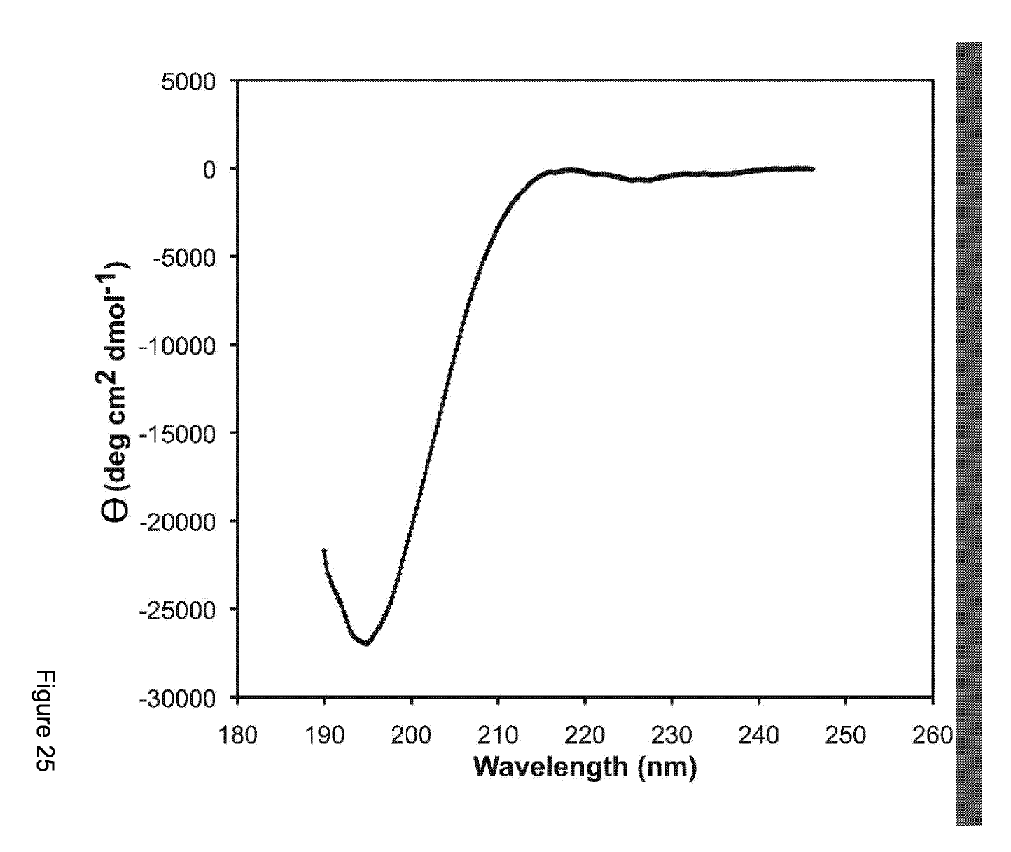

We have used the GFPGER sequence in the THDs of all three RCH fusion proteins to monitor their ability as substrates for cell adhesion. We used human fibrosarcoma HT1080 cells (human epithelial fibrosarcoma cell line), provided by Martin Humphries (University of Manchester, UK). Cells were cultured and maintained in DMEM supplemented with 10% fetal calf serum (Sigma), 2 mM L-Glutamine, and antibiotics (penicillin and streptomycin). Rat-tail collagen (Sigma) was used as positive control for cell spreading assays. Briefly, 96-well sterile tissue culture plates (Costar, Corning Inc, NY, USA) were coated for 1 hour at room temperature, or overnight at 4° C., with collagen or the RCH proteins at varying concentrations (1, 2, 5, 10, 20, 30, 50 and 100 μg/ml in phosphate buffered saline, PBS); rat-tail collagen at 10 μg/ml in PBS was used as positive control; plates treated with PBS (no protein present) or coated with the bacterial collagen protein EPcIA, were used as negative controls. After coating, plates were washed with PBS and blocked with 10 mg/ml heat-denatured (10 minutes at 85° C.) BSA, for 1 hour at room temperature. The excess of BSA was removed, plates washed with PBS, and 100 μl of HT1080 cell suspension (1×10⁵ cells/ml) were added and allowed to adhere for 90 minutes at 37° C. After this time, unattached cells were gently washed with PBS and attached cells were fixed with 100 μl of 5% glutaraldehyde (for 30 minutes at room temperature). Plates were then inspected with an inverted phase contrast microscope at 20×-100× magnifications. The percentage of spreading was measured by counting the proportion of spread cells. FIGS. 21, 22 and 23 show spreading of HT1080 cells on RCH-1 and RCH-3.

Prior to the experiments described in this example, we had already established that the bacterial protein EPcIA (FIG. 1) does not support cell adhesion of any of a variety of cell lines. EPcIA does not contain any GFPGER integrin binding site in its collagen domain. Thus, any adhesion properties of the RCH proteins are due to the integrin-binding sites in their sequences (our EPcIA data indicate that PfN, PCoil and PfC domains do not support adhesion). Interaction between GF/LP/OGER sequences and β1 integrins requires collagen to be in triple helical conformation; thus, positive cell adhesion also confirms the correct conformation of the collagen domains of our fusion proteins.

### Example 5

### Recombinant Fusion Protein with Only One Capping Domain

This example demonstrates that it is possible to prepare stable and soluble recombinant collagen hybrid fusion proteins of this invention where only one of the sides of the collagen sequence is flanked by a capping PVCTD.

### Fusion Protein Design

The RCH-4 fusion protein (FIG. 48) contains a PfN capping domain with sequence PfN-15 (Table H), followed in frame by a 252-amino acid sequence from the THD of human α1(II) collagen (residues 400-651 from sequence hCol-03, Table K). An oligonucleotide sequence was designed (i.d. RCHDNA-4, Table W) by PCR-amplification of the RCHDNA-3 sequence (Table W) truncated at the beginning of the PfC domain by using appropriate primers. The coding sequence terminates with a double stop codon after the human collagen sequence and therefore does not contain a C-terminal PVCTD. The oligonucleotide sequence RCHDNA-4 contains a 5′ BamHI restriction site (GGATTC) and a 3′ EcoRI restriction site (GAATTC).

### Expression and Purification

The designed DNA sequence RCHDNA-4 (Table W) was cloned into pHis, a proprietary E. coli protein expression vector of the Protein Expression Facility of the Faculty of Life Sciences, University of Manchester (see Example 1 for vector details). The RCHDNA-4 sequence was cloned using the BamHI and EcoRI restriction sites. The resulting protein expression vector contained a start codon followed by a nucleotide sequence coding for an N-terminal His₆ tag, a thrombin cleavage site, and the sequence coding for the fusion protein RCH-4. All sequence elements in the vector are appropriately in frame. Competent E. coli cells were transformed with the protein expression vector and the RCH-4 protein was expressed after induction with 0.1 mM isopropyl β-D-1-thiogalactopyranoside (IPTG) at 16° C. for 66 hours. Expression of RCH-4 protein reached bulk yield values of approximately 50 mg of recombinant protein per litre of culture, similar to those of other RCHs (see Example 1). The protein was detected mainly (>90%) in the soluble fraction. RCH-4 was purified by nickel-affinity chromatography on Ni-NTA agarose columns (QIAGEN, USA) followed by size-exclusion chromatography on a HiLoad 16/60 Superdex 200 preparative grade column (GE Healthcare, UK). Sample purity was assessed by SDS-PAGE and the identity of the RCH-4 protein was confirmed by mass spectrometry. When needed, purified RCH-4 protein was concentrated using Vivaspin 20 centrifugal concentrators (Sartorius Stedim Biotech, France).

### Molecular Weight Determination by Light Scattering

Purified RCH-4 was analyzed by size-exclusion chromatography (SEC) followed by multiangle laser light scattering (MALLS) using a DAWN EOS instrument (Wyatt Technology, CA, USA). The MALLS analysis showed RCH-4 to be trimeric, and not to form the large molecular-weight aggregates that were predominant in RCH-3. Thus, the aggregation of RCH-3 into dendrimer-like macro-structures was induced by the presence of its 94-amino acid C-terminal PVCTD (sequence PfC-61, Table J).

### Conformational Analysis of RCH-4

The secondary structure of the fusion protein RCH-4 was investigated by CD spectroscopy using a J-810 spectropolarimeter equipped with a Peltier temperature controller. The RCH-4 protein was dissolved in 5 mM Tris-HCl pH 7.5, 150 mM NaCl, at a concentration of 0.13 mg/ml. A wavelength scan was performed between 190 and 250 nm at different temperatures, using a CD-matched quartz cuvette with a 1 mm path length. The CD spectra at 4° C. for RCH-4 (Table B) is consistent with a collagen triple helix signal from the collagen THD, with a small maximum at 218 nm and a deep minimum at 195 nm. The spectra of a RCH-4 sample heated above 45° C. did not show the characteristics of the collagen triple helical conformation.

### Thermal Transitions

The thermal stability of RCH-4 was investigated by monitoring the CD signal at 220 nm while varying the temperature. The sample (1.3 mg/ml in 10 mM Tris-HCl pH 7.5, 150 mM NaCl) was contained in a 1 mm quartz cuvette inside the J-810 spectropolarimeter and heated at a rate of 20° C./hour using the Peltier temperature controller; data were collected with 0.5 nm data pitch and 1 nm bandwidth. RCH-4 shows a transition at 22° C. corresponding to the denaturation of the triple helical structure of the collagen THD.

### Example 6

### Liophylization and Re-Solubilization of RCH-1

This example demonstrates the suitability of our RCHs for usual preparation protocols used for commercially available collagen proteins, where the collagens are lyophylized at the source for storage and commercial delivery and are then re-solubilised by the end user in appropriate buffers, prior to their use in diverse applications.

Purified samples of RCH-1 in 20 mM Tris-HCl pH 7.9, 150 mM NaCl, 1 mM EDTA buffer were transferred into MW CO 12-14,000 dialysis tubing (Medicell International Ltd.) and sealed at both ends for dialysis overnight on a Rodwell Monostir (200/250V) against MilliQ H₂O. Dialysed samples were analysed by SDS-PAGE to confirm the presence of the intact RCH-1 protein. The secondary structure of RCH-1 in water was also confirmed by CD spectroscopy.

Samples of RCH-1 dialysed into water were freeze-dried using a Heto Lyolab3000 lyophillizer. Freeze-dried samples were suitable for storage at −20° C. (short-term) or −80° C. (long-term). To test the limits of solubility in water, a sample of freeze-dried RCH-1 was weighted in a TR-scale (Denver Instrument Company) and then re-solubilized in the smallest possible volume of MilliQ H₂O to obtain a highly concentrated sample of RCH-1. MilliQ H₂O was added in 2 μl droplets until complete dissolution was observed. A concentration of approximately 40 mg/ml was achieved after adding 85 μl of H₂O to a 3.4 mg sample of lyophilised RCH-1.

### Example 7

### Large-Scale Production of RCH-1 Using a Pilot Fermentation Run

This example demonstrates the suitability of our RCHs for large-scale production using 20-litre fermentation equipment (Applikon Biotechnology).

A 5 ml sample of LB medium with ampicillin was inoculated with a single colony of E. coli cells expressing the RCH-1, and then incubated at 37° C. for 7 hours. Two 400 ml flasks of LB medium with ampicillin were then inoculated with 0.4 ml (0.1%) of the 7-hour culture and incubated overnight at 37° C. Medium for the 20-litre fermentation was prepared in as follows: Trypton (200 g), Yeast extract (200 g) and NaCl (200 g) were dissolved in water up to a final volume of 20 litres. Ampicillin was added to a final concentration of 50 μg/ml and the pH was adjusted to 7.0. The 20-litre LB medium was inoculated with 400 ml (2%) of the overnight culture (OD₆₀₀=0.059) and incubated at 37° C. for 1 h 50 min to a OD₆₀₀=0.611. The culture was then cooled to 25° C. for 10 minutes, and 20 ml of 100 mM IPTG were added to the fermentor (final concentration of IPTG was 0.5 mM). The culture was maintained at 16° C. and pH 7.0 for 18 hours after induction.

Cells were collected by centrifugation using a JLA-8100 rotor at 4° C., at 5000 rpm for 15 minutes in 6 1-litre bottles. Cells were then washed 6 times with 45 ml of 10 mM Tris-HCl pH 7.5, 150 mM NaCl. Subsequently the cells were weighted (80 g) and stored at −80° C. for later use.

To estimate the level of RCH-1 production a 1 g pellet of cells was allowed to thaw on ice for about 15 minutes before adding 10 ml of lysis buffer and one tablet of EDTA-free protease inhibitor cocktail (Complete Mini). The cells were then gently resuspended and sonicated on ice using a Sonopuls with a T13 probe (Bandelin) until viscosity was visibly reduced. The lysate was then centrifuged at 4° C. for 15 minutes at 17,000 RPM using an Avanti J-E centrifuge with a JA-17 Rotor (Beckman Coulter). Total and soluble protein content were analysed by SDS-PAGE, which showed that over-expressed RCHs was largely collected in the soluble fraction. From the amount of protein recovered by a small-scale nickel-affinity purification it was possible to estimate the bulk production of RCH-1 in the 20-litre pilot fermentation as approximately 0.8-1 mg/ml, which doubles the best yield obtained in 1-litre flask culture (0.3-0.5 mg/ml).

During our investigation on these collagen-like proteins it was discovered that the triple-helical domain of the bacteriophage collagen-like protein EPcIA has a very high melting temperature, 42° C. (FIGS. 3 and 5), much higher that what could have been expected from its relatively short sequence (111 amino acids) and the lack of prolyl hydroxylation or glycosylation. It was also discovered that the triple helical collagen domain recovered its native conformation very quickly after thermal denaturation. Recombinant expression of the EPcIA protein in E. coli demonstrated that this protein is highly soluble and does not accumulate in insoluble inclusion bodies. These three properties would make EPcIA itself an interesting molecule for further development into biomaterial applications. However, it was hypothesized that the molecular architecture of EPcIA could be exploited for the design of new proteins containing human collagen sequences that could be expressed successfully in E. coli with high yields, good solubility, and improved thermal stability.

Some of the non-collagenous capping domains present in EPcIA (PfN, PfC, PCoil, FIG. 1) were contributing to maintain these prokaryotic collagen proteins in soluble form, were contributing to the increase in the thermal stability of the collagen triple helical domain, and were facilitating the refolding of the collagen triple helical domains after thermal denaturation. The data indicates that the PfC, PfN and PCoil regions are trimerization domains that play equivalent roles to the N- and C-terminal propeptides in fibrillar collagens. They would act as registration peptides, maintaining these collagen-like proteins in soluble form and contributing to the thermal stability of the collagen regions.

### SUMMARY

Herein, the inventors designed a novel approach where the PfC, PfN and PCoil domains from bacteriophage collagen-like proteins could be used as capping domains for the expression of human or mammalian triple-helical collagen sequences in E. coli. In recombinant protein designs, these domains are fused in frame with heterologous collagen sequences of human origin, to assist them in their proper folding, solubility, and thermal stability. The phage capping domains would help in maintaining solubility and would compensate in part for the lack of prolyl hydroxylation, providing enough stabilization to overcome complete proteolytic degradation during protein expression. Due to its unique structure, triple helical collagen is highly resistant to proteolysis; however, monomer chains are largely unfolded and therefore susceptible to degradation in prokaryotes (that do not have the endoplasmic reticulum into which secrete the newly synthesized polypeptide chains). Successful expression of soluble human or mammalian collagen sequences in E. coli is therefore dependent on how quickly the recombinant protein can adopt the triple helical form before the individual chains are degraded by proteolysis. The capping domains of phage collagen-like proteins seem to be exceptionally effective in that task.

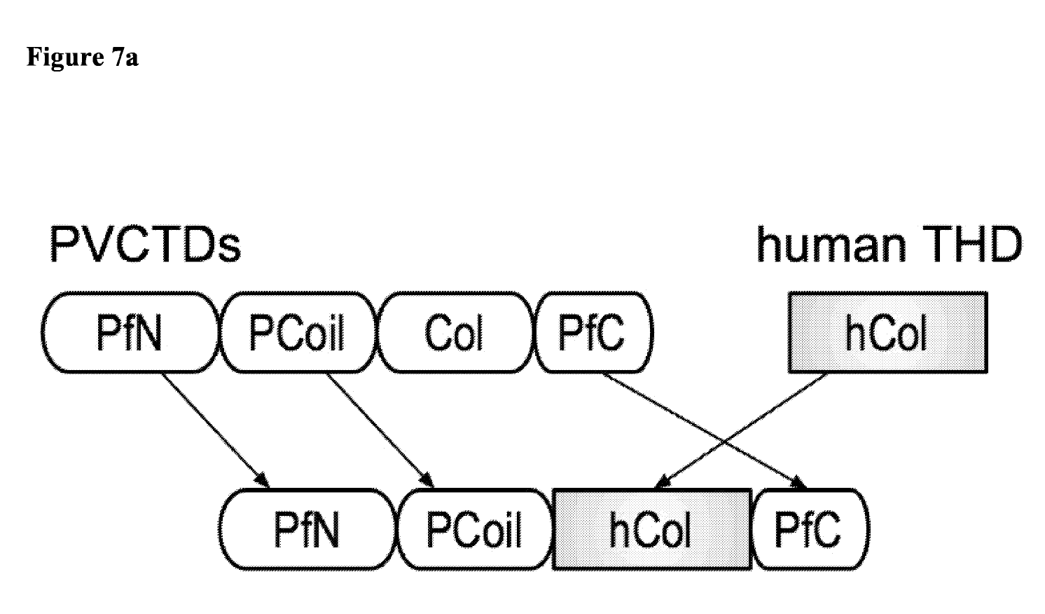

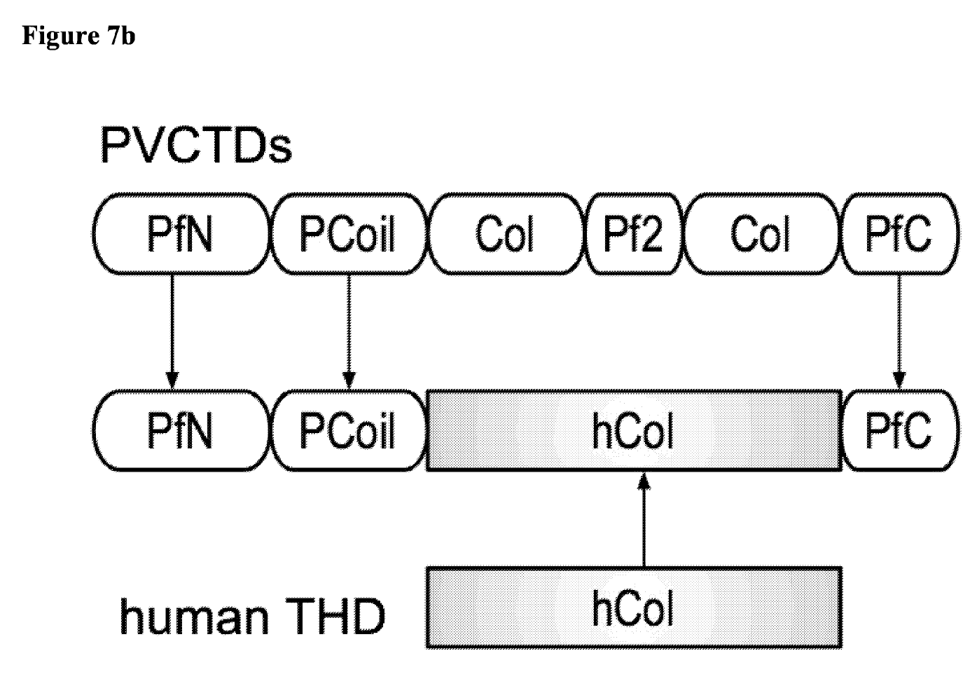

To test the hypothesis we generated several recombinant human collagens (rhCs) where the collagen-like sequence of a bacterial or phage collagen-like protein was exchanged with a sequence from a human collagen (FIG. 7; FIG. 11). Successful expression of these rhCs in E. coli was achieved entirely expressed as soluble proteins, with no evidence of inclusion body formation (FIG. 12). Solubility in water of purified rhCs at least up to 40 mg/ml was shown. Their molecular morphology was consistent with a folded collagen conformation (FIGS. 13-20) that contained correctly folded cell-binding sites that supported cell-adhesion via eukaryotic receptor recognition (FIGS. 21-23). The RHCs containing both N-terminal and C-terminal capping domains showed melting temperatures of 32-33° C. for the triple helical human collagen domains. Their thermal stability is higher than that of much longer, non-hydroxylated type I collagen sequences produced (in much smaller amounts) in transgenic plants. Thus, the phage capping domains significantly stabilize the triple helical domains of in-frame human collagen sequences.

Therefore domains from bacteriophage collagen-like proteins can contribute to the solubility and stability of collagen triple helical domains, including those with human sequences.

Lengthy table referenced here
US20130237486A1-20130912-T00001
Please refer to the end of the specification for access instructions.

Lengthy table referenced here
US20130237486A1-20130912-T00002
Please refer to the end of the specification for access instructions.

Lengthy table referenced here
US20130237486A1-20130912-T00003
Please refer to the end of the specification for access instructions.

Lengthy table referenced here
US20130237486A1-20130912-T00004
Please refer to the end of the specification for access instructions.

Lengthy table referenced here
US20130237486A1-20130912-T00005
Please refer to the end of the specification for access instructions.

Lengthy table referenced here
US20130237486A1-20130912-T00006
Please refer to the end of the specification for access instructions.

Lengthy table referenced here
US20130237486A1-20130912-T00007
Please refer to the end of the specification for access instructions.

Lengthy table referenced here
US20130237486A1-20130912-T00008
Please refer to the end of the specification for access instructions.

Lengthy table referenced here
US20130237486A1-20130912-T00009
Please refer to the end of the specification for access instructions.

Lengthy table referenced here
US20130237486A1-20130912-T00010
Please refer to the end of the specification for access instructions.

Lengthy table referenced here
US20130237486A1-20130912-T00011
Please refer to the end of the specification for access instructions.

Lengthy table referenced here
US20130237486A1-20130912-T00012
Please refer to the end of the specification for access instructions.

Lengthy table referenced here
US20130237486A1-20130912-T00013
Please refer to the end of the specification for access instructions.

Lengthy table referenced here
US20130237486A1-20130912-T00014
Please refer to the end of the specification for access instructions.

Lengthy table referenced here
US20130237486A1-20130912-T00015
Please refer to the end of the specification for access instructions.

Lengthy table referenced here
US20130237486A1-20130912-T00016
Please refer to the end of the specification for access instructions.

Lengthy table referenced here
US20130237486A1-20130912-T00017
Please refer to the end of the specification for access instructions.

Lengthy table referenced here
US20130237486A1-20130912-T00018
Please refer to the end of the specification for access instructions.

Lengthy table referenced here
US20130237486A1-20130912-T00019
Please refer to the end of the specification for access instructions.

Lengthy table referenced here
US20130237486A1-20130912-T00020
Please refer to the end of the specification for access instructions.

Lengthy table referenced here
US20130237486A1-20130912-T00021
Please refer to the end of the specification for access instructions.

Lengthy table referenced here
US20130237486A1-20130912-T00022
Please refer to the end of the specification for access instructions.

Lengthy table referenced here
US20130237486A1-20130912-T00023
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20130237486A1). An electronic copy of the table will also be available from the USPTO upon request and payment of the fee set forth in 37 CFR 1.19(b)(3).

1. A trimeric fusion protein comprising three polypeptide chains, wherein each polypeptide chain comprises a eukaryotic collagen or collagen-like domain and a prokaryotic or viral trimerisation domain (PVTD).

2. A fusion protein according to claim 1 having one or more of the following, independently selected, properties:
a) a melting temperature of between 34° C. and 60° C., preferably between 34° C. and 59° C., more preferably between 34° C. and 58° C., 57° C., 56° C., 55° C., 54° C., 53° C., 52° C., 51° C., 50° C., 49° C., 48° C., 47° C., 46° C., or 45° C., more preferably between 38° C. and 44° C., more preferably between 39° C. and 43° C., more preferably at least 40° C., 41° C. or 42° C.;
b) solubility of at least 25, at least 30, at least 31, at least 32, at least 33, at least 34, at least 35, at least 36, at least 37, at least 38, at least 39, or at least 40 mg/ml;
c) is comprised of one or more fusion polypeptides which are substantially resistant to proteolytic degradation by host enzymes when expressed in prokaryotic cells; and
d) exhibit improved ability to refold after denaturation into a collagen or collagen-like structure.

3. A trimeric protein according to claim 1, wherein the fusion protein forms trimers by association of the three polypeptide chains, and preferably forms a triple-helical structure.

4. A fusion protein according to claim 1 wherein two or more of the three polypeptide chains are the same as each other or different.

5. A fusion polypeptide comprising a eukaryotic collagen or collagen-like domain and a PVTD.

6. A fusion protein according to claim 1, wherein the PVTD is derived from a collagen or collagen-like protein.

7. A fusion protein according to claim 1, wherein the PVTD may be provided:
i) within a eukaryotic collagen or collagen-like domain; and/or
ii) flanking one or both ends of a eukaryotic collagen or collagen-like domain; and/or
iii) within non-eukaryotic collagen or collagen-like domain of the fusion polypeptide and/or flanking one or both ends thereof.

8. A fusion protein according to claim 1, wherein the PVTD comprises one or more functional sequences independently selected from the group consisting of stabilization sequences, binding sites, cleavage sites, and linkage sites.

9. A fusion protein according to claim 1, wherein the eukaryotic collagen or collagen-like domain is derived from vertebrate collagen or collagen-like proteins, preferably mammalian, ruminate, fish, or preferably human.

10. A fusion protein according to claim 1 wherein the eukaryotic collagen or collagen-like domain of the fusion protein or polypeptide is composed of two or more heterologous collagen or collagen-like domains operably linked to form a single collagen or collagen-like domain.

11. A fusion protein according to claim 10, wherein more than one eukaryotic collagen or collagen-like domains is present, and wherein one or more or all may be chimeric.

12. A fusion protein according to claim 1, wherein the eukaryotic collagen or collagen-like domain comprises:
i) a human fibrillar collagen chain selected from a1(I), 2(I), a1(II) and a1(III);
ii) a eukaryotic collagen or collagen-like domain comprising a sequence selected from the group consisting of sequences hCol-01 to hCol-89 of Tables K and L;
iii) a sequence consisting of a sequence selected from the groups consisting of the human collagen sequences any of hCol-01 to hCol-49 of Table K and the collagen-like domains of any of hCol-50 to hCol-89 of Table L;
iv) a domain or sequence having at least 50%, 60%, 70%, 80%, 90%, 91%, 92%, 93%, 94%, 95%, 96%, 97%, 98% or 99% sequence identity with a sequence of i), ii) or iii); or
v) fragments, variants or derivatives of a sequence of any of i) to iv).

13. A fusion protein according to claim 1, comprising one or more THDs (triple helical domains), either in tandem or separated by one or more PVTDs or other sequences.

14. A fusion protein according to claim 1, further comprising one or more functional domains, selected from the group consisting of binding sites, cleavage sites, linkage sites, and trimerisation sites.

15. A fusion protein according to claim 1 wherein a eukaryotic collagen or collagen-like domain may be independently selected from the group consisting of vertebrate, mammalian, ruminate, fish, or human collagen or collagen-like proteins.

16. A fusion protein according to claim 1, wherein the PVTD is derived from a bacterial source, preferably gram negative bacteria, preferably pathogenic E. coli, preferably E. coli strain O157:H7.

17. A fusion protein according to claim 1, wherein the PVTD may be:
i) a PVTD of any of EPcIA-001 to EPcIA-142 of Table A, any of EPcIB-001 to EPcIB-021 of Table B, any of EPcIC-001 to EPcIC-005 of Table C, or EPcID-001 of Table D, any of PfN-01 to PfN-86 of Table H, any of PCoil-01 to PCoil-46 of Table I, any of PfC-01 to PfC-61 of Table J, and a Pf2 sequence, preferably one of the Pf2 domains in sequences any of EPcIB-001 to EPcIB-021 of Table B;
ii) having an amino acid sequence having at least 50%, 60%, 70%, 80%, 90%, 91%, 92%, 93%, 94%, 95%, 96%, 97%, 98% or 99% sequence identity with a PVTD of i);
iii) encoded by a nucleic acid selected from the group consisting of sequences of Table E to G and M to R or a nucleic acid sequence having at least 50%, 60%, 70%, 80%, 90%, 91%, 92%, 93%, 94%, 95%, 96%, 97%, 98% or 99% sequence thereto; or
iv) a fragment or derivative of an afore-mentioned sequence which functions as a PVTD

18. A fusion protein according to claim 1, wherein the fusion protein comprises two or more PVTDs, the combination of PVTD's being selected from:
i) one or more sequences independently selected from the group consisting of EPcIA-001 to EPcIA-142 of Table a or a sequence having at least 50%, 60%, 70%, 80%, 90%, 91%, 92%, 93%, 94%, 95%, 96%, 97%, 98% or 99% sequence identity therewith, or a fragment or derivative thereof, in combination one or more sequences independently selected from the group consisting of EPcIB-001 to EPcIB-021 of Table B, or a sequence having at least 50%, 60%, 70%, 80%, 90%, 91%, 92%, 93%, 94%, 95%, 96%, 97%, 98% or 99% sequence identity therewith, or a fragment or derivative thereof; and optionally in combination with one or more sequences independently selected from the group consisting of EPcIC-001 to EPcIC-005 of Table C, or a sequence having at least 50%, 60%, 70%, 80%, 90%, 91%, 92%, 93%, 94%, 95%, 96%, 97%, 98% or 99% sequence identity therewith, or a fragment or derivative thereof and/or EPcID-001 of Table D, or a sequence having at least 50%, 60%, 70%, 80%, 90%, 91%, 92%, 93%, 94%, 95%, 96%, 97%, 98% or 99% sequence identity therewith, or a fragment or derivative thereof;
ii) one or more sequences independently selected from the group consisting of EPcIA-001 to EPcIA-142 of Table A or a sequence having at least 50%, 60%, 70%, 80%, 90%, 91%, 92%, 93%, 94%, 95%, 96%, 97%, 98% or 99% sequence identity therewith, or a fragment or derivative thereof, in combination one or more sequences independently selected from the group consisting of EPcIC-001 to EPcIC-005 of Table C, or a sequence having at least 50%, 60%, 70%, 80%, 90%, 91%, 92%, 93%, 94%, 95%, 96%, 97%, 98% or 99% sequence identity therewith, or a fragment or derivative thereof; and optionally in combination with one or more sequences independently selected from the group consisting of EPcIB-001 to EPcIB-021 of Table B, or a sequence having at least 50%, 60%, 70%, 80%, 90%, 91%, 92%, 93%, 94%, 95%, 96%, 97%, 98% or 99% sequence identity therewith, or a fragment or derivative thereof and/or EPcID-001 of Table D or a sequence having at least 50%, 60%, 70%, 80%, 90%, 91%, 92%, 93%, 94%, 95%, 96%, 97%, 98% or 99% sequence identity therewith, or a fragment or derivative thereof;
iii) one or more sequences independently selected from the group consisting of EPcIA-001 to EPcIA-142 of Table A or a sequence having at least 50%, 60%, 70%, 80%, 90%, 91%, 92%, 93%, 94%, 95%, 96%, 97%, 98% or 99% sequence identity therewith, or a fragment or derivative thereof, in combination and EPcID-001 of Table D, or a sequence having at least 50%, 60%, 70%, 80%, 90%, 91%, 92%, 93%, 94%, 95%, 96%, 97%, 98% or 99% sequence identity therewith, or a fragment or derivative thereof, and optionally or a sequence having at least 50%, 60%, 70%, 80%, 90%, 91%, 92%, 93%, 94%, 95%, 96%, 97%, 98% or 99% sequence identity therewith, or a fragment or derivative thereof one or more sequences independently selected from the group consisting of EPcIB-001 to EPcIB-021 of Table B, or a sequence having at least 50%, 60%, 70%, 80%, 90%, 91%, 92%, 93%, 94%, 95%, 96%, 97%, 98% or 99% sequence identity therewith, or a fragment or derivative thereof and/or EPcIC-001 to EPcIC-005 of Table C, or a sequence having at least 50%, 60%, 70%, 80%, 90%, 91%, 92%, 93%, 94%, 95%, 96%, 97%, 98% or 99% sequence identity therewith, or a fragment or derivative thereof;
iv) one or more sequences independently selected from the group consisting of EPcIB-001 to EPcIB-021 of Table B, or a sequence having at least 50%, 60%, 70%, 80%, 90%, 91%, 92%, 93%, 94%, 95%, 96%, 97%, 98% or 99% sequence identity therewith, or a fragment or derivative thereof, in combination one or more sequences independently selected from the group consisting of EPcIC-001 to EPcIC-005 of Table C, or a sequence having at least 50%, 60%, 70%, 80%, 90%, 91%, 92%, 93%, 94%, 95%, 96%, 97%, 98% or 99% sequence identity therewith, or a fragment or derivative thereof; and optionally or a sequence having at least 50%, 60%, 70%, 80%, 90%, 91%, 92%, 93%, 94%, 950/0, 96%, 97%, 98% or 99% sequence identity therewith, or a fragment or derivative thereof one or more sequences independently selected from the group consisting of EPcIA-001 to EPcIA-142 of Table A or a sequence having at least 50%, 60%, 70%, 80%, 90%, 91%, 92%, 93%, 94%, 95%, 96%, 97%, 98% or 99% sequence identity therewith, or a fragment or derivative thereof and/or EPcID-001 of Table D, or a sequence having at least 50%, 60%, 70%, 80%, 90%, 91%, 92%, 93%, 94%, 95%, 96%, 97%, 98% or 99% sequence identity therewith, or a fragment or derivative thereof;
v) one or more sequences independently selected from the group consisting of EPcIC-001 to EPcIC-005 of Table C, or a sequence having at least 50%, 60%, 70%, 80%, 90%, 91%, 92%, 93%, 94%, 95%, 96%, 97%, 98% or 99% sequence identity therewith, or a fragment or derivative thereof, in combination with EPcID-001 of Table D or a sequence having at least 50%, 60%, 70%, 80%, 90%, 91%, 92%, 93%, 94%, 95%, 96%, 97%, 98% or 99% sequence identity therewith, or a fragment or derivative thereof; and optionally in combination with one or more sequences independently selected from the group consisting of EPcIA-001 to EPcIA-142 of Table A, or a sequence having at least 50%, 60%, 70%, 80%, 90%, 91%, 92%, 93%, 94%, 95%, 96%, 97%, 98% or 99% sequence identity therewith, or a fragment or derivative thereof; and/or one or more sequences independently selected from the group consisting of EPcIB-001 to EPcIB-021 of Table B or a sequence having at least 50%, 60%, 70%, 80%, 90%, 91%, 92%, 93%, 94%, 95%, 96%, 97%, 98% or 99% sequence identity therewith, or a fragment or derivative thereof; and
vi) one or more sequences independently selected from the group consisting of EPcIB-001 to EPcIB-021 of Table B or a sequence having at least 50%, 60%, 70%, 80%, 90%, 91%, 92%, 93%, 94%, 95%, 96%, 97%, 98% or 99% sequence identity therewith, or a fragment thereof, in combination with EPcID-001 of Table D or a sequence having at least 50%, 60%, 70%, 80%, 90%, 91%, 92%, 93%, 94%, 95%, 96%, 97%, 98% or 99% sequence identity therewith, or a fragment or derivative thereof; optionally in combination with of EPcIC-001 to EPcIC-005 of Table C, or a sequence having at least 50%, 60%, 70%, 80%, 90%, 91%, 92%, 93%, 94%, 95%, 96%, 97%, 98% or 99% sequence identity therewith, or a fragment or derivative thereof and/or EPcIA-001 to EPcIA-142 of Table A, or a sequence having at least 50%, 60%, 70%, 80%, 90%, 91%, 92%, 93%, 94%, 95%, 96%, 97%, 98% or 99% sequence identity therewith, or a fragment or derivative thereof.

19. A fusion protein according to claim 1, wherein two or more PVTD's are provided, and the combination of PVTD's is selected from:
i) one or more sequences independently selected from the group consisting of PfN-01 to PfN-86 of Table H or a sequence having at least 50%, 60%, 70%, 80%, 90%, 91%, 92%, 93%, 94%, 95%, 96%, 97%, 98% or 99% sequence identity therewith, or a fragment or derivative thereof, in combination one or more sequences independently selected from the group consisting of PCoil-01 to PCoil-46 of Table I, or a sequence having at least 50%, 60%, 70%, 80%, 90%, 91%, 92%, 93%, 94%, 95%, 96%, 97%, 98% or 99% sequence identity therewith, or a fragment or derivative thereof and optionally in combination with one or more sequences independently selected from the group consisting of PfC-01 to PfC-61 of Table J, or a sequence having at least 50%, 60%, 70%, 80%, 90%, 91%, 92%, 93%, 94%, 95%, 96%, 97%, 98% or 99% sequence identity therewith, or a fragment or derivative thereof and/or a Pf2 sequence preferably from one of the Pf2 domains in sequences EPcIB-001 to EPcIB-021 of Table B, or a sequence having at least 50%, 60%, 70%, 80%, 90%, 91%, 92%, 93%, 94%, 95%, 96%, 97%, 98% or 99% sequence identity therewith, or a fragment or derivative thereof;
ii) one or more sequences independently selected from the group consisting of PfN-01 to PfN-86 of Table H or a sequence having at least 50%, 60%, 70%, 80%, 90%, 91%, 92%, 93%, 94%, 95%, 96%, 97%, 98% or 99% sequence identity therewith, or a fragment or derivative thereof, in combination one or more sequences independently selected from the group consisting of PfC-01 to PfC-61 of Table J, or a sequence having at least 50%, 60%, 70%, 80%, 90%, 91%, 92%, 93%, 94%, 95%, 96%, 97%, 98% or 99% sequence identity therewith, or a fragment or derivative thereof and optionally in combination with one or more sequences independently selected from the group consisting of PCoil-01 to PCoil-46 of Table I, or a sequence having at least 50%, 60%, 70%, 80%, 90%, 91%, 92%, 93%, 94%, 95%, 96%, 97%, 98% or 99% sequence identity therewith, or a fragment or derivative thereof and/or a Pf2 sequence, preferably from one of the Pf2 domains in sequences EPcIB-001 to EPcIB-021 of Table B, or a sequence having at least 50%, 60%, 70%, 80%, 90%, 91%, 92%, 93%, 94%, 95%, 96%, 97%, 98% or 99% sequence identity therewith, or a fragment or derivative thereof;
iii) one or more sequences independently selected from the group consisting of PfN-01 to PfN-86 of Table H or a sequence having at least 50%, 60%, 70%, 80%, 90%, 91%, 92%, 93%, 94%, 95%, 96%, 97%, 98% or 99% sequence identity therewith, or a fragment or derivative thereof, in combination with a Pf2 sequence, preferably from one of the Pf2 domains in sequences EPcIB-001 to EPcIB-021 of Table B, or a sequence having at least 50%, 60%, 70%, 80%, 90%, 91%, 92%, 93%, 94%, 95%, 96%, 97%, 98% or 99% sequence identity therewith, or a fragment or derivative thereof, and optionally in combination with one or more sequences independently selected from the group consisting of PfC-01 to PfC-61 of Table J, or a sequence having at least 50%, 60%, 70%, 80%, 90%, 91%, 92%, 93%, 94%, 95%, 96%, 97%, 98% or 99% sequence identity therewith, or a fragment or derivative thereof and/or PCoil-01 to PCoil-46 of Table I, or a sequence having at least 50%, 60%, 70%, 80%, 90%, 91%, 92%, 93%, 94%, 95%, 96%, 97%, 98% or 99% sequence identity therewith, or a fragment or derivative thereof;
iv) one or more sequences independently selected from the group consisting of PCoil-01 to PCoil-46 of Table I, or a sequence having at least 50%, 60%, 70%, 80%, 90%, 91%, 92%, 93%, 94%, 95%, 96%, 97%, 98% or 99% sequence identity therewith, or a fragment or derivative thereof, in combination one or more sequences independently selected from the group consisting of PfC-01 to PfC-61 of Table J, or a sequence having at least 50%, 60%, 70%, 80%, 90%, 91%, 92%, 93%, 94%, 95%, 96%, 97%, 98% or 99% sequence identity therewith, or a fragment or derivative thereof; and optionally in combination with one or more sequences independently selected from the group consisting of PfN-01 to PfN-86 of Table H or a sequence having at least 50%, 60%, 70%, 80%, 90%, 91%, 92%, 93%, 94%, 95%, 96%, 97%, 98% or 99% sequence identity therewith, or a fragment or derivative thereof and/or a Pf2 sequence, preferably from one of the Pf2 domains in sequences EPcIB-001 to EPcIB-021 of Table B, or a sequence having at least 50%, 60%, 70%, 80%, 90%, 91%, 92%, 93%, 94%, 95%, 96%, 97%, 98% or 99% sequence identity therewith, or a fragment or derivative thereof;
v) one or more sequences independently selected from the group consisting of PCoil-01 to PCoil-46 of Table I, or a sequence having at least 50%, 60%, 70%, 80%, 90%, 91%, 92%, 93%, 94%, 95%, 96%, 97%, 98% or 99% sequence identity therewith, or a fragment or derivative thereof, in combination with a Pf2 sequence, preferably from one of the Pf2 domains in sequences EPcIB-001 to EPcIB-021 of Table B, or a sequence having at least 50%, 60%, 70%, 80%, 90%, 91%, 92%, 93%, 94%, 95%, 96%, 97%, 98% or 99% sequence identity therewith, or a fragment or derivative thereof; and optionally in combination with one or more sequences independently selected from the group consisting of PfN-01 to PfN-86 of Table H, or a sequence having at least 50%, 60%, 70%, 80%, 90%, 91%, 92%, 93%, 94%, 95%, 96%, 97%, 98% or 99% sequence identity therewith, or a fragment or derivative thereof; and/or one or more sequences independently selected from the group consisting of PfC-01 to PfC-61 of Table J or a sequence having at least 50%, 60%, 70%, 80%, 90%, 91%, 92%, 93%, 94%, 95%, 96%, 97%, 98% or 99% sequence identity therewith, or a fragment or derivative thereof; and
vi) one or more sequences independently selected from the group consisting of PfC-01 to PfC-61 of Table J, or a sequence having at least 50%, 60%, 70%, 80%, 90%, 91%, 92%, 93%, 94%, 95%, 96%, 97%, 98% or 99% sequence identity therewith, or a fragment or derivative thereof, in combination with a Pf2 sequence, preferably from one of the Pf2 domains in sequences EPcIB-001 to EPcIB-021 of Table B, or a sequence having at least 50%, 60%, 70%, 80%, 90%, 91%, 92%, 93%, 94%, 95%, 96%, 97%, 98% or 99% sequence identity therewith, or a fragment or derivative thereof; and optionally in combination with one or more sequences independently selected from the group consisting of PfN-01 to PfN-86 of Table H, or a sequence having at least 50%, 60%, 70%, 80%, 90%, 91%, 92%, 93%, 94%, 95%, 96%, 97%, 98% or 99% sequence identity therewith, or a fragment or derivative thereof; and/or one or more sequences independently selected from the group consisting of PCoil-01 to PCoil-46 of Table I or a sequence having at least 50%, 60%, 70%, 80%, 90%, 91%, 92%, 93%, 94%, 95%, 96%, 97%, 98% or 99% sequence identity therewith, or a fragment or derivative thereof.

20. A nucleic acid sequence encoding a trimeric fusion protein comprising three polypeptide chains, wherein each polypeptide chain comprises a eukaryotic collagen or collagen-like domain and a PVTD.

21. A nucleic acid sequence encoding a fusion protein, as defined in claim 1.

22. A vector comprising a nucleic acid sequence according to claim 20.

23. A vector according to claim 22, wherein the vector is an expression vector.

24. A host cell comprising a fusion protein according to claim 1.

25. A method of producing a trimeric fusion protein comprising three polypeptide chains, wherein each polypeptide chain comprises a eukaryotic collagen or collagen-like domain and a PVTD, the method comprising:
i) introducing into a host cell one or more nucleic acid sequences encoding a fusion protein or polypeptide of the invention;
ii) culturing the host cell under conditions suitable for expression of said fusion protein or fusion polypeptide and formation of a trimeric fusion protein comprising three of said polypeptide chains; and
iii) optionally isolating the expressed fusion protein from the host cell, preferably wherein the fusion protein is as defined in claim 1.

26. (canceled)

27. A method of producing a fusion protein comprising three polypeptide chains, wherein each polypeptide chain comprises a eukaryotic collagen or collagen-like domain and a PVTD in a cell free system, the method comprising:
i) introducing into a cell-free expression system one or more nucleic acid sequences encoding said fusion protein polypeptide;
ii) maintaining the cell-free expression system under conditions suitable for expression of said fusion protein or fusion polypeptide and formation of a trimeric fusion protein comprising three of said polypeptide chains; and
iii) optionally isolating the expressed fusion protein from the expression system, preferably wherein the fusion protein is as defined in claim 1.

28. A method of producing a fusion polypeptide comprising a eukaryotic collagen or collagen-like domain and a PVTD, the method comprising:
i) introducing into a cell-free expression system a nucleic acid sequence encoding said fusion polypeptide of the invention;
ii) maintaining the cell-free expression system under conditions suitable for expression of said fusion polypeptide; and
iii) optionally isolating the expressed fusion polypeptide from the host cell, preferably wherein the fusion polypeptide is as defined in claim 5.

29. A method of producing a gelatine-like protein, comprising:
i) introducing into a host cell one or more nucleic acid sequences encoding said fusion protein;
ii) culturing the host cell under conditions suitable for expression and formation of a trimeric fusion protein comprising three of said polypeptide chains;
iii) optionally isolating the expressed fusion protein from the host cell, wherein the fusion protein is as defined in claim 1; and
iv) fully or partially denaturing and/or fragmenting the trimeric fusion protein of iii) to produce a gelatine-like protein.

30. A method of producing a gelatine-like protein, in a cell free system, the method comprising:
i) introducing into a cell-free expression system one or more nucleic acid sequences encoding said fusion protein;
ii) maintaining the cell-free expression system under conditions suitable for expression and formation of a trimeric fusion protein comprising three of said polypeptide chains;
iii) optionally isolating the expressed fusion protein from the expression system, wherein the fusion protein is as defined in claim 1, and
iv) fully or partially denaturing and/or fragmenting a trimeric fusion protein of iii) to produce a gelatine-like protein.

31. A method of producing a fusion protein according to claim 25, further comprising purifying the fusion protein.

32. A product comprising a fusion protein as defined in claim 1.

33. A product according to claim 32, selected from the group consisting of a foodstuff, cosmetic, stabilizer, capsules, biomaterial, medical device, medicament, artificial tissue, pharmaceutical or nutritional supplement, chemical or biochemical reagent, or glue.

34. A fusion protein as defined in claim 1, for use in the treatment or prevention of a collagen-related disorder.

35. A method of treatment or prevention of a collagen-related disorder, comprising administrating to a subject a fusion protein as defined in claim 1.

36. Use of a fusion protein as defined in claim 1, in the manufacture of a product.

37. Use according to claim 36, wherein the product is selected from the group consisting of a foodstuff, cosmetic, stabilizer, capsules, biomaterial, medical device, medicament, artificial tissue, pharmaceutical or nutritional supplement, chemical or biochemical reagent, or glue.

38. A fusion polypeptide according to claim 5, wherein the PVTD is derived from a collagen or collagen-like protein.

39. A fusion polypeptide according to claim 5, wherein the PVTD may be provided:
i) within a eukaryotic collagen or collagen-like domain; and/or
ii) flanking one or both ends of a eukaryotic collagen or collagen-like domain; and/or
iii) within non-eukaryotic collagen or collagen-like domain of the fusion polypeptide and/or flanking one or both ends thereof.

40. A fusion polypeptide according to claim 5, wherein the PVTD comprises one or more functional sequences independently selected from the group consisting of stabilization sequences, binding sites, cleavage sites, and linkage sites.

41. A fusion polypeptide according to claim 5, wherein the eukaryotic collagen or collagen-like domain is derived from vertebrate collagen or collagen-like proteins, preferably mammalian, ruminate, fish, or preferably human.

42. A fusion polypeptide according to claim 5 wherein the eukaryotic collagen or collagen-like domain of the fusion protein or polypeptide is composed of two or more heterologous collagen or collagen-like domains operably linked to form a single collagen or collagen-like domain.

43. A fusion polypeptide according to claim 42, wherein more than one eukaryotic collagen or collagen-like domains is present, and wherein one or more or all may be chimeric.

44. A fusion polypeptide according to claim 5, wherein the eukaryotic collagen or collagen-like domain comprises:
i) a human fibrillar collagen chain selected from a1(I), 2(I), a1(II) and a1(III);
ii) a eukaryotic collagen or collagen-like domain comprising a sequence selected from the group consisting of sequences hCol-01 to hCol-89 of Tables K and L;
iii) a sequence consisting of a sequence selected from the groups consisting of the human collagen sequences any of hCol-01 to hCol-49 of Table K and the collagen-like domains of any of hCol-50 to hCol-89 of Table L;
iv) a domain or sequence having at least 50%, 60%, 70%, 80%, 90%, 91%, 92%, 93%, 94%, 95%, 96%, 97%, 98% or 99% sequence identity with a sequence of i), ii) or iii); or
v) fragments, variants or derivatives of a sequence of any of i) to iv).

45. A fusion polypeptide according to claim 5, comprising one or more THDs (triple helical domains), either in tandem or separated by one or more PVTDs or other sequences.

46. A fusion polypeptide according to claim 5, further comprising one or more functional domains, selected from the group consisting of binding sites, cleavage sites, linkage sites, and trimerisation sites.

47. A fusion polypeptide according to claim 5 wherein a eukaryotic collagen or collagen-like domain may be independently selected from the group consisting of vertebrate, mammalian, ruminate, fish, or human collagen or collagen-like proteins.

48. A fusion polypeptide according to claim 5, wherein the PVTD is derived from a bacterial source, preferably gram negative bacteria, preferably pathogenic E. coli, preferably E. coli strain O157:H7.

49. A fusion polypeptide according to claim 5, wherein the PVTD may be:
i) a PVTD of any of EPcIA-001 to EPcIA-142 of Table A, any of EPcIB-001 to EPcIB-021 of Table B, any of EPcIC-001 to EPcIC-005 of Table C, or EPcID-001 of Table D, any of PfN-01 to PfN-86 of Table H, any of PCoil-01 to PCoil-46 of Table I, any of PfC-01 to PfC-61 of Table J, and a Pf2 sequence, preferably one of the Pf2 domains in sequences any of EPcIB-001 to EPcIB-021 of Table B;
ii) having an amino acid sequence having at least 50%, 60%, 70%, 80%, 90%, 91%, 92%, 93%, 94%, 95%, 96%, 97%, 98% or 99% sequence identity with a PVTD of i);
iii) encoded by a nucleic acid selected from the group consisting of sequences of Table E to G and M to R or a nucleic acid sequence having at least 50%, 60%, 70%, 80%, 90%, 91%, 92%, 93%, 94%, 95%, 96%, 97%, 98% or 99% sequence thereto; or
iv) a fragment or derivative of an afore-mentioned sequence which functions as a PVTD

50. A fusion polypeptide according to claim 5, wherein the fusion polypeptide comprises two or more PVTDs, the combination of PVTD's being selected from:
i) one or more sequences independently selected from the group consisting of EPcIA-001 to EPcIA-142 of Table a or a sequence having at least 50%, 60%, 70%, 80%, 90%, 91%, 92%, 93%, 94%, 95%, 96%, 97%, 98% or 99% sequence identity therewith, or a fragment or derivative thereof, in combination one or more sequences independently selected from the group consisting of EPcIB-001 to EPcIB-021 of Table B, or a sequence having at least 50%, 60%, 70%, 80%, 90%, 91%, 92%, 93%, 94%, 95%, 96%, 97%, 98% or 99% sequence identity therewith, or a fragment or derivative thereof; and optionally in combination with one or more sequences independently selected from the group consisting of EPcIC-001 to EPcIC-005 of Table C, or a sequence having at least 50%, 60%, 70%, 80%, 90%, 91%, 92%, 93%, 94%, 95%, 96%, 97%, 98% or 99% sequence identity therewith, or a fragment or derivative thereof and/or EPcID-001 of Table D, or a sequence having at least 50%, 60%, 70%, 80%, 90%, 91%, 92%, 93%, 94%, 95%, 96%, 97%, 98% or 99% sequence identity therewith, or a fragment or derivative thereof;
ii) one or more sequences independently selected from the group consisting of EPcIA-001 to EPcIA-142 of Table A or a sequence having at least 50%, 60%, 70%, 80%, 90%, 91%, 92%, 93%, 94%, 95%, 96%, 97%, 98% or 99% sequence identity therewith, or a fragment or derivative thereof, in combination one or more sequences independently selected from the group consisting of EPcIC-001 to EPcIC-005 of Table C, or a sequence having at least 50%, 60%, 70%, 80%, 90%, 91%, 92%, 93%, 94%, 95%, 96%, 97%, 98% or 99% sequence identity therewith, or a fragment or derivative thereof; and optionally in combination with one or more sequences independently selected from the group consisting of EPcIB-001 to EPcIB-021 of Table B, or a sequence having at least 50%, 60%, 70%, 80%, 90%, 91%, 92%, 93%, 94%, 95%, 96%, 97%, 98% or 99% sequence identity therewith, or a fragment or derivative thereof and/or EPcID-001 of Table D or a sequence having at least 50%, 60%, 70%, 80%, 90%, 91%, 92%, 93%, 94%, 95%, 96%, 97%, 98% or 99% sequence identity therewith, or a fragment or derivative thereof;
iii) one or more sequences independently selected from the group consisting of EPcIA-001 to EPcIA-142 of Table A or a sequence having at least 50%, 60%, 70%, 80%, 90%, 91%, 92%, 93%, 94%, 95%, 96%, 97%, 98% or 99% sequence identity therewith, or a fragment or derivative thereof, in combination and EPcID-001 of Table D, or a sequence having at least 50%, 60%, 70%, 80%, 90%, 91%, 92%, 93%, 94%, 95%, 96%, 97%, 98% or 99% sequence identity therewith, or a fragment or derivative thereof, and optionally or a sequence having at least 50%, 60%, 70%, 80%, 90%, 91%, 92%, 93%, 94%, 95%, 96%, 97%, 98% or 99% sequence identity therewith, or a fragment or derivative thereof one or more sequences independently selected from the group consisting of EPcIB-001 to EPcIB-021 of Table B, or a sequence having at least 50%, 60%, 70%, 80%, 90%, 91%, 92%, 93%, 94%, 95%, 96%, 97%, 98% or 99% sequence identity therewith, or a fragment or derivative thereof and/or EPcIC-001 to EPcIC-005 of Table C, or a sequence having at least 50%, 60%, 70%, 80%, 90%, 91%, 92%, 93%, 94%, 95%, 96%, 97%, 98% or 99% sequence identity therewith, or a fragment or derivative thereof;
iv) one or more sequences independently selected from the group consisting of EPcIB-001 to EPcIB-021 of Table B, or a sequence having at least 50%, 60%, 70%, 80%, 90%, 91%, 92%, 93%, 94%, 95%, 96%, 97%, 98% or 99% sequence identity therewith, or a fragment or derivative thereof, in combination one or more sequences independently selected from the group consisting of EPcIC-001 to EPcIC-005 of Table C, or a sequence having at least 50%, 60%, 70%, 80%, 90%, 91%, 92%, 93%, 94%, 95%, 96%, 97%, 98% or 99% sequence identity therewith, or a fragment or derivative thereof; and optionally or a sequence having at least 50%, 60%, 70%, 80%, 90%, 91%, 92%, 93%, 94%, 950/0, 96%, 97%, 98% or 99% sequence identity therewith, or a fragment or derivative thereof one or more sequences independently selected from the group consisting of EPcIA-001 to EPcIA-142 of Table A or a sequence having at least 50%, 60%, 70%, 80%, 90%, 91%, 92%, 93%, 94%, 95%, 96%, 97%, 98% or 99% sequence identity therewith, or a fragment or derivative thereof and/or EPcID-001 of Table D, or a sequence having at least 50%, 60%, 70%, 80%, 90%, 91%, 92%, 93%, 94%, 95%, 96%, 97%, 98% or 99% sequence identity therewith, or a fragment or derivative thereof;
v) one or more sequences independently selected from the group consisting of EPcIC-001 to EPcIC-005 of Table C, or a sequence having at least 50%, 60%, 70%, 80%, 90%, 91%, 92%, 93%, 94%, 95%, 96%, 97%, 98% or 99% sequence identity therewith, or a fragment or derivative thereof, in combination with EPcID-001 of Table D or a sequence having at least 50%, 60%, 70%, 80%, 90%, 91%, 92%, 93%, 94%, 95%, 96%, 97%, 98% or 99% sequence identity therewith, or a fragment or derivative thereof; and optionally in combination with one or more sequences independently selected from the group consisting of EPcIA-001 to EPcIA-142 of Table A, or a sequence having at least 50%, 60%, 70%, 80%, 90%, 91%, 92%, 93%, 94%, 95%, 96%, 97%, 98% or 99% sequence identity therewith, or a fragment or derivative thereof; and/or one or more sequences independently selected from the group consisting of EPcIB-001 to EPcIB-021 of Table B or a sequence having at least 50%, 60%, 70%, 80%, 90%, 91%, 92%, 93%, 94%, 95%, 96%, 97%, 98% or 99% sequence identity therewith, or a fragment or derivative thereof; and
vi) one or more sequences independently selected from the group consisting of EPcIB-001 to EPcIB-021 of Table B or a sequence having at least 50%, 60%, 70%, 80%, 90%, 91%, 92%, 93%, 94%, 95%, 96%, 97%, 98% or 99% sequence identity therewith, or a fragment thereof, in combination with EPcID-001 of Table D or a sequence having at least 50%, 60%, 70%, 80%, 90%, 91%, 92%, 93%, 94%, 95%, 96%, 97%, 98% or 99% sequence identity therewith, or a fragment or derivative thereof; optionally in combination with of EPcIC-001 to EPcIC-005 of Table C, or a sequence having at least 50%, 60%, 70%, 80%, 90%, 91%, 92%, 93%, 94%, 95%, 96%, 97%, 98% or 99% sequence identity therewith, or a fragment or derivative thereof and/or EPcIA-001 to EPcIA-142 of Table A, or a sequence having at least 50%, 60%, 70%, 80%, 90%, 91%, 92%, 93%, 94%, 95%, 96%, 97%, 98% or 99% sequence identity therewith, or a fragment or derivative thereof.

51. A fusion polypeptide according to claim 5, wherein two or more PVTD's are provided, and the combination of PVTD's is selected from:
i) one or more sequences independently selected from the group consisting of PfN-01 to PfN-86 of Table H or a sequence having at least 50%, 60%, 70%, 80%, 90%, 91%, 92%, 93%, 94%, 95%, 96%, 97%, 98% or 99% sequence identity therewith, or a fragment or derivative thereof, in combination one or more sequences independently selected from the group consisting of PCoil-01 to PCoil-46 of Table I, or a sequence having at least 50%, 60%, 70%, 80%, 90%, 91%, 92%, 93%, 94%, 95%, 96%, 97%, 98% or 99% sequence identity therewith, or a fragment or derivative thereof and optionally in combination with one or more sequences independently selected from the group consisting of PfC-01 to PfC-61 of Table J, or a sequence having at least 50%, 60%, 70%, 80%, 90%, 91%, 92%, 93%, 94%, 95%, 96%, 97%, 98% or 99% sequence identity therewith, or a fragment or derivative thereof and/or a Pf2 sequence preferably from one of the Pf2 domains in sequences EPcIB-001 to EPcIB-021 of Table B, or a sequence having at least 50%, 60%, 70%, 80%, 90%, 91%, 92%, 93%, 94%, 95%, 96%, 97%, 98% or 99% sequence identity therewith, or a fragment or derivative thereof;
ii) one or more sequences independently selected from the group consisting of PfN-01 to PfN-86 of Table H or a sequence having at least 50%, 60%, 70%, 80%, 90%, 91%, 92%, 93%, 94%, 95%, 96%, 97%, 98% or 99% sequence identity therewith, or a fragment or derivative thereof, in combination one or more sequences independently selected from the group consisting of PfC-01 to PfC-61 of Table J, or a sequence having at least 50%, 60%, 70%, 80%, 90%, 91%, 92%, 93%, 94%, 95%, 96%, 97%, 98% or 99% sequence identity therewith, or a fragment or derivative thereof and optionally in combination with one or more sequences independently selected from the group consisting of PCoil-01 to PCoil-46 of Table I, or a sequence having at least 50%, 60%, 70%, 80%, 90%, 91%, 92%, 93%, 94%, 95%, 96%, 97%, 98% or 99% sequence identity therewith, or a fragment or derivative thereof and/or a Pf2 sequence, preferably from one of the Pf2 domains in sequences EPcIB-001 to EPcIB-021 of Table B, or a sequence having at least 50%, 60%, 70%, 80%, 90%, 91%, 92%, 93%, 94%, 95%, 96%, 97%, 98% or 99% sequence identity therewith, or a fragment or derivative thereof;
iii) one or more sequences independently selected from the group consisting of PfN-01 to PfN-86 of Table H or a sequence having at least 50%, 60%, 70%, 80%, 90%, 91%, 92%, 93%, 94%, 95%, 96%, 97%, 98% or 99% sequence identity therewith, or a fragment or derivative thereof, in combination with a Pf2 sequence, preferably from one of the Pf2 domains in sequences EPcIB-001 to EPcIB-021 of Table B, or a sequence having at least 50%, 60%, 70%, 80%, 90%, 91%, 92%, 93%, 94%, 95%, 96%, 97%, 98% or 99% sequence identity therewith, or a fragment or derivative thereof, and optionally in combination with one or more sequences independently selected from the group consisting of PfC-01 to PfC-61 of Table J, or a sequence having at least 50%, 60%, 70%, 80%, 90%, 91%, 92%, 93%, 94%, 95%, 96%, 97%, 98% or 99% sequence identity therewith, or a fragment or derivative thereof and/or PCoil-01 to PCoil-46 of Table I, or a sequence having at least 50%, 60%, 70%, 80%, 90%, 91%, 92%, 93%, 94%, 95%, 96%, 97%, 98% or 99% sequence identity therewith, or a fragment or derivative thereof;
iv) one or more sequences independently selected from the group consisting of PCoil-01 to PCoil-46 of Table I, or a sequence having at least 50%, 60%, 70%, 80%, 90%, 91%, 92%, 93%, 94%, 95%, 96%, 97%, 98% or 99% sequence identity therewith, or a fragment or derivative thereof, in combination one or more sequences independently selected from the group consisting of PfC-01 to PfC-61 of Table J, or a sequence having at least 50%, 60%, 70%, 80%, 90%, 91%, 92%, 93%, 94%, 95%, 96%, 97%, 98% or 99% sequence identity therewith, or a fragment or derivative thereof; and optionally in combination with one or more sequences independently selected from the group consisting of PfN-01 to PfN-86 of Table H or a sequence having at least 50%, 60%, 70%, 80%, 90%, 91%, 92%, 93%, 94%, 95%, 96%, 97%, 98% or 99% sequence identity therewith, or a fragment or derivative thereof and/or a Pf2 sequence, preferably from one of the Pf2 domains in sequences EPcIB-001 to EPcIB-021 of Table B, or a sequence having at least 50%, 60%, 70%, 80%, 90%, 91%, 92%, 93%, 94%, 95%, 96%, 97%, 98% or 99% sequence identity therewith, or a fragment or derivative thereof;
v) one or more sequences independently selected from the group consisting of PCoil-01 to PCoil-46 of Table I, or a sequence having at least 50%, 60%, 70%, 80%, 90%, 91%, 92%, 93%, 94%, 95%, 96%, 97%, 98% or 99% sequence identity therewith, or a fragment or derivative thereof, in combination with a Pf2 sequence, preferably from one of the Pf2 domains in sequences EPcIB-001 to EPcIB-021 of Table B, or a sequence having at least 50%, 60%, 70%, 80%, 90%, 91%, 92%, 93%, 94%, 95%, 96%, 97%, 98% or 99% sequence identity therewith, or a fragment or derivative thereof; and optionally in combination with one or more sequences independently selected from the group consisting of PfN-01 to PfN-86 of Table H, or a sequence having at least 50%, 60%, 70%, 80%, 90%, 91%, 92%, 93%, 94%, 95%, 96%, 97%, 98% or 99% sequence identity therewith, or a fragment or derivative thereof; and/or one or more sequences independently selected from the group consisting of PfC-01 to PfC-61 of Table J or a sequence having at least 50%, 60%, 70%, 80%, 90%, 91%, 92%, 93%, 94%, 95%, 96%, 97%, 98% or 99% sequence identity therewith, or a fragment or derivative thereof; and
vi) one or more sequences independently selected from the group consisting of PfC-01 to PfC-61 of Table J, or a sequence having at least 50%, 60%, 70%, 80%, 90%, 91%, 92%, 93%, 94%, 95%, 96%, 97%, 98% or 99% sequence identity therewith, or a fragment or derivative thereof, in combination with a Pf2 sequence, preferably from one of the Pf2 domains in sequences EPcIB-001 to EPcIB-021 of Table B, or a sequence having at least 50%, 60%, 70%, 80%, 90%, 91%, 92%, 93%, 94%, 95%, 96%, 97%, 98% or 99% sequence identity therewith, or a fragment or derivative thereof; and optionally in combination with one or more sequences independently selected from the group consisting of PfN-01 to PfN-86 of Table H, or a sequence having at least 50%, 60%, 70%, 80%, 90%, 91%, 92%, 93%, 94%, 95%, 96%, 97%, 98% or 99% sequence identity therewith, or a fragment or derivative thereof; and/or one or more sequences independently selected from the group consisting of PCoil-01 to PCoil-46 of Table I or a sequence having at least 50%, 60%, 70%, 80%, 90%, 91%, 92%, 93%, 94%, 95%, 96%, 97%, 98% or 99% sequence identity therewith, or a fragment or derivative thereof.

52. A nucleic acid sequence encoding a fusion polypeptide, as defined in claim 5.

53. A vector comprising a nucleic acid sequence according to claim 52.

54. A vector according to claim 53, wherein the vector is an expression vector.

55. A host cell comprising a fusion polypeptide according to claim 5.

56. A method of producing a fusion polypeptide comprising a eukaryotic collagen or collagen-like domain and a PVTD, the method comprising:
i) introducing into a host cell a nucleic acid sequence encoding said fusion polypeptide of the invention;
ii) culturing the host cell under conditions suitable for expression of said fusion polypeptide; and
iii) optionally isolating the expressed fusion polypeptide from the host cell, preferably wherein the fusion polypeptide is as defined in claim 38.

57. A method of producing a fusion polypeptide comprising a eukaryotic collagen or collagen-like domain and a PVTD, the method comprising:
i) introducing into a cell-free expression system a nucleic acid sequence encoding said fusion polypeptide of the invention;
ii) maintaining the cell-free expression system under conditions suitable for expression of said fusion polypeptide; and
iii) optionally isolating the expressed fusion polypeptide from the host cell, preferably wherein the fusion polypeptide is as defined in claim 5.

58. A method of producing a fusion polypeptide according to claim 56, further comprising purifying the fusion polypeptide.

59. A product comprising a fusion polypeptide as defined in claim 5.

60. A product according to claim 59, selected from the group consisting of a foodstuff, cosmetic, stabilizer, capsules, biomaterial, medical device, medicament, artificial tissue, pharmaceutical or nutritional supplement, chemical or biochemical reagent, or glue.

61. A fusion polypeptide as defined in claim 5, for use in the treatment or prevention of a collagen-related disorder.

62. A method of treatment or prevention of a collagen-related disorder, comprising administrating to a subject a fusion polypeptide as defined in claim 5.

63. Use of a fusion polypeptide as defined in claim 5, in the manufacture of a product.

64. Use according to claim 63, wherein the product is selected from the group consisting of a foodstuff, cosmetic, stabilizer, capsules, biomaterial, medical device, medicament, artificial tissue, pharmaceutical or nutritional supplement, chemical or biochemical reagent, or glue.
